(19) United States
(12) Patent Application Publication
(10) Pub. No.: US 2010/0279957 A1
(43) Pub. Date: Nov. 4, 2010

(54) PREDICTING RESPONSIVENESS TO CANCER THERAPEUTICS

(76) Inventors: Anil Potti, Chapel Hill, NC (US); Joseph R. Nevins, Chapel Hill, NC (US); Johnathan M. Lancaster, Tampa, FL (US)

Correspondence Address: MICHAEL BEST & FRIEDRICH LLP, 100 E WISCONSIN AVENUE, Suite 3300, MILWAUKEE, WI, 53202, US

(21) Appl. No.: 12/738,470

(22) PCT Filed: Oct. 20, 2008
(86) PCT No.: PCT/US08/80481

§ 371 Date: Jul. 16, 2010

Related U.S. Application Data
(63) Continuation of application No. 11/975,722, filed on Oct. 19, 2007.

(51) Int. Cl. A61K 31/70; C12Q 1/68; C40B 30/04; C40B 40/08; A61K 31/66; A61K 31/513; A61K 31/437; A61K 31/337; A61K 31/351; G06N 5/02

(52) U.S. Cl. 514/25; 435/6; 506/9; 506/17; 514/110; 514/274; 514/283; 514/449; 514/459; 706/52

(57) ABSTRACT

Provided herein are methods for predicting the responsiveness of a cancer to a chemotherapeutic agent using gene expression profiles. In particular, methods for predicting the responsiveness to 5-fluorouracil, adriamycin, cytotoxan, docetaxol, etoposide, taxol, topotecan, PB kinase inhibitors and Src inhibitors are provided. Methods for developing treatment plans for individuals with cancer are also provided. Kits including gene chips and instructions for predicting responsiveness and computer readable media comprising responsivity information are also provided.

### CROSS-REFERENCE TO RELATED APPLICATIONS

This application claims priority to U.S. Utility application Ser. No. 11/975,722, filed Oct. 19, 2007, which is incorporated herein by reference in its entirety.

### STATEMENT REGARDING FEDERALLY SPONSORED RESEARCH OR DEVELOPMENT

This invention was made with government support under NCI-U54 CA112952-02 and ROI-CA106520 awarded by the National Cancer Institute. The government has certain rights in the invention.

### BACKGROUND OF THE INVENTION

The National Cancer Institute has estimated that in the United States alone, one in three people will be afflicted with cancer. Moreover, approximately 50% to 60% of people with cancer will eventually die from the disease. The inability to predict responses to specific therapies is a major impediment to improving outcome for cancer patients. Because treatment of cancer typically is approached empirically, many patients with chemo-resistant disease receive multiple cycles of often toxic therapy before the lack of efficacy becomes evident. As a consequence, many patients experience significant toxicities, compromised bone marrow reserves, and reduced quality of life while receiving chemotherapy. Further, initiation of efficacious therapy is delayed.

### BRIEF SUMMARY OF THE INVENTION

In one aspect, methods for predicting responsiveness of a cancer to a chemotherapeutic agent are provided. The method includes using a comparison of a first gene expression profile of the cancer to a chemotherapy responsivity predictor set of gene expression profiles to predict the responsiveness of the cancer to the chemotherapeutic agent. The first gene expression profile and the chemotherapy responsivity predictor set each comprise at least five genes from one of Tables 1-8. Tables 1-8 comprise the chemotherapy responsivity predictor set for 5-fluorouracil, adriamycin, cytotoxan, docetaxol, etoposide, taxol, topotecan and PI3 kinase inhibitors, respectively. Also included are methods of predicting the responsiveness to PI3kinase pathway inhibitors and Src pathway inhibitors using the chemotherapy response predictor sets for docetaxol and topotecan, respectively.

In another aspect, methods of developing a treatment plan for an individual with cancer are provided. The predicted responsivity of a cancer to a chemotherapeutic agent may be used to develop a treatment plan for the individual with the cancer. The treatment plan may include administering an effective amount of a chemotherapeutic agent to the individual with the cancer which is predicted to respond to the chemotherapeutic agent.

In yet another aspect, kits including a gene chip for predicting responsivity of a cancer to a chemotherapeutic agent comprising nucleic acids capable of detecting at least five genes selected from any one of Tables 1-8 and instructions for predicting responsivity of a cancer to the chemotherapeutic agents are provided.

In a further aspect, computer readable mediums including gene expression profiles and corresponding responsivity information for chemotherapeutic agents comprising at least five genes from any of Tables 1-8 are provided.

Throughout this specification, reference numbering is sometimes used to refer to the full citation for the references, which can be found in the “Reference Bibliography” after the Examples section. The disclosure of all patents, patent applications, and publications cited herein are hereby incorporated by reference in their entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show a gene expression signature that predicts sensitivity to docetaxel. (A) Strategy for generation of the chemotherapeutic response predictor. (B) Top panel—Cell lines from the NCI-60 panel used to develop the in vitro signature of docetaxel sensitivity. The figure shows a statistically significant difference (Mann Whitney U test of significance) in the IC₅₀/GI₅₀ and LC₅₀ of the cell lines chosen to represent the sensitive and resistant subsets. Bottom Panel—Expression plots for genes selected for discriminating the docetaxel resistant and sensitive NCI-60 cell lines, depicted by color coding with blue representing the lowest level and red the highest. Each column in the figure represents individual samples. Each row represents an individual gene, ordered from top to bottom according to regression coefficients. (C) Top Panel—Validation of the docetaxel response prediction model in an independent set of lung and ovarian cancer cell line samples. A collection of lung and ovarian cell lines were used in a cell proliferation assay to determine the 50% inhibitory concentration (IC₅₀) of docetaxel in the individual cell lines. A linear regression analysis demonstrates a statistically significant (p<0.01, log rank) relationship between the IC₅₀ of docetaxel and the predicted probability of sensitivity to docetaxel. Bottom panel—Validation of the docetaxel response prediction model in another independent set of 29 lung cancer cell line samples (Gemma A, Geo accession number: GSE 4127). A linear regression analysis demonstrates a very significant (p<0.001, log rank) relationship between the IC₅₀ of docetaxel and the predicted probability of sensitivity to docetaxel. (D) Left Panel—A strategy for assessment of the docetaxel response predictor as a function of clinical response in the breast neoadjuvant setting. Middle panel—Predicted probability of docetaxel sensitivity in a collection of samples from a breast cancer single agent neoadjuvant study. Twenty of twenty four samples (91.6%) were predicted accurately using the cell line based predictor of response to docetaxel. Right panel—A single variable scatter plot demonstrating a significance test of the predicted probabilities of sensitivity to docetaxel in the sensitive and resistant tumors (p<0.001, Mann Whitney U test of significance). (E) Left Panel—A strategy for assessment of the docetaxel response predictor as a function of clinical response in advanced ovarian cancer. Middle panel—Predicted probability of docetaxel sensitivity in a collection of samples from a prospective single agent salvage therapy study. Twelve of fourteen samples (85.7%) were predicted accurately using the cell line based predictor of response to docetaxel. Right panel—A single variable scatter plot demonstrating statistical significance (p<0.01, Mann Whitney U test of significance).

FIGS. 2A-2C show the development of a panel of gene expression signatures that predict sensitivity to chemotherapeutic drugs. (A) Gene expression patterns selected for predicting response to the indicated drugs. The genes involved the individual predictors are shown in Tables 1-8, as indicated. (B) Independent validation of the chemotherapy response predictors in an independent set of cancer cell lines³⁷ that have dose response and Affymetrix expression data.³⁸ A single variable scatter plot demonstrating a significance test of the predicted probabilities of sensitivity to any given drug in the sensitive and resistant cell lines (p value, Mann Whitney U test of significance). Red symbols indicate resistant cell lines, and blue symbols indicate those that are sensitive. (C) Prediction of single agent therapy response in patient samples using in vitro cell line based expression signatures of chemosensitivity. In each case, red represents non-responders (resistance) and blue represents responders (sensitivity). The top panel shows the predicted probability of sensitivity to topotecan when compared to actual clinical response data (n=48), the middle panel demonstrates the accuracy of the adriamycin predictor in a cohort of 122 samples (Evans W, GSE650 and GSE651). The bottom panel shows the predictive accuracy of the cell line based paclitaxel (taxol) predictor when used as a salvage chemotherapy in advanced ovarian cancer (n=35). The positive and negative predictive values for all the predictors are summarized in Table 16.

FIGS. 3A-3B show the prediction of response to combination therapy. (A) Top Panel—Strategy for assessment of chemotherapy response predictors in combination therapy as a function of pathologic response. Middle panel—Prediction of patient response to neoadjuvant chemotherapy involving paclitaxel, 5-fluorouracil (5-FU), adriamycin, and cyclophosphamide (TFAC) using the single agent in vitro chemosensitivity signatures developed for each of these drugs. Bottom Panel—Prediction of response (38 non-responders, 13 responders) employing a combined probability predictor assessing the probability of all four chemosensitivity signatures in 51 patients treated with TFAC chemotherapy shows statistical significance (p<0.0001, Mann Whitney) between responders (blue) and non-responders (red). Response was defined as a complete pathologic response after completion of TFAC neoadjuvant therapy. (B) Top Panel—Prediction of patient response (n=45) to adjuvant chemotherapy involving 5-FU, adriamycin, and cyclophosphamide (FAC) using the single agent in vitro chemosensitivity predictors developed for these drugs. Middle panel—Prediction of response (34 responders, 11 non-responders) employing a combined probability predictor assessing the probability of all four chemosensitivity signatures in 45 patients treated with FAC chemotherapy. Bottom panel—Kaplan Meier survival analysis for patients predicted to be sensitive (blue curve) or resistant (red curve) to FAC adjuvant chemotherapy.

FIG. 4 shows patterns of predicted sensitivity to common chemotherapeutic drugs in human cancers. Hierarchical clustering of a collection of breast (n=171), lung cancer (n=91) and ovarian cancer (n=119) samples according to patterns of predicted sensitivity to the various chemotherapeutics. These predictions were then plotted as a heatmap in which high probability of sensitivity/response is indicated by red, and low probability or resistance is indicated by blue.

FIGS. 5A-5B show the relationship between predicted chemotherapeutic sensitivity and oncogenic pathway deregulation. (A) Top Panel—Probability of oncogenic pathway deregulation as a function of predicted docetaxel sensitivity in a series of lung cancer cell lines (red=sensitive, blue=resistant). Bottom panel—Probability of oncogenic pathway deregulation as a function of predicted topotecan sensitivity in a series of ovarian cancer cell lines (red=sensitive, blue=resistant). (B) Top Left Panel—The lung cancer cell lines showing an increased probability of PI3 kinase were also more likely to respond to a PI3 kinase inhibitor (L Y −294002) (p=0.001, log-rank test)), as measured by sensitivity to the drug in assays of cell proliferation. Top Right Panel—Those cell lines predicted to be resistant to docetaxel were more likely to be sensitive to PI3 kinase inhibition (p<0.001, log-rank test). Bottom Left Panel—Ovarian cancer cell lines showing an increased probability of Src pathway deregulation were also more likely to respond to a Src inhibitor (SU6656) (p<0.007, log-rank test). Bottom Right Panel—The relationship between Src pathway deregulation and topotecan resistance can be demonstrated in a set of 13 ovarian cancer cell lines. Ovarian cell lines that are predicted to be topotecan resistant have a higher likelihood of Src pathway deregulation and there is a significant linear relationship (p=0.001, log rank) between the probability of topotecan resistance and sensitivity to a drug that inhibits the Src pathway (SU6656).

FIG. 6 shows a scheme for utilization of chemotherapeutic and oncogenic pathway predictors for identification of individualized therapeutic options.

FIGS. 7A-7C show a patient-derived docetaxel gene expression signature predicts response to docetaxel in cancer cell lines. (A) Top panel—A ROC curve analysis to show the approach used to define a cut-off, using docetaxel as an example. Middle panel—A t-test plot of significance between the probability of docetaxel sensitivity and IC 50 for docetaxel sensitive in cell lines, shown by histologic type. Bottom panel—A linear regression analysis showing the significant correlation between predicted sensitivity and actual sensitivity (IC₅₀) for docetaxel, in lung and ovarian cancer cell lines. (B) Generation of a docetaxel response predictor based on patient data that was then validated in a leave one out cross validation and linear regression analysis (p-value obtained by log-rank), evaluated against the IC₅₀ for docetaxel in two NCI-60 cell line drug screening experiments. (C) A comparison of predictive accuracies between a predictor for docetaxel generated from the cell line data (top panel, accuracy: 85.7%) and a predictor generated from patients treatment data (bottom panel, accuracy: 64.3%) shows the relative inferiority of the latter approach, when applied to an independent dataset of ovarian cancer patients treated with single agent docetaxel.

FIGS. 8A-8C show the development of gene expression signatures that predict sensitivity to a panel of commonly used chemotherapeutic drugs. Panel A shows the gene expression models selected for predicting response to the indicated drugs, with resistant lines on the left, sensitive on the right for each predictor. Panel B shows the leave one out cross validation accuracy of the individual predictors. Panel C demonstrates the results of an independent validation of the chemotherapy response predictors in an independent set of cancer cell lines³⁷ shown as a plot with error bars (blue—sensitive, red—resistant).

FIG. 9 shows the specificity of chemotherapy response predictors. In each case, individual predictors of response to the various cytotoxic drugs was plotted against cell lines known to be sensitive or resistant to a given chemotherapeutic agent (e.g., adriamycin, paclitaxel).

FIG. 10A-10C shows the relationships in predicted probability of response to chemotherapies in breast (A), lung (B) and ovarian (C) cancers. In each case, a regression analysis (log rank) of predicted probability of response to two drugs is shown.

FIG. 11 shows the absolute probabilities of response to various chemotherapies in human lung and breast cancer samples.

FIG. 12 shows a gene expression based signature of PI3 kinase pathway deregulation. Image intensity display of expression levels for genes that most differentiate control cells expressing GFP from cells expressing the oncogenic activity of P13 kinase. The expression value of genes composing each signature is indicated by color, with blue representing the lowest value and red representing the highest level. The panel below shows the results of a leave one out cross validation showing a reliable differentiation between GFP controls (blue) and cells expressing P13 kinase (red).

FIGS. 13A-13C show the relationship between oncogenic pathway deregulation and chemosensitivity patterns (using docetaxel as an example). (A) Probability of oncogenic pathway deregulation as a function of predicted docetaxel sensitivity in the NCI-60 cell line panel (red=sensitive, blue=resistant). (B) Linear regression analysis (log-rank test of significance) to identify relationships between predicted docetaxel sensitivity or resistance and deregulation of PI3 kinase and E2F3 pathways. (C) A non-parametric t-test of significance demonstrating a significant difference in docetaxel sensitivity, between those cell lines predicted to be either pathway deregulated (>50% probability, red) or quiescent (<50% probability, blue), shown for both E2F and PI3 kinase pathways.

FIG. 14 shows a scatter plot demonstrating a linear regression analysis that identifies a statistically significant correlation between probability of docetaxel resistance and PI3 Kinase pathway activation in an independent cohort of 17 non-small cell lung cancer cell lines.

FIG. 15 shows a functional block diagram of general purpose computer system 1500 for performing the functions of the software provided by the invention.

### BRIEF DESCRIPTION OF THE TABLES

Tables 1-8 include the chemotherapy responsivity predictor set for 5-fluorouracil, adriamycin, cytotoxan, docetaxol, etoposide, taxol, topotecan and PI3 kinase inhibitors, respectively.

Tables 9-15 list cell lines and indicate their sensitivity or resistance to 5-fluorouracil, adriamycin, cytotoxan, docetaxol, etoposide, taxol, and topotecan, respectively.

Table 16 is a summary of the chemotherapy response predictors—validations in cell line and patient data sets.

Table 17 shows an enrichment analysis shows that a genomic-guided response prediction increases the probability of a clinical response in the different data sets studied. Table 18 shows the accuracy of genomic-based chemotherapy response predictors as compared to previously reported predictors of response.

### DETAILED DESCRIPTION OF THE INVENTION

The difficulty with administering one or more chemotherapeutic agents to an individual with cancer is that not all individuals with cancer will respond favorably to the chemotherapeutic agent selected by the physician. Frequently, the administration of one or more chemotherapeutic agent results in the individual becoming even more ill from the toxicity of the agent, while the cancer persists. Due to the cytotoxic nature of chemotherapeutic agents, the individual is physically weakened and immunologically compromised such that the individual cannot tolerate multiple rounds of therapy. Hence a personalized treatment plan is highly desirable.

As described in the Examples, the inventors identified gene expression patterns within primary tumors or cell lines that predict response to various chemotherapeutic agents. These predictions may be used to develop treatment plans for individual cancer patients. The invention also provides integrating gene expression profiles that predict responsiveness to combination therapies as a strategy for developing personalized treatment plans for individual patients. Treatment plans may result in individuals having a complete response, a partial response or an incomplete response to the cancer.

A “complete response” (CR) to treatment of cancer is defined as a complete disappearance of all measurable and assessable disease. In ovarian cancer a complete response includes, in the absence of measurable lesions, a normalization of the CA-125 level following adjuvant therapy. An individual who exhibits a complete response is known as a “complete responder.”

An “incomplete response” (IR) includes those who exhibited a “partial response” (PR), had “stable disease” (SD), or demonstrated “progressive disease” (PD) during primary therapy.

A “partial response” refers to a response that displays 50% or greater reduction in bi-dimensional size (area) of the lesion for at least 4 weeks or, in ovarian cancer, a drop in the CA-125 level by at least 50% for at least 4 weeks.

“Progressive disease” refers to response that is a 50% or greater increase in the product from any lesion documented within 8 weeks of initiation of therapy, the appearance of any new lesion within 8 weeks of initiation of therapy, or in the case of ovarian cancer, any increase in the CA-125 from baseline at initiation of therapy.

“Stable disease” was defined as disease not meeting any of the above criteria.

“Effective amount” refers to an amount of a chemotherapeutic agent that is sufficient to exert a prophylactic or therapeutic effect in the subject, i.e., that amount which will stop or reduce the growth of the cancer or cause the cancer to become smaller in size compared to the cancer before treatment or compared to a suitable control. In most cases, an effective amount will be known or available to those skilled in the art. The result of administering an effective amount of a chemotherapeutic agent may lead to effective treatment of the patient. It is desirable for an effective amount to be an amount sufficient to exert cytotoxic effects on cancerous cells.

“Predicting” and “prediction” as used herein includes, but is not limited to, generating a statistically based indication of whether a particular chemotherapeutic agent will be effective to treat the cancer. This does not mean that the event will happen with 100% certainty.

As used herein, “individual” and “subject” are interchangeable. A “patient” refers to an “individual” who is under the care of a treating physician.

The present invention may be practiced using any suitable technique, including techniques known to those skilled in the art. Such techniques are available in the literature or in scientific treatises, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook); Current Protocols in Molecular Biology (F. M. Ausubel et al., eds., 1987, including supplements); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons; Inc., New York, 2000) and Casarett and Doull's Toxicology The Basic Science of Poisons, C. Klaassen, ed., 6th edition (2001).

### Methods for Predicting Responsiveness to Chemotherapy

Methods of predicting responsiveness of a cancer to a chemotherapeutic agent are provided herein. Specifically, the methods rely on using a comparison of a gene expression profile of the cancer to a chemotherapy responsivity predictor set to predict the responsiveness to the chemotherapeutic agent. See Tables 1-8 for the chemotherapeutic responsivity predictor sets. The chemotherapy responsivity predictor set is expected to be distinct for each class of chemotherapeutic agents and may vary between chemotherapeutic agents within the same class. A class of chemotherapeutic agents is chemotherapeutic agents that are similar in some way. For example, the agents may be known to act through a similar mechanism, or have similar targets or structures. An example of a class of chemotherapeutic agents is agents that inhibit PI3 kinase.

The chemotherapy predictor set is, or may be derived from, a set of gene expression profiles obtained from samples (cell lines, tumor samples, etc.) with known sensitivity or resistance to the chemotherapeutic agent. The comparison of the expression of a specific set of genes in the cancer to the same set of genes in samples known to be sensitive or resistant to the chemotherapeutic agent allows prediction of the responsiveness of the cancer to the chemotherapeutic agent. The prediction may indicate that the cancer will respond completely to the chemotherapeutic agent, or it may predict that the cancer will be only partially responsive or non-responsive (i.e. resistant) to the chemotherapeutic agent. The cell lines used to generate the chemotherapy responsivity predictor sets and an indication of the cell lines' sensitivity or resistance to the chemotherapeutic agents are provided in Tables 9-15.

The methods described herein provide an indication of whether the cancer in the patient is likely to be responsive to a particular chemotherapeutic prior to beginning treatment that is more accurate than predictions using population-based approaches from clinical studies. The methods allow identification of chemotherapeutics estimated to be useful in combating a particular cancer in an individual patient, resulting in a more cost-effective, targeted therapy for the cancer patient and avoiding side effects from non-efficacious chemotherapeutic agents.

Tables 1-8 also provide the relative “weights” of each of the individual genes that make up the responsivity predictor set. The weights demonstrate that some genes are more strongly indicative of sensitivity or resistance of a cancer to a particular therapeutic agent. Predictions based on the complete set of genes are expected to provide the most accurate predictions regarding the efficacy of treating the cancer with a particular therapeutic agent. Those of skill in the art will understand based on the weights of each gene in the responsivity predictor set that some genes are more predictive of outcome than others and thus that the entire responsivity predictor set need not be used to develop a useful prediction.

Once an individual's cancer is predicted to be responsive to a particular chemotherapy, then a treatment plan can be developed incorporating the chemotherapeutic agent and an effective amount of the chemotherapeutic agent(s) may be administered to the individual with the cancer. Those of skill in the art will appreciate that the methods do not guarantee that the individuals will be responsive to the chemotherapeutic agent, but the methods will increase the probability that the selected treatment will be effective to treat the cancer. Also encompassed is the ability to predict the responsiveness of the cancer to multiple chemotherapeutic agents and then to develop a treatment plan using a combination of two or more chemotherapeutic agents. Those of skill in the art appreciate that combination therapy is often suitable.

Treatment or treating a cancer includes, but is not limited to, reduction in cancer growth or tumor burden, enhancement of an anti-cancer immune response, induction of apoptosis of cancer cells, inhibition of angiogenesis, enhancement of cancer cell apoptosis, and inhibition of metastases. Administration of an effective amount of a chemotherapeutic agent to a subject may be carried out by any means known in the art including, but not limited to intraperitoneal, intravenous, intramuscular, subcutaneous, transcutaneous, oral, nasopharyngeal or transmucosal absorption. The specific amount or dosage administered in any given case will be adjusted in accordance with the specific cancer being treated, the condition, including the age and weight, of the subject, and other relevant medical factors known to those of skill in the art.

In one embodiment, the methods involve predicting responsiveness to chemotherapeutic agents of an individual with cancer. Cancers include but are not limited to any cancer treatable with the chemotherapeutic agents described herein. Cancers include, but are not limited to, ovarian cancer, lung cancer, prostrate cancer, renal cancer, colon cancer, leukemia, skin cancer, brain or central nervous system cancer and breast cancer. In another embodiment, the individual has advanced stage cancer (e.g., Stage III/IV ovarian cancer). In other embodiments, the individual has early stage cancer. For the individuals with advanced cancer, one form of primary treatment practiced by treating physicians is to surgically remove as much of the tumor as possible, a practice sometime known as “debulking.”

The sample of the cancer used to obtain the first gene expression profile may be directly from a tumor that was surgically removed. Alternatively, the sample of the cancer could be from cells obtained in a biopsy or other tumor sample. A sample from ascites surrounding the tumor may also be used.

The sample is then analyzed to obtain a first gene expression profile. This can be achieved by any suitable means, including those available to those of skill in the art. One method that can be used is to isolate RNA (e.g., total RNA) from the cellular sample and use a publicly or commercially available micro array system to analyze the gene expression profile from the cellular sample. One microarray that may be used is Affymetrix Human U133A chip. One of skill in the art follows the standard directions that come with a commercially available microarray. Other types of microarrays may be used, for example, microarrays using RT-PCR for measurement. Other sources of microarrays include, but are not limited to, Stratagene (e.g., Universal Human Microarray), Genomic Health (e.g., Oncotype DX chip), Clontech (e.g., Atlas™ Glass Microarrays), and other types of Affymetrix microarrays. In one embodiment, the microarray may be made by a researcher or obtained from an educational institution. In other embodiments, customized microarrays, which include the particular set of genes that are particularly suitable for prediction, can be used. The gene expression profile may be obtained by any other means, including those known to those of skill in the art, e.g., Northern blots, real time rt-PCR, Western blots for the expressed proteins or protein assays.

Once a first gene expression profile has been obtained from the sample, it is compared with chemotherapy responsivity predictor set of gene expression profiles. Tables 1-8 describe the chemotherapy responsivity predictor sets for 5-FU, adriamycin, cytotoxan, docetaxol, etoposide, taxol, topotecan, and PI3 kinase inhibitors, respectively.

The use of the chemotherapy responsitivity predictor set in its entirety is contemplated; however, it is also possible to use subsets of the predictor set. For example, a subset of at least 2, 5, 10, 15, 20, 25, 30, 35 or 40 or more genes from one of Tables 1-8 can be used for predictive purposes. For example, 40, 45, 50, 55, 60, 65, 70, 75 or 80 genes from Table 7 could be used in a topotecan chemotherapy responsivity predictor set.

Thus, one of skill in art may use the chemotherapy responsitivity predictor set as detailed in the Examples to predict whether an individual or patient with cancer will be responsive to the selected chemotherapeutic agent. If the individual is a complete responder to a chemotherapeutic agent, then a treatment plan may be designed in which the therapeutic agent will be administered in an effective amount. If the complete responder stops being a complete responder, as sometimes happens, then the first gene expression profile may be further analyzed for responsivity to an alternative agent to determine which alternative agent should be administered to most effectively combat the cancer while minimizing the toxic side effects to the individual. If the individual is an incomplete responder, then the individual's gene expression profile can be further analyzed for responsivity to an alternative agent to determine which agent should be administered, or alternatively which combination of agents is predicted to be most effective to treat the cancer.

Those of skill in the art will understand that the first gene expression profile may be tested against more than one chemotherapy responsivity predictor set to allow development of a treatment plan with the best likelihood of treating the individual with the cancer. For example, an individual can be evaluated for responsiveness to one or more chemotherapeutic agents. In certain embodiments, the methods of the application are performed outside of the human body. In addition, an individual can be assessed to determine if they will be refractory to a commonly used first-line therapy such that additional alternative therapeutic intervention can be started.

For the individuals who appear to be incomplete responders to a chemotherapeutic agent or for those individuals who have ceased being complete responders, an important step in the treatment is to determine other alternative cancer therapies that may be administered to the individual to best combat the cancer while minimizing the toxicity of these additional agents.

Alternative therapeutic agents include, but are not limited to, cisplatin, denopterin, edatrexate, methotrexate, nolatrexed, pemetrexed, piritrexim, pteropterin, raltitrexed, trimetrexate, cladribine, clofarabine, fludarabine, 6-mercaptopurine, nelarabine, thiamiprine, thioguanine, tiazofurin, ancitabine, azacitidine, 6-azauridine, capecitabine, carmofur, cytarabine, decitabine, doxifluridine, enocitabine, floxuridine, fluorouracil, gemcitabine, tegafur, troxacitabine, pentostatin, hydroxyurea, cytosine arabinoside, docetaxel, paclitaxel, abraxane, topotecan, adriamycin, etoposide, fluorouracil (5-FU), and cyclophosphamide. In one embodiment, the agent may be selected from platinum-based chemotherapeutic agents (e.g., cisplatin), alkylating agents (e.g., nitrogen mustards), antimetabolites (e.g., pyrimidine analogs), radioactive isotopes (e.g., phosphorous and iodine), miscellaneous agents (e.g., substituted ureas) and natural products (e.g., vinca alkyloids and antibiotics). In another embodiment, the therapeutic agent may be selected from the group consisting of allopurinol sodium, dolasetron mesylate, pamidronate disodium, etidronate, fluconazole, epoetin alfa, levamisole HeL, amifostine, granisetron HCL, leucovorin calcium, sargramostim, dronabinol, mesna, filgrastim, pilocarpine HCl, octreotide acetate, dexrazoxane, ondansetron HCL, ondanselron, busulfan, carboplatin, cisplatin, thiotepa, melphalan HCl, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, pllmycin, mitomycin, pentostatin, mitoxantrone, valrubicin, cytarabine, tludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alpha-2b, mitotane, procarbazine HCL, vinorelbine tartrate, E. coli l-asparaginase, Erwinia L-asparaginase, vincristine sulfate, denileukin diftitox, aldesleukin, rituximab, interferon alpha-1a, paclitaxel, abraxane, docetaxel, BCG live (intravesical), vinblastine sulfate, etoposide, tretinoin, teniposide, porfuner sodium, tluorouracil, betamethasone sodium phosphate and betamethasone acetate, letrozole, etoposide citrororum factor, folinic acid, calcium leucouorin, 5-fluorouricil, adriamycin, c}toxan, and diamino-dichloro-platinum.

In another aspect, the first gene expression profile from the individual with cancer is analyzed and compared to gene expression profiles (or signatures) that are reflective of deregulation of various oncogenic signal transduction pathways. In one embodiment, the alternative cancer therapeutic agent is directed to a target that is implicated in oncogenic signal transduction deregulation. Such targets include, but are not limited to, Src, myc, beta-catenin and E2F3 pathways. Thus, in one aspect, the invention contemplates using an inhibitor that is directed to one of these targets as an additional therapy for cancer. One of skill in the art will be able to determine the dosages for each specific chemotherapeutic agent.

As shown in Example 1, the teachings herein provide a gene expression model that predicts response to docetaxel therapy. The other Examples provide predictors for 5-FU, adriamycin, cytotoxan, taxol, etoposide, topotecan, PI3 kinase inhibitors and Src inhibitors. The gene expression model was developed by using Bayesian binary regression analysis to identify genes highly correlated with drug sensitivity. The developed models were validated in a leave-one-out cross validation.

### Chemotherapy Responsivity Predictor Set of Gene Expression Profiles

The chemotherapy responsitivity predictor sets were created by a method described in detail in the Examples and similar to that detailed in Potti et al. (Genomic signatures to guide the use of chemotherapeutics. Nature Medicine 12(11): 1294-1300, 2006, incorporated herein by reference). Unless otherwise noted in the Examples, the [−log 10(M)] GI50/IC50 and LC50 (50% cytotoxic dose) data on the NCI-60 cell line panel for each of the indicated therapeutic agents was used to populate a matrix with MATLAB software with the relevant expression data for each individual cell line. When multiple entries for a drug screen existed (by NCS number), the entry with the largest number of replicates was included. To develop in vitro gene expression based predictors for chemotherapeutic agent sensitivity from the pharmacologic data used in the NCI-60 drug screen studies, we chose cell lines within the NCI-60 panel that would represent the extremes of sensitivity (See Tables 9-15). Relevant expression data (updated data available on the Affymetrix U95A2 GeneChip) for the selected NCI-60 cell lines were then used in a supervised analysis using Bayesian regression methodologies, as described previously (Pittman J, Huang E, Nevins J, et al: Bayesian analysis of binary prediction tree models for retrospectively sampled outcomes. Biostatistics 5(4):587-601, 2004), to develop a probit model predictive of sensitivity to the indicated chemotherapeutic agent.

Method of Treating Individuals with Cancer

The methods described herein also include treating an individual afflicted with cancer. This method involves administering an effective amount of a chemotherapeutic agent to those individuals predicted to be responsive to such therapy. In the alternative, an effective amount of a combination of chemotherapeutic agents may be administered to individuals predicted to be responsive to combination therapy. In the instance where the individual is predicted to be a non-responder, a physician may decide to administer alternative therapeutic agents alone. In many instances, the treatment will comprise a combination of chemotherapeutic agents.

The methods described herein include, but are not limited to, treating individuals afflicted with NSCLC, breast cancer and ovarian cancer. In one aspect, a chemotherapeutic agent is administered in an effective amount by itself (e.g., for complete responders). In another embodiment, the therapeutic agent is administered with an alternative chemotherapeutic in an effective amount concurrently. In another embodiment, the two therapeutic agents are administered in an effective amount in a sequential manner. In yet another embodiment, the alternative therapeutic agent is administered in an effective amount by itself. In yet another embodiment, the alternative therapeutic agent is administered in an effective amount first and then followed concurrently or step-wise by a second or third chemotherapeutic agent.

Methods of Predicting/Estimating the Efficacy of a Therapeutic Agent in Treating an Individual Afflicted with Cancer

One aspect of the invention provides a method for predicting, estimating, aiding in the prediction of, or aiding in the estimation of, the efficacy of a therapeutic agent in treating a subject afflicted with cancer. In certain embodiments, the methods of the application are performed outside of the human body.

One method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a chemotherapy responsivity predictor set; and (c) averaging the predictions of one or more statistical tree models applied to the values of the metagenes, wherein each model includes one or more nodes, each node representing a metagene, each node including a statistical predictive probability of tumor sensitivity to the therapeutic agent, thereby estimating the efficacy of a therapeutic agent in a subject afflicted with cancer. Another method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a chemotherapy responsivity predictor set; and (c) averaging the predictions of one or more binary regression models applied to the values of the metagenes, wherein each model includes a statistical predictive probability of tumor sensitivity to the therapeutic agent, thereby estimating the efficacy of a therapeutic agent in a subject afflicted with cancer.

In one embodiment, the methods predict the efficacy of a therapeutic agent in treating a subject afflicted with cancer with at least 70% accuracy. In another embodiment, the methods predict the efficacy of a therapeutic agent in treating a subject afflicted with cancer with at least 80% accuracy. In another embodiment, the methods predict the efficacy of a therapeutic agent in treating a subject afflicted with cancer with at least 85% accuracy. In another embodiment, the methods predict the efficacy of a therapeutic agent in treating a subject afflicted with cancer with at least 90% accuracy. In another embodiment, the methods predict the efficacy of a therapeutic agent in treating a subject afflicted with cancer with at least 70%, 75%, 80%, 85%, 90% or 95% accuracy when tested against a validation sample. In another embodiment, the methods predict the efficacy of a therapeutic agent in treating a subject afflicted with cancer with at least 70%, 75%, 80%, 85%, 90% or 95% accuracy when tested against a set of training samples. In another embodiment, the methods predict the efficacy of a therapeutic agent in treating a subject afflicted with cancer with at least 70%, 75%, 80%, 85%, 90% or 95% accuracy when tested on human primary tumors ex vivo or in vivo. Accuracy is the ability of the methods to predict whether a cancer is sensitive or resistant to the chemotherapeutic agent.

The methods predict the efficacy of a therapeutic agent to treat a subject with cancer with at least 70%, 75%, 80%, 85%, 90%, 95%, 97%, 98%, 99% or 100% sensitivity for a particular chemotherapeutic agent. In another embodiment, the methods predict the efficacy of a therapeutic agent in treating a subject afflicted with cancer with at least 70%, 75%, 80%, 85%, 90%, 95%, 97%, 98%, 99% or 100% sensitivity when tested against a validation sample. In another embodiment, the methods predict the efficacy of a therapeutic agent in treating a subject afflicted with cancer with at least 70%, 75%, 80%, 85%, 90%, 95%, 97%, 98%, 99% or 100% sensitivity when tested against a set of training samples. In another embodiment, the methods predict the efficacy of a therapeutic agent in treating a subject afflicted with cancer with at least 70%, 75%, 80%, 85%, 90%, 95%, 97%, 98%, 99% or 100% sensitivity when tested on human primary tumors ex vivo or in vivo. Sensitivity measures the ability of the methods to predict all cancers that will be sensitive to the chemotherapeutic agent.

### (A) Sample of the Cancer

In one embodiment, the methods comprise determining the expression level of genes in a tumor sample from the subject. In certain embodiments, the tumor is a breast tumor, an ovarian tumor, or a lung tumor. In one embodiment, the tumor is not a breast tumor. In one embodiment, the tumor is not an ovarian tumor. In one embodiment, the tumor is not a lung tumor. In one embodiment of the methods described herein, the methods comprise the step of surgically removing a tumor sample from the subject, obtaining a tumor sample from the subject, or providing a tumor sample from the subject.

Alternatively, the sample may be derived from cells from the cancer, or cancerous cells. In another embodiment, the cells may be from ascites surrounding the tumor. The sample may contain nucleic acids from the cancer. Any method may be used to remove the sample from the patient.

In one embodiment, at least 40%, 50%, 60%, 70%, 80% or 90% of the cells in the sample are cancer cells. In preferred embodiments, samples having greater than 50% cancer cell content are used. In one embodiment, the sample is a live tumor sample. In another embodiment, the sample is a frozen sample. In one embodiment, the sample is one that was frozen within less than 5, 4, 3, 2, 1, 0.75, 0.5, 0.25, 0.1, or 0.05 hours after extraction from the patient. Frozen samples include those stored in liquid nitrogen or at a temperature of about −80° C. or below.

### (B) Gene Expression

The expression of the genes may be determined using any method known in the art for assaying gene expression. Gene expression may be determined by measuring mRNA or protein levels for the genes. In one embodiment, an mRNA transcript of a gene may be detected for determining the expression level of the gene. Based on the sequence information provided by the GenBank™ database entries, the genes can be detected and expression levels measured using techniques well known to one of ordinary skill in the art, including but not limited to rtPCR, Northern blot analysis and microarray analysis. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can be also carried out on a DNA array. The use of an array is suitable for detecting the expression level of a plurality of the genes. As another example, the sequences can be used to construct primers for specifically amplifying the polynucleotides in, e.g., amplification-based detection methods such as reverse-transcription based polymerase chain reaction (RT-PCR). As another example, mRNA levels can be assayed by quantitative RT-PCR. Furthermore, the expression level of the genes can be analyzed based on the biological activity or quantity of proteins encoded by the genes. Methods for determining the quantity of the protein include immunoassay methods such as Western blot analysis.

In one exemplary embodiment, about 1-50 mg of cancer tissue was added to a chilled tissue pulverizer, such as to a BioPulverizer H tube (Bio101 Systems, Carlsbad, Calif.). Lysis buffer, such as from the Qiagen RNeasy Mini kit, was added to the tissue and homogenized. A device such as a Mini-Beadbeater (Biospec Products, Bartlesville, Okla.) was used. Tubes were spun briefly as needed to pellet the mixture and reduce foam. The resulting lysate was passed through syringes, such as a 21 gauge needle, to shear DNA. Total RNA was extracted using commercially available kits, such as the Qiagen RNeasy Mini kit. The samples were prepared and arrayed using Affymetrix U133 plus 2.0 GeneChips or Affymetrix U133A GeneChips. Any suitable gene chip may be used.

In one exemplary embodiment, total RNA was extracted using the Qiashredder and Qiagen RNeasy Mini kit and the quality of RNA was checked by an Agilent 2100 Bioanalyzer. The targets for Affymetrix DNA microarray analysis were prepared according to the manufacturer's instructions. Biotin-labeled cRNA, produced by in vitro transcription, was fragmented and hybridized to the Affymetrix U133A GeneChip arrays at 45° C. for 16 hrs and then washed and stained using the GeneChip Fluidics. The arrays were scanned by a GeneArray Scanner and patterns of hybridization were detected as light emitted from the fluorescent reporter groups incorporated into the target and hybridized to oligonucleotide probes. Full details of the methods used for RNA extraction and development of gene expression data from lung and ovarian tumors have been described previously. (Bild A, Yao G, Chang J T, et al: Oncogenic pathways signatures in human cancers as guide to targeted therapies. Nature 439(7074):353-357, 200, Potti A, Dressman H K, Bild A, et al: Genomic signatures to guide the use of chemotherapeutics. Nature Medicine 12(11): 1294-1300, 2006).

In one embodiment, determining the expression level (or obtaining a first gene expression profile) of multiple genes in a tumor sample from the subject comprises extracting a nucleic acid sample from the sample from the subject. In certain embodiments, the nucleic acid sample is an mRNA sample. In one embodiment, the expression level of the nucleic acid is determined by hybridizing the nucleic acid, or amplification products thereof, to a DNA microarray. Amplification products may be generated, for example, with reverse transcription, optionally followed by PCR amplification of the products.

### (C) Genes Screened

In one embodiment, the predictive methods of the invention comprise determining the expression level of all the genes in the cluster that define at least one therapeutic sensitivity/resistance determinative metagene. In one embodiment, the predictive methods of the invention comprise determining the expression level of at least 50%, 60%, 70%, 80%, 90%, 95%, 98%, 99% of the genes in each of the clusters that defines 1 or 2 or more therapeutic sensitivity/resistance determinative metagenes. A metagene is a cluster or set of genes which may be used to predict sensitivity or resistance to a therapeutic agent.

In one embodiment, at least 50%, 60%, 70%, 80%, 90%, 95%, 98%, 99% of the genes whose expression levels are used in order to predict sensitivity to the chemotherapeutic agent (or the genes in the cluster that define a metagene having said predictivity) are genes listed in one of Tables 1-8. In one embodiment, at least 50%, 60%, 70%, 80%, 90%, 95%, 98%, 99% of the genes whose expression levels are determined to predict sensitivity to more than one chemotherapeutic agent (or the genes in the cluster that define a metagene having said predictivity) includes genes listed in more than one of Tables 1-8.

In one embodiment, at least 50%, 60%, 70%, 80%, 90%, 95%, 98%, 99% of the genes listed in one of Tables 1-8 are used to predict responsiveness of a cancer to the corresponding chemotherapeutic agent. Tables 1-8 show the genes in the cluster that are used to define metagenes and indicate the therapeutic agent whose sensitivity it predicts.

In one embodiment, at least 50%, 60%, 70%, 80%, 90%, 95%, 98%, 99% of the genes whose expression levels are determined to predict 5-FU sensitivity (or the genes in the cluster that define a metagene having said predictivity) are genes represented by the following symbols: LOC92755 (TUBB, LOC648765), CDKN2A, TRA@, GABRA3, COL1lA2, ACTB, PDLIM4, ACTA2, FTSJ1, NBR1 (LOC727732), CFL1, ATP1A2, APOC4, KlAA1509, ZNF516, GRIK5, PDE5A, ARSF, ZC3H7B, WBP4, CSTB, TSPY1 (TSPY2, LOC653174, LOC728132, LOC728137, LOC728395, LOC728403, LOC728412), HTR2B, KBTBD11, SLC25A17, HMGN3, FIBP, IFT140, FAM63B, ZNF337, KlAA0100, FAM13C1, STK25, CPNE1, PEX19, EIF5B, EEF1A1 (APOLD1, LOC440595), SRR, THEM2, ID4, GGT1 (GGTL4), IFNα10, TUBB2A (TUBB4, TUBB2B), and TUBB3.

In one embodiment, at least 50%, 60%, 70%, 80%, 90%, 95%, 98%, 99% of the genes whose expression levels are determined to predict adriamycin sensitivity are genes represented by the following symbols: MLANA, CSPG4, DDR2, ETS2, EGFR, BIK, CD24, ZNF185, DSCR1, GSN, TPST1, LCN2, FAIM3, NCK2, PDZRN3, FKBP2, KRT8, NRP2, PKP2, CLDN3, CAPN1, STXBP1, LY96, WWC1, C10orf56, SPINT2, MAGED2, SYNGR2, SGCD, LAMC2, C19orf21, ZFHX1B, KRT18, CYBA, DSP, ID1, ID1, PSAP, ZNF629, ARHGAP29, ARHGAP8 (LOC553158), GPM6B, EGFR, CALU, KCNK1, RNF144, FEZ1, MEST, KLF5, CSPG4, FLNB, GYPC, SLC23A2, MITF, PITPNM1, GPNMB, PMP22, PLXNB3 (SRPK3), MIA, RAB40C, MAD2L1BP, PLOD3, VIL2, KLF9, PODXL, ATP6V1B2, SLC6A8, PLP1, KRT7, PKP3, DLG3, ZHX2, LAMAS, SASH1, GAS1, TACSTD1, GAS1, and CYP27A1.

In one embodiment, at least 50%, 60%, 70%, 80%, 90%, 95%, 98%, 99% of the genes whose expression levels are determined to predict cytoxan sensitivity (or the genes in the cluster that define a metagene having said predictivity) are genes represented by the following symbols: DAP3, RPS9, TTR, ACTB, MARCKS, GGT1 (GGT2), GGTL4, GGTLA4, LOC643171, LOC653590, LOC728226, LOC728441, LOC729S38, LOC73 1629), FANCA, CDC42EP3, TSPAN4, C60rf145, ARNT2, KIF22 (LOC728037), NBEAL2, CA V1, SCRN1, SCHIP1, PHLDB1, AKAP12, ST5, SNAI2, ESD, ANP32B, CD59, ACTN1, CD59, PEG10, SMARCA1, GGCX, SAMD4A, CNN3, LPP, SNRPF, SGCE, CALD1, and C220rf5.

In one embodiment, at least 50%, 60%, 70%, 80%, 90%, 95%, 98%, 99% of the genes whose expression levels are determined to predict docetaxel sensitivity (or the genes in the cluster that define a metagene having said predictivity) are genes represented by the following symbols: BLR1, EIF4A2, FLT1, BAD, PIP5K3, BIN1, YBX1, BCKDK, DOHH, FOXD1, TEX261, NBR1 (LOC727732), APOA4, DDX5, TBCA, USP52, SLC25A36, CHP, ANKRD28, PDXK, ATP6AP1, SETD2, CCS, BRD2, ASPHD1, B4GALT6, ASL, CAPZA2, STARD3, LIMK2 (PPPIR14BP1), BANF1, GNB2, ENSA, SH3GL1, ACVR1B, SLC6A1, PPP2R1A, PCGF1, LOC643641, INPP5A, TLE1, PLLP, ZKSCAN1, TIAL1, TK1, PPP2R1A, and PSMB6.

In one embodiment, at least 50%, 60%, 70%), 80%, 90%, 95%, 98%, 99% of the genes whose expression levels are determined to predict etoposide sensitivity are genes represented by the following symbols: LIMK1, LIG3, AXL, IFI16, MMP14, GRB7, VAV2, FLT1, JUP, FN1, FN1, PKM2, LYPLA3, RFTN1, LAD1, SPINT1, CLDN3, PTRF, SPINT2, MMP14, FAAH, CLDN4, ST14, C19orf21, KIAA0506, LLGL2 (MADD), COBL, ZFHX1B, GBP1, lER2, PPL, TMEM30B, CNKSR1, CLDN7, BTN3A2, BTN3A2, TUBB2A, MAP7, HNRNPG-T, UGCG, GAK, PKP3, DFNA5, DAB2, TACSTD1, SPARC, and PPP2R5A.

In one embodiment, at least 50%, 60%, 70%, 80%, 90%, 95%, 98%, 99% of the genes whose expression levels are determined to predict taxol sensitivity (or the genes in the cluster that define a metagene having said predictivity) are genes represented by the following symbols: NR2F6, TOP2B, RARG, PCNA, PTPN11, ATM, NFATC4, CACNG1, C22orf31, PIK3R2, PRSS12, MYH8, SCCPDH, PHTF2, IQSEC2, TRPC3, TRAFD1, HEPH, SOX30, GATM, LMNA, HD, YIPF3, DNPEP, PCDH9, KLHDC3, SLC10A3, LHX2, CKS2, SECTM1, SF1, RPS6KA4, DYRK2, GDI2, and IFI30.

In one embodiment, at least 50%, 60%, 70%, 80%, 90%, 95%, 98%, 99% of the genes whose expression levels are determined to predict topotecan sensitivity (or the genes in the cluster that define a metagene having said predictivity) are genes represented by the following symbols: DUSP1, THBS1, AXL, RAP1GAP, QSCN6, IL1R1, TGFBI, PTX3, BLM, TNFRSF1A, FGF2, VEGFC, ACO2, FARSLA, RIN2, FGF2, RRAS, FIGF, MYB, CDH2, FGFR1, FGFR1, LAMC1, HIST1H4K (HIST1H4J), COL6A2, TMC6, PEA15, MARCKS, CKAP4, GJA1, FBN1, BASP1, BASP1, BTN2A1, ITGB1, DKFZP686A01247, MYLK, LOXL2, HEG1, DEGS1, CAP2, CAP2, PTGER4, BAI2, NUAK1, DLEU1 (SPANXC), RAB11FIP5, FSTL3, MYL6, VIM, GNAl2, PRAF2, PTRF, CCL2, PLOD2, COL6A2, ATP5G3, GSR, NDUFS3, ST14, NID1, MYO1D, SDHB, CAV1, DPYSL3, PTRF, FBXL2, RIN2, PLEKHC1, CTGF, COL4A2, TPM1, TPM1, TPM1, FZD2, LOXL1, SYK, HADHA, TNFAIP1, NNMT, HPGD, MRC2, MEIS3P1, AOX1, SEMA3C, SEMA3C, SYNE1, SERPINE1, IL6, RRAS, GPD1L, AXL, WDR23, CLDN7, IL15, TNFAIP2, CYR61, LRP1, AMOTL2, PDE1B, SPOCK1, RAI14, PXDN, COL4A1, C1R, KIAA0802 (C21orf57), C50rf13, TUFM, EDIL3, BDNF, PRSS23, ATP5A1, FRAT2, C16orf51, TUSC4, NUP50, TUBA3, NFIB, TLE4, AKT3, CRIM1, RAD23A, COX5A, SMCR7L, MXRA7, STARD7, STC1, TTC28, PLK2, TGDS, CALD1, OPTN, IFITM3, DFNA5, FGFR1, HTATIP, SYK, LAMB1, FZD2, SERPINE1, THBS1, CCL2, ITGA3, ITGA3, and UBE2A.

### (D) Metagene Valuation

In one embodiment, the predictive methods of the invention comprise defining the value of one or more metagenes from the expression levels of the genes. A metagene value is defined by extracting a single dominant value from a cluster of genes associated with sensitivity to an anti-cancer agent.

In one embodiment, the dominant single value is obtained using single value decomposition (SVD). In one embodiment, the cluster of genes of each metagene or at least of one metagene comprises at least 3, 4, 5, 6, 7, 8, 9, 10, 12, 15, 18, 20 or 25 genes.

In one embodiment, the predictive methods of the invention comprise defining the value of at least one metagene wherein the genes in the cluster of genes from which the metagene is defined, shares at least 50%, 60%, 70%, 80%, 90%, 95% or 98% of genes in common to the genes in one of Tables 1-8. In one embodiment, the predictive methods of the invention comprise defining the value of at least two metagenes, wherein the genes in the cluster of genes from which each metagene is defined share at least 50%, 60%, 70%, 80%, 90%, 95% or 98% of genes in common to the genes in any one of Tables 1-8. In one embodiment, the predictive methods of the invention comprise defining the value of a metagene from a cluster of genes, wherein at least 2, 3, 4, 5, 6, 7, 8, 9, 10, 11, 12, 13, 14, 15, 16, 17, 18, 19 or 20 genes in the cluster are selected from the genes listed in one of Tables 1-8.

In one embodiment, the clusters of genes that define each metagene were identified using supervised classification methods of analysis as previously described. See, for example, West, M. et al. Proc Natl Acad Sci USA 98, 11462-11467 (2001). A set of genes whose expression levels are most highly correlated with the classification of tumor samples into sensitivity to an anti-cancer agent versus no sensitivity to an anti-cancer agent were selected. The dominant principal components from such a set of genes defines a relevant phenotype-related metagene, and regression models, such as binary regression models, were used to assign the relative probability of sensitivity to an anti-cancer agent.

(E) Predictions from Tree Models

In one embodiment, the methods comprise averaging the predictions of one or more statistical tree models applied to the metagene values, wherein each model includes one or more nodes, each node representing a metagene, each node including a statistical predictive probability of sensitivity to an anti-cancer agent. The statistical tree models may be generated using the methods described herein for the generation of tree models. General methods of generating tree models may also be found in the art (See for example Pitman et al, Biostatistics 2004; 5:587-601; Denison et al. Biometrika 1999; 85:363-77; Nevins et al. Hum Mol Genet 2003; 12:R153-7; Huang et al. Lancet 2003; 361: 1590-6; West et al. Proc Natl A cad Sci USA 2001; 98:11462-7; U.S. Patent Pub. Nos. 2003-0224383; 2004-0083084; 2005-0170528; 2004-0106113; and U.S. application Ser. No. 11/198,782).

In one embodiment, the methods comprise deriving a prediction from a single statistical tree model, wherein the model includes one or more nodes, each node representing a metagene, each node including a statistical predictive probability of sensitivity to an anti-cancer agent. In alternative embodiments, the tree may comprise at least 2, 3, 4, or 5 nodes.

In one embodiment, the methods comprise averaging the predictions of one or more statistical tree models applied to the metagene values, wherein each model includes one or more nodes, each node representing a metagene, each node including a statistical predictive probability of sensitivity to an anti-cancer agent. Accordingly, the invention provides methods that use mixed trees, where a tree may contain at least two nodes, where each node represents a metagene representative of the sensitivity/resistance to a particular agent.

In one embodiment, the statistical predictive probability was derived from a Bayesian analysis. In another embodiment, the Bayesian analysis included a sequence of Bayes factor based tests of association to rank and select predictors that define a node binary split, the binary split including a predictor/threshold pair. Bayesian analysis is an approach to statistical analysis that is based on the Bayes law, which states that the posterior probability of a parameter p is proportional to the prior probability of parameter p multiplied by the likelihood of p derived from the data collected. This methodology represents an alternative to the traditional (or frequentist probability) approach: whereas the latter attempts to establish confidence intervals around parameters, and/or falsify a-priori null-hypotheses, the Bayesian approach attempts to keep track of how a priori expectations about some phenomenon of interest can be refined, and how observed data can be integrated with such a priori beliefs, to arrive at updated posterior expectations about the phenomenon. Bayesian analysis has been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” nodes using a splitting rule. For instance, for clarification, training data contains sample vectors that have one or more measurement variables and one variable that determines that class of the sample. Various splitting rules may be used. A statistical predictive tree model to which Bayesian analysis is applied may consistently deliver accurate results with high predictive capabilities. Other statistical models known to those of skill in the art may be used.

Gene expression signatures that reflect the activity of a given pathway may be identified using supervised classification method of analysis previously described (e.g., West, M. et al. Proc Natl Acad Sci USA 98, 11462-11467, 2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models assign the relative probability of sensitivity to an anti-cancer agent.

In one embodiment, each statistical tree model generated by the methods described herein comprises 2, 3, 4, 5, 6 or more nodes. In one embodiment of the methods described herein for defining a statistical tree model predictive of sensitivity/resistance to a therapeutic, the resulting model predicts cancer sensitivity to an anti-cancer agent with at least 70%, 80%, 85%, or 90% or higher accuracy. In another embodiment, the model predicts sensitivity to an anti-cancer agent with greater accuracy than clinical variables. In one embodiment, the clinical variables are selected from age of the subject, gender of the subject, tumor size of the sample, stage of cancer disease, histological subtype of the sample and smoking history of the subject. In one embodiment, the cluster of genes that define each metagene comprise at least 3, 4, 5, 6, 7, 8, 9, 10, 12 or 15 genes. In one embodiment, the correlation-based clustering is Markov chain correlation-based clustering or K-means clustering.

### Gene Chips and Kits

Arrays and microarrays which contain the gene expression profiles for determining responsivity to the chemotherapeutic agents as disclosed here are also encompassed within the scope of this invention. Methods of making arrays are well-known in the art and as such do not need to be described in detail here.

Such arrays can contain the profiles of 5, 10, 15, 20, 25, 30, 40, 50, 75, 100, 150, 200 or more genes as disclosed in the Tables. Accordingly, arrays for detection of responsivity to particular therapeutic agents can be customized for diagnosis or treatment of specific cancers, such as ovarian cancer, breast cancer, or NSCLC. The array can be packaged as part of kit comprising the customized array itself and a set of instructions for how to use the array to determine an individual's responsivity to a specific cancer therapeutic agent.

Also provided are reagents and kits for practicing one or more of the above described methods. The subject reagents and kits thereof may vary greatly. Reagents of interest include reagents specifically designed for use in production of the above described metagene values.

One type of such reagent is an array probe of nucleic acids, such as a DNA chip, in which the genes defining the metagenes in the therapeutic efficacy predictive tree models are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; as well as WO 95/21265; WO 96/31622; WO 97/10365; WO 97/27317; EP 373 203; and EP 785 280; the disclosures of which are herein incorporated by reference.

The DNA chip is conveniently used to compare the expression levels of a number of genes at the same time. DNA chip-based expression profiling can be carried out, for example, by the method as disclosed in “Microarray Biochip Technology” (Mark Schena, Eaton Publishing, 2000). A DNA chip comprises immobilized high-density probes to detect a number of genes. Thus, the expression levels of many genes can be estimated at the same time by a single-round analysis. Namely, the expression profile of a specimen can be determined with a DNA chip. A DNA chip may comprise probes, which have been spotted thereon, to detect the expression level of the metagene-defining genes of the present invention, i.e. the genes described in Tables 1-8. A probe may be designed for each marker gene selected, and spotted on a DNA chip. Such a probe may be, for example, an oligonucleotide comprising 5-50 nucleotide residues. Methods for synthesizing such oligonucleotides on DNA chips are known to those skilled in the art. Longer DNAs can be synthesized by PCR or chemically. Methods for spotting long DNA, which is synthesized by PCR or the like, onto a glass slide are also known to those skilled in the art. A DNA chip that is obtained by the methods described above can be used for estimating the efficacy of a therapeutic agent in treating a subject afflicted with cancer according to the present invention.

DNA microarray and methods of analyzing data from microarrays are well-described in the art, including in DNA Microarrays: A Molecular Cloning Manual. Ed. by Bowtel and Sambrook (Cold Spring Harbor Laboratory Press, 2002); Microarrays for an Integrative Genomics by Kohana (MIT Press, 2002); A Biologist's Guide to Analysis of DNA Micraarray Data, by Knudsen (Wiley, John & Sons, Incorporated, 2002); DNA Microarrays: A Practical Approach, Vol. 205 by Schema (Oxford University Press, 1999); and Methods of Microarray Data Analysis II, ed. by Lin et al. (Kluwer Academic Publishers, 2002) all of which are incorporated herein by reference.

One aspect of the invention provides a kit comprising: (a) any of the gene chips described herein; and (b) one of the computer-readable mediums described herein.

In some embodiments, the arrays include probes for at least 2, 3, 4, 5, 6, 7, 8, 9, 10, 15, 20, 25, 30, 40, or 50 of the genes listed in one of Tables 1-8. In certain embodiments, the number of genes that are from one of the Tables that are represented on the array is at least 5, at least 10, at least 25, at least 50, at least 75 or more, including all of the genes listed in the table. Where the subject arrays include probes for additional genes not listed in the tables, in certain embodiments the number % of additional genes that are represented does not exceed about 50%, 40%, 30%, 20%, 15%, 10%, 8%, 6%, 5%, 4%, 3%, 2% or 1%. In some embodiments, a great majority of genes in the collection are genes that define the metagenes of the invention, whereby great majority is meant at least about 75%, usually at least about 80% and sometimes at least about 85, 90, 95% or higher, including embodiments where 100% of the genes in the collection are metagene-defining genes. In an alternative embodiment, the arrays for use in the invention may include a majority of probes that are not listed in any of Tables 1-8.

The kits of the subject invention may include the above described arrays or gene chips. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a remote site. Any convenient means of conveying instructions may be present in the kits.

The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber.

### Diagnostic Business Methods

One aspect of the invention provides methods of conducting a diagnostic business, including a business that provides a health care practitioner with diagnostic information for the treatment of a subject afflicted with cancer. One such method comprises one, more than one, or all of the following steps: (i) obtaining an tumor sample from the subject; (ii) determining the expression level of multiple genes in the sample; (iii) defining the value of one or more metagenes from the expression levels of step (ii), wherein each metagene is defined by extracting a single dominant value using single value decomposition (SVD) from a cluster of genes associated with sensitivity to an anti-cancer agent; (iv) averaging the predictions of one or more statistical tree models applied to the values, wherein each model includes one or more nodes, each node representing a meta gene, each node including a statistical predictive probability of sensitivity to an anti-cancer agent, wherein at least one metagene is one of metagenes 1-7; and (v) providing the health care practitioner with the prediction from step (iv).

In one embodiment, obtaining a tumor sample from the subject is effected by having an agent of the business (or a subsidiary of the business) remove a tumor sample from the subject, such as by a surgical procedure. In another embodiment, obtaining a tumor sample from the subject comprises receiving a sample from a health care practitioner, such as by shipping the sample, preferably frozen. In one embodiment, the sample is a cellular sample, such as a mass of tissue. In one embodiment, the sample comprises a nucleic acid sample, such as a DNA, cDNA, mRNA sample, or combinations thereof, which was derived from a cellular tumor sample from the subject. In one embodiment, the prediction from step (iv) is provided to a health care practitioner, to the patient, or to any other business entity that has contracted with the subject.

In one embodiment, the method comprises billing the subject, the subject's insurance carrier, the health care practitioner, or an employer of the health care practitioner. A government agency, whether local, state or federal, may also be billed for the services. Multiple parties may also be billed for the service.

In some embodiments, all the steps in the method are carried out in the same general location. In certain embodiments, one or more steps of the methods for conducting a diagnostic business are performed in different locations. In one embodiment, step (ii) is performed in a first location, and step (iv) is performed in a second location, wherein the first location is remote to the second location. The other steps may be performed at either the first or second location, or in other locations. In one embodiment, the first location is remote to the second location. A remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart In one embodiment, two locations that are remote relative to each other arc at least 1, 2, 3, 4, 5, 10, 20, 50, 100, 200, 500, 1000, 2000 or 5000 km apart. In another embodiment, the two locations are in different countries, where one of the two countries is the United States.

Some specific embodiments of the methods described herein where steps are performed in two or more locations comprise one or more steps of communicating information between the two locations. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In one specific embodiment, the method comprises one or more data transmission steps between the locations. In one embodiment, the data transmission step occurs via an electronic communication link, such as the internet. In one embodiment, the data transmission step from the first to the second location comprises experimental parameter data, such as the level of gene expression of multiple genes. In some embodiments, the data transmission step from the second location to the first location comprises data transmission to intermediate locations. In one specific embodiment, the method comprises one or more data transmission substeps from the second location to one or more intermediate locations and one or more data transmission substeps from one or more intermediate locations to the first location, wherein the intermediate locations are remote to both the first and second locations. In another embodiment, the method comprises a data transmission step in which a result from gene expression is transmitted from the second location to the first location.

In one embodiment, the methods of conducting a diagnostic business comprise the step of determining if the subject carries an allelic form of a gene whose presence correlates to sensitivity or resistance to a chemotherapeutic agent. This may be achieved by analyzing a nucleic acid sample from the patient and determining the DNA sequence of the allele. Any technique known in the art for determining the presence of mutations or polymorphisms may be used. ‘The method is not limited to any particular mutation or to any particular allele or gene. For example, mutations in the epidermal growth factor receptor (EGFR) gene are found in human lung adenocarcinomas and are associated with sensitivity to the tyrosine kinase inhibitors gefitinib and erlotinib. (See, e.g., Yi et al. Proc Natl Acad Sci USA. 2006 May 16; 103(20):7817-22; Shimato et al. Neuro-oncol. 2006 April; 8(2): 137-44). Similarly, mutations in breast cancer resistance protein (HCRP) modulate the resistance of cancer cells to BCRP-substrate anticancer agents (Yanase et al., Cancer Lett. 2006 Mar. 8; 234(1):73-80).

### Computer Readable Media Comprising Gene Expression Profiles

The invention also contemplates computer readable media that comprises gene expression profiles. Such media can contain all or part of the gene expression profiles of the genes listed in the Tables that comprise the responsivity predictor set. The media can be a list of the genes or contain the raw data for running a user's own statistical calculation, such as the methods disclosed herein.

Another aspect of the invention provides a program product (i.e., software product) for use in a computer device that executes program instructions recorded in a computer-readable medium to perform one or more steps of the methods described herein, such for estimating the efficacy of a therapeutic agent in treating a subject afflicted with cancer.

One aspect of the invention provides a computer readable medium having computer readable program codes embodied therein, the computer readable medium program codes performing one or more of the following functions: defining the value of one or more metagenes from the expression levels of genes in known responsive and sensitive cells; defining a metagene value by extracting a single dominant value using singular value decomposition (SVD) from a cluster of genes associated with tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

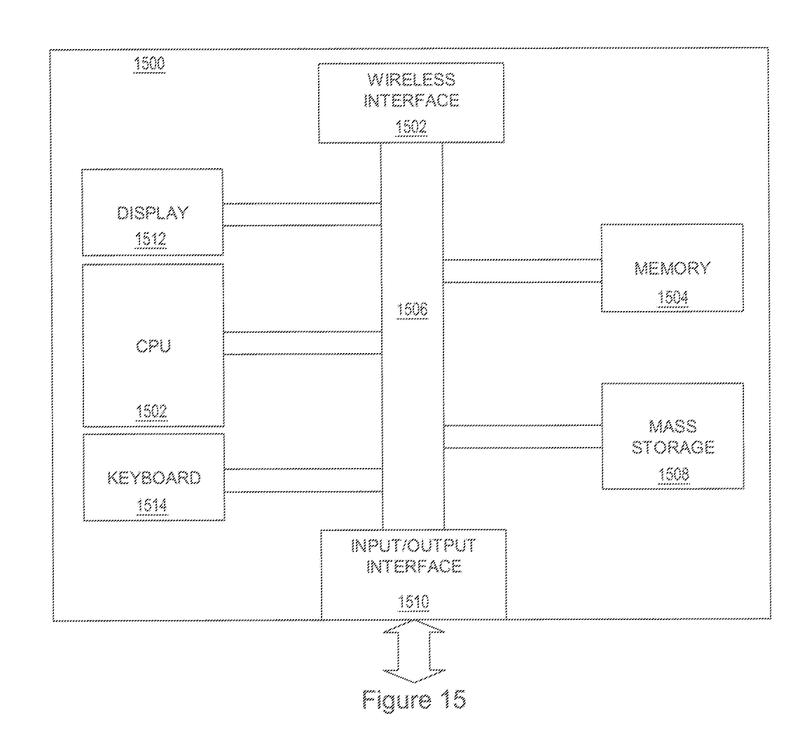

Another related aspect of the invention provides kits comprising the program product or the computer readable medium, optionally with a computer system. One aspect of the invention provides a system, the system comprising: a computer (See FIG. 15); a computer readable medium, operatively coupled to the computer, the computer readable medium program codes performing one or more of the following functions: defining the value of one or more metagenes from the expression levels genes; defining a metagene value by extracting a single dominant value using singular value decomposition (SVD) from a cluster of genes associated tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

In one embodiment, the program product comprises: a recordable medium; and a plurality of computer-readable instructions executable by the computer device to analyze data from the array hybridization steps, to transmit array hybridization from one location to another, or to evaluate genome-wide location data between two or more genomes. Computer readable media include, but are not limited to, CD-ROM disks (CD-R, CD-RW), DVD-RAM disks, DVD-RW disks, floppy disks and magnetic tape.

A related aspect of the invention provides kits comprising the program products described herein. The kits may also optionally contain paper and/or computer-readable format instructions and/or information, such as, but not limited to, information on DNA microarrays, on tutorials, on experimental procedures, on reagents, on related products, on available experimental data, on using kits, on chemotherapeutic agents including their toxicity, and on other information. The kits optionally also contain in paper and/or computer-readable format information on minimum hardware requirements and instructions for running and/or installing the software. The kits optionally also include, in a paper and/or computer readable format, information on the manufacturers, warranty information, availability of additional software, technical services information, and purchasing information. The kits optionally include a video or other viewable medium or a link to a viewable format on the internet or a network that depicts the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of data, as well as the transmission of data steps, can be implemented by the use of one or more computer systems. Computer systems are readily available. The processing that provides the displaying and analysis of image data for example, can be performed on multiple computers or can be performed by a single, integrated computer or any variation thereof. The components contained in the computer system are those typically found in general purpose computer systems used as servers, workstations, personal computers, network terminals, and the like. See FIG. 15. In fact, these components are intended to represent a broad category of such computer components that are well known in the art.

FIG. 15 shows a functional block diagram of general purpose computer system 1500 for performing the functions of the software according to an illustrative embodiment of the invention. The exemplary computer system 1500 includes a central processing unit (CPU) 3002, a memory 1504, and an interconnect bus 1506. The CPU 1502 may include a single microprocessor or a plurality of microprocessors for configuring computer system 1500 as a multi-processor system. The memory 1504 illustratively includes a main memory and a read only memory. The computer 1500 also includes the mass storage device 1508 having, for example, various disk drives, tape drives, etc. The main memory 1504 also includes dynamic random access memory (DRAM) and high-speed cache memory. In operation, the main memory 1504 stores at least portions of instructions and data for execution by the CPU 1502.

The mass storage 1508 may include one or more magnetic disk or tape drives or optical disk drives, for storing data and instructions for use by the CPU 1502. At least one component of the mass storage system 1508, preferably in the form of a disk drive or tape drive, stores one or more databases, such as databases containing of transcriptional start sites, genomic sequence, promoter regions, or other information.

The mass storage system 1508 may also include one or more drives for various portable media, such as a floppy disk, a compact disc read only memory (CD-ROM), or an integrated circuit non-volatile memory adapter (i.e., PC-MCIA adapter) to input and output data and code to and from the computer system 1500.

The computer system 1500 may also include one or more input/output interfaces for communications, shown by way of example, as interface 1510 for data communications via a network. The data interface 1510 may be a modem, an Ethernet card or any other suitable data communications device. To provide the functions of a computer system according to FIG. 15 the data interface 1510 may provide a relatively high-speed link to a network, such as an intranet, internet, or the Internet, either directly or through an another external interface. The communication link to the network may be, for example, optical, wired, or wireless (e.g., via satellite or cellular network). Alternatively, the computer system 1500 may include a mainframe or other type of host computer system capable of Web-based communications via the network.

The computer system 1500 also includes suitable input/output ports or use the interconnect bus 1506 for interconnection with a local display 1512 and keyboard 1514 or the like serving as a local user interface for programming and/or data retrieval purposes. Alternatively, server operations personnel may interact with the system 1500 for controlling and/or programming the system from remote terminal devices via the network.

The following examples are provided to illustrate aspects of the invention but are not intended to limit the invention in any manner.

### EXAMPLES

### Example 1

### A Gene Expression Based Predictor of Sensitivity to Docetaxel

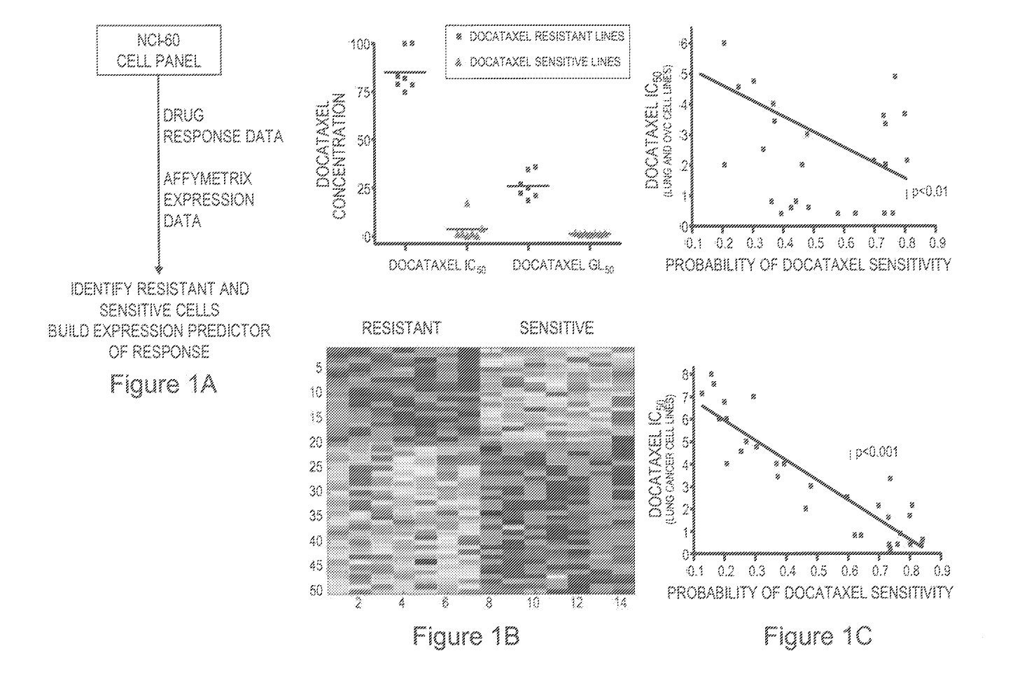

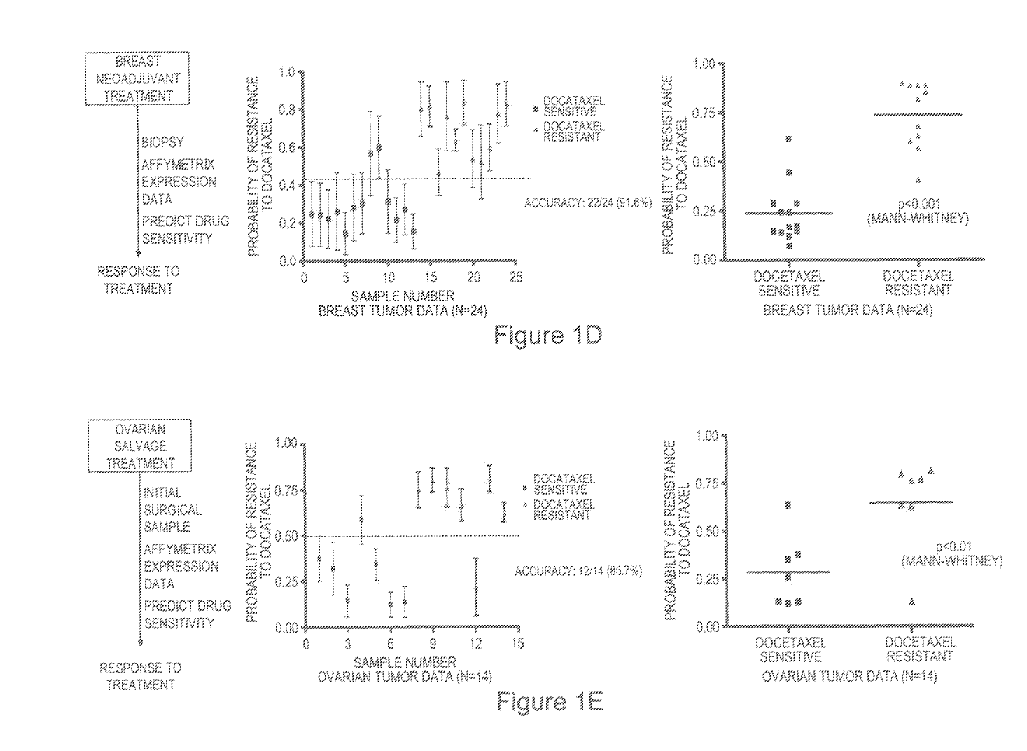

The NCI-60 panel⁴⁹ was used to develop predictors of chemotherapeutic drug response, and cell lines that were most resistant or sensitive to docetaxel were identified (FIG. 1A, B). Genes whose expression most highly correlated with drug sensitivity, using Bayesian binary regression analysis, were selected to develop a model that differentiates a pattern of docetaxel sensitivity from resistance. A gene expression signature consisting of 50 genes was identified that classified on the basis of docetaxel sensitivity (FIG. 1 B, bottom panel).

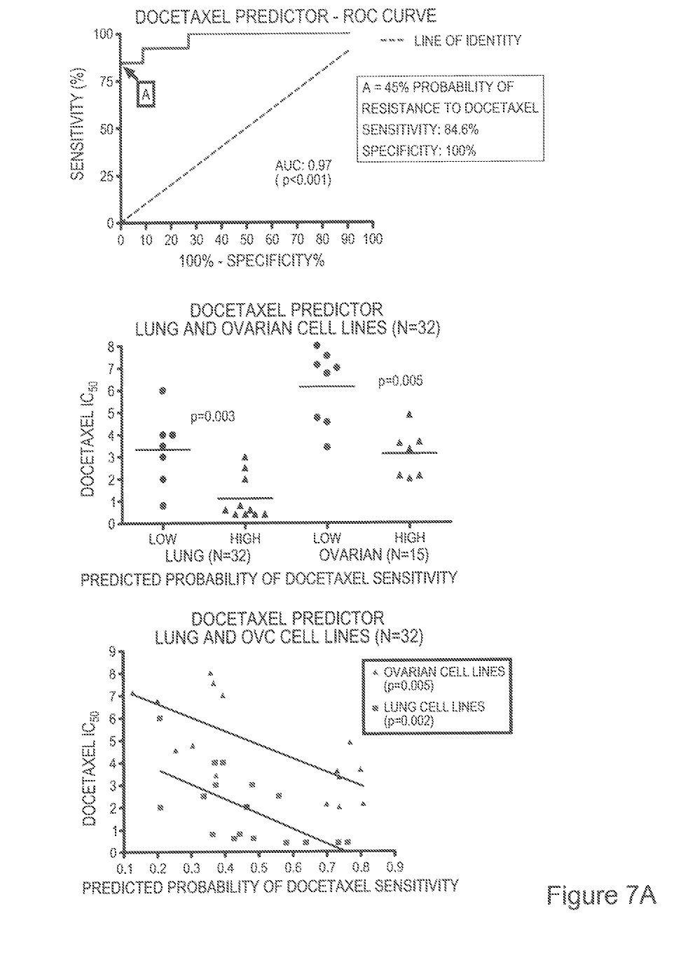

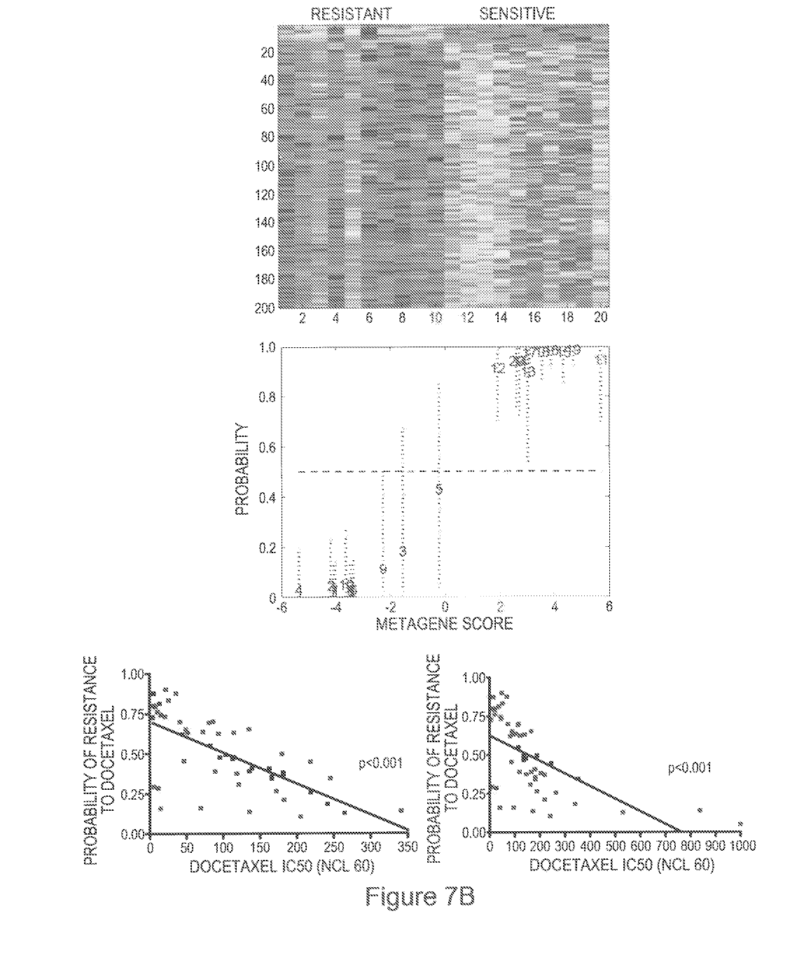

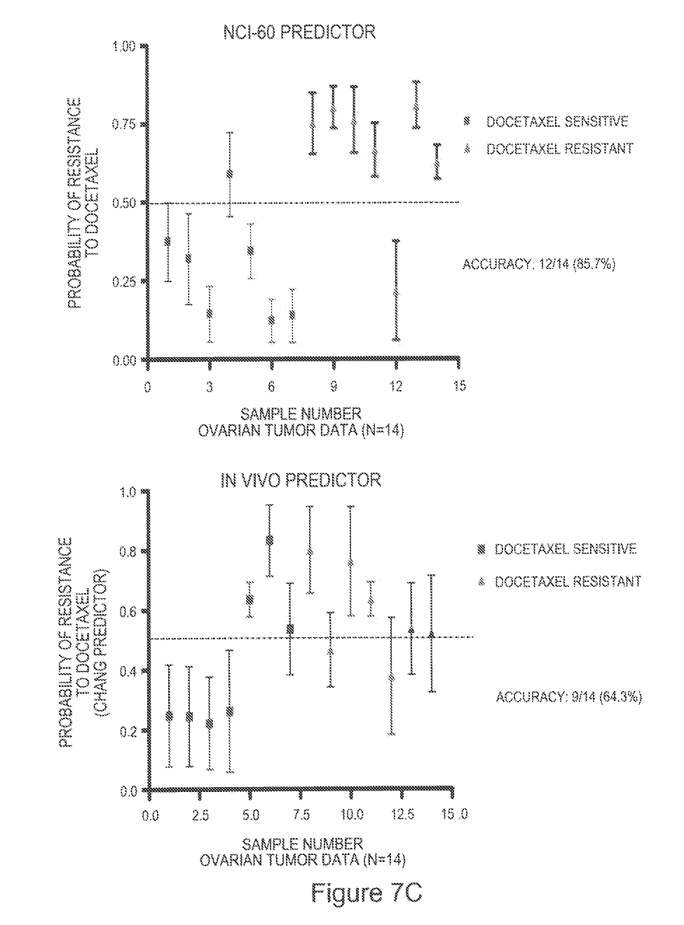

In addition to leave-one-out cross validation, we utilized an independent dataset derived from docetaxel sensitivity assays in a series of 30 lung and ovarian cancer cell lines for further validation. As shown in FIG. 1C (top panel), the correlation between the predicted probability of sensitivity to docetaxel (in both lung and ovarian cell lines) and the respective IC50 for docetaxel confirmed the capacity of the docetaxel predictor to predict sensitivity to the drug in cancer cell lines (FIG. 7). In each case, the accuracy exceeded 80%. Finally, a second independent dataset including 29 lung cancer cell lines (Gemma A, GEO accession number: GSE 4127), was used to predict and measure docetaxel sensitivity. As shown in FIG. 1C (bottom panel), the docetaxel sensitivity model developed from the NCI-60 panel again predicted sensitivity in this independent data set, again with an accuracy exceeding 80%.

### Example 2

### Utilization of the Expression Signature to Predict Docetaxel Response in Patients

The development of a gene expression signature capable of predicting in vitro docetaxel sensitivity provides a tool that might be useful in predicting response to the drug in patients. We made use of published studies with clinical and genomic data that linked gene expression data with clinical response to docetaxel in a breast cancer neoadjuvant study⁵⁰ (FIG. 1D) to test the capacity of the in vitro docetaxel sensitivity predictor to accurately identify those patients that responded to docetaxel. Using a 0.45 predicted probability of response as the cut-off for predicting positive response, as determined by ROC curve analysis (FIG. 7A), the in vitro generated profile correctly predicted docetaxel response in 22 out of 24 patient samples, achieving an overall accuracy of 91.6% (FIG. 1D). Applying a Mann-Whitney U test for statistical significance demonstrates the capacity of the predictor to distinguish resistant from sensitive patients (FIG. 1D, right panel). We extended this further by predicting the response to docetaxel as salvage therapy for ovarian cancer. As shown in FIG. 1E, the prediction of response to docetaxel in patients with advanced ovarian cancer achieved an accuracy exceeding 85% (FIG. 1E, middle panel). Further, an analysis of statistical significance demonstrated the capacity of the predictors to distinguish patients with resistant versus sensitive disease (FIG. 1E, right panel).

We also performed a complementary analysis using the patient response data to generate a predictor and found that the in vivo generated signature of response predicted sensitivity of NCI-60 cell lines to docetaxel (FIG. 7B). This crossover is further emphasized by the fact that the genes represented in either the initial in vitro generated docetaxel predictor or the alternative in vivo predictor exhibit considerable overlap. (Table 4). We also note that the predictor of docetaxel sensitivity developed from the NCI-60 data was more accurate in predicting patient response in the ovarian samples than the predictor developed from the breast neoadjuvant patient data (85.7% vs. 64.3%) (FIG. 7C).

### Example 3

### Development of a Panel of Gene Expression Signatures that Predict Sensitivity to Chemotherapeutic Drugs

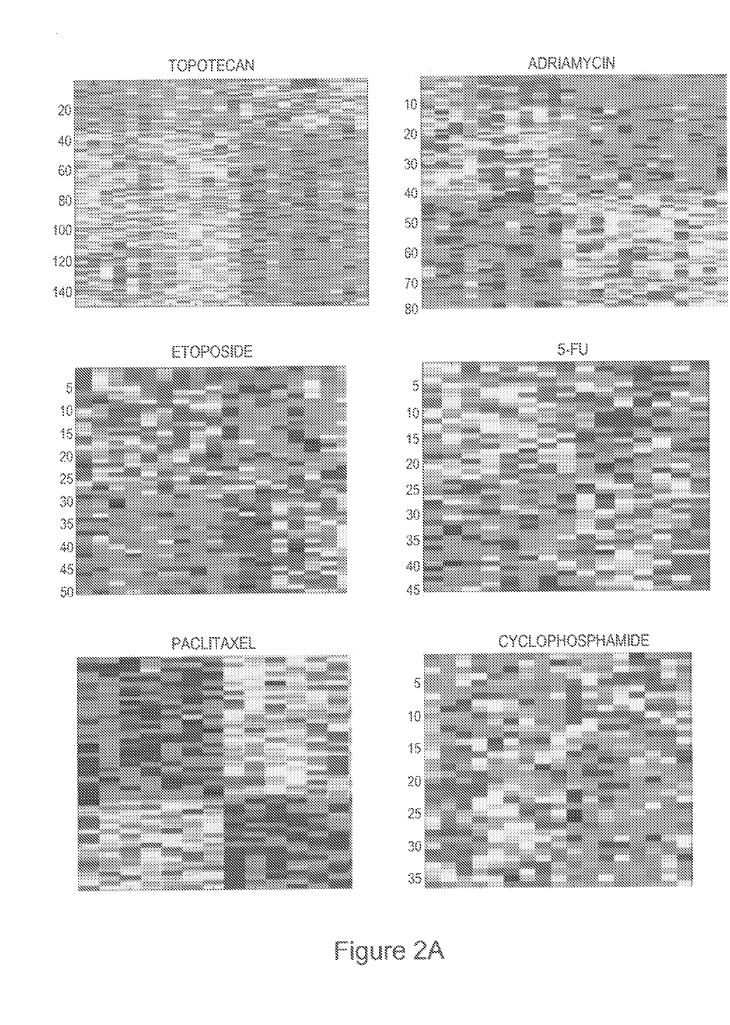

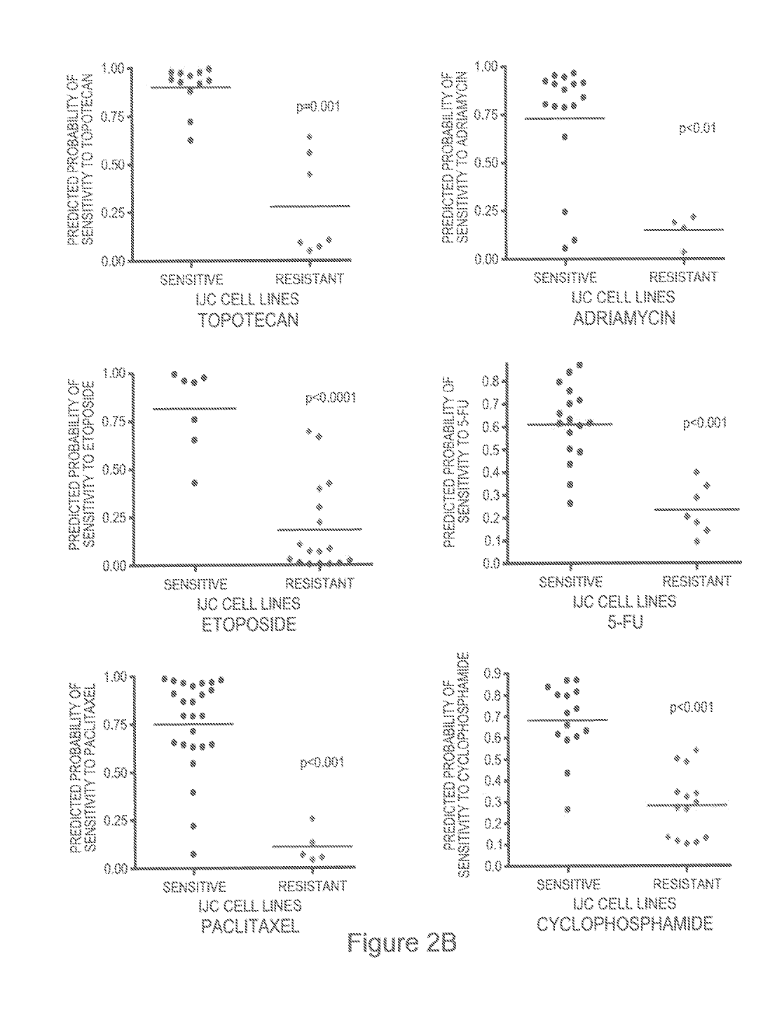

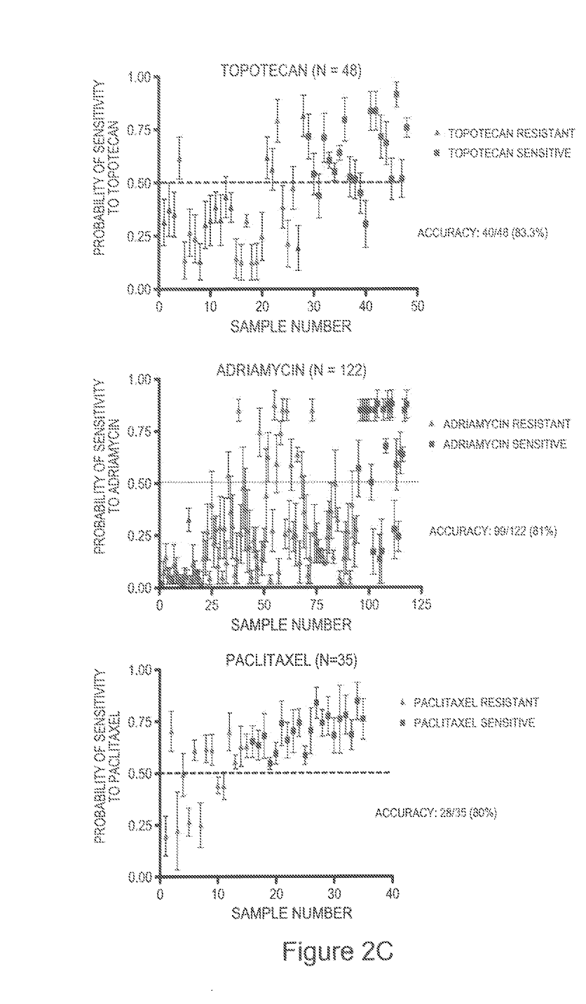

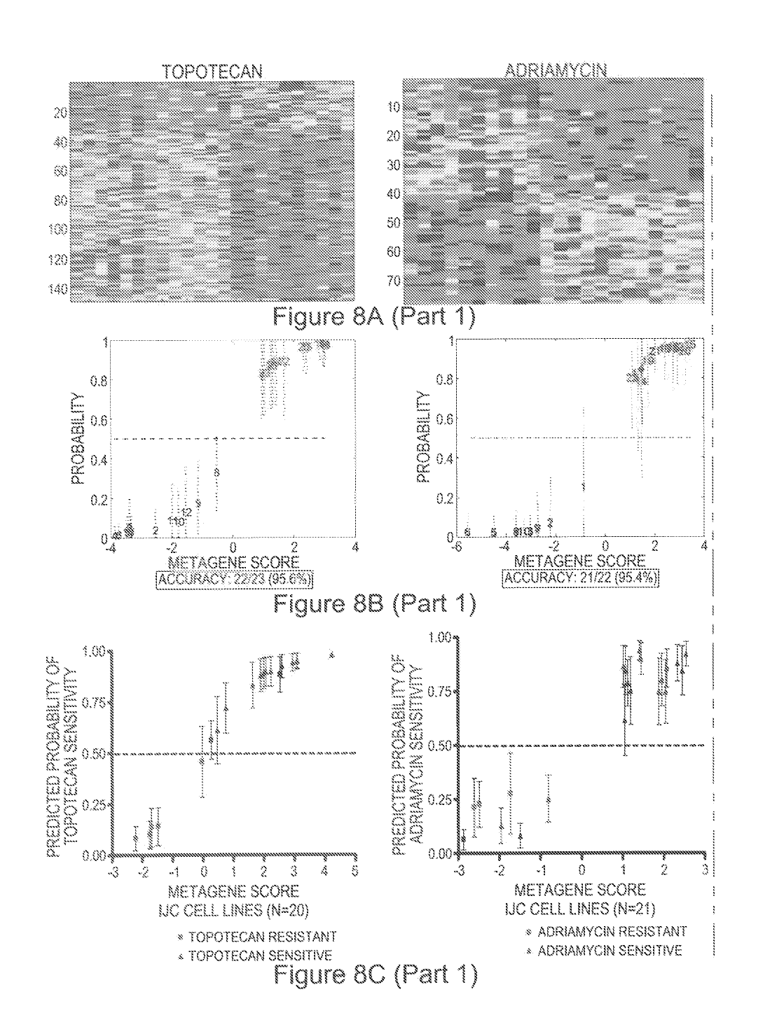

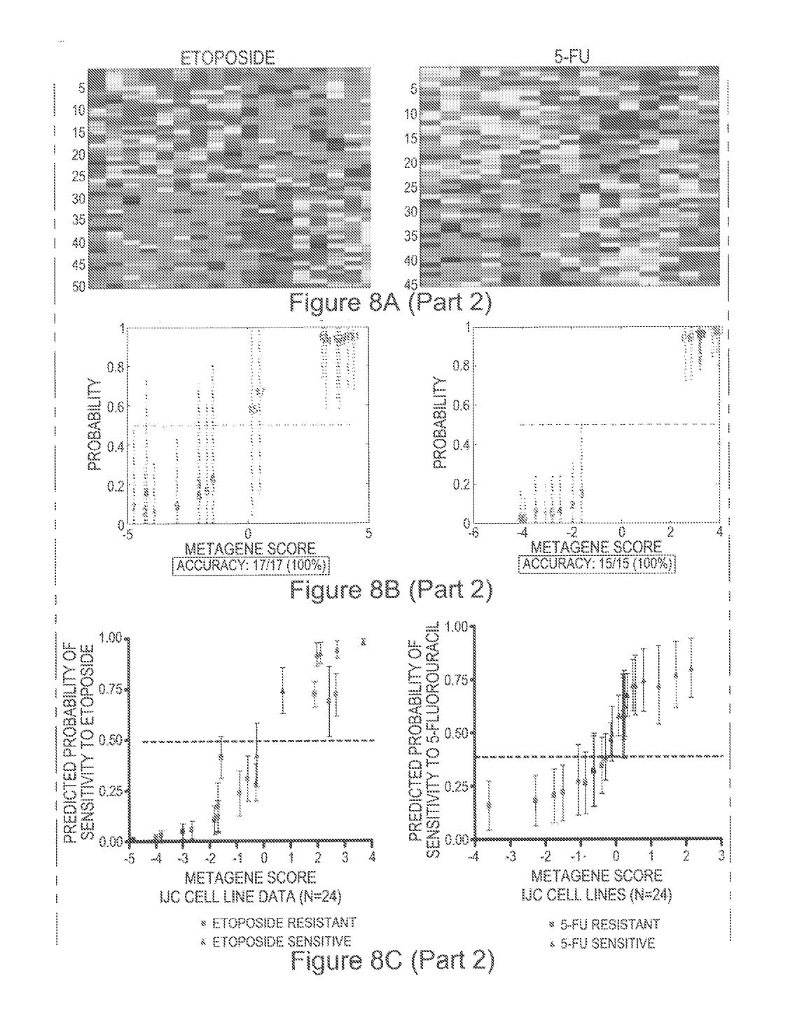

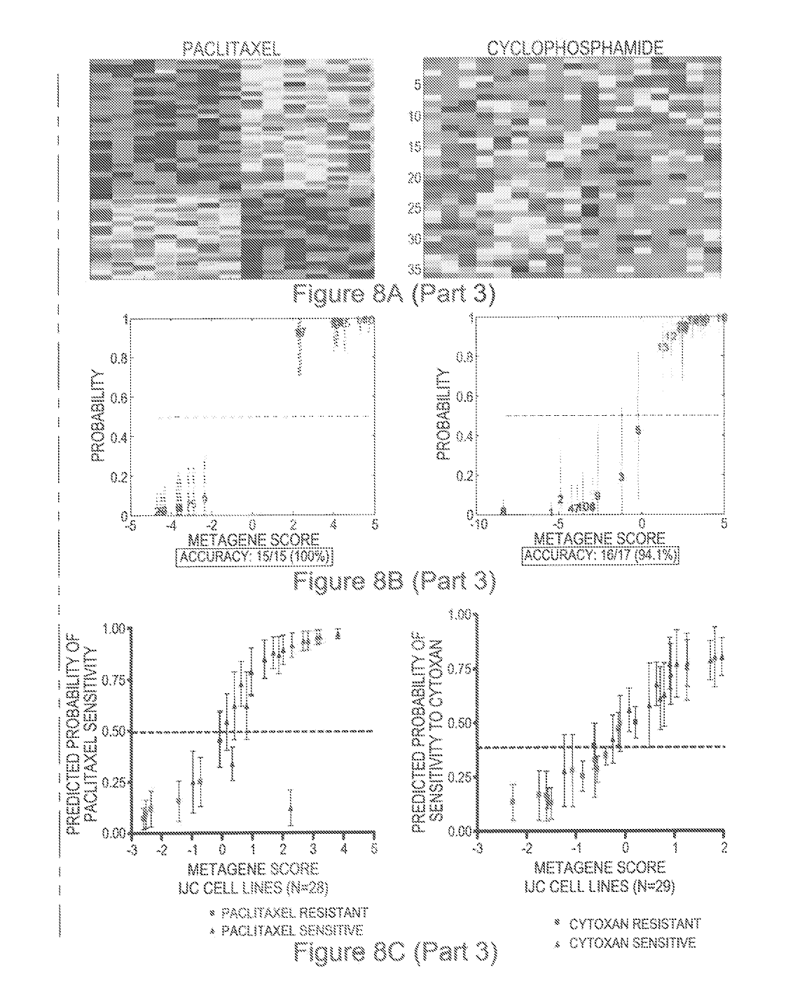

Given the development of a docetaxel response predictor, we examined the NCI-60 data set for other opportunities to develop predictors of chemotherapy response. Shown in FIG. 2A are a series of expression profiles developed from the NCI-60 dataset that predict response to topotecan, adriamycin, etoposide, 5-fluorouracil (5-FU), taxol (paclitaxel), and cyclophosphamide (cytotoxan). In each case, the leave-one-out cross validation analyses demonstrate a capacity of these profiles to accurately predict the samples utilized in the development of the predictor (FIG. 8B). Each profile was then further validated using in vitro response data from independent datasets; in each case, the profile developed from the NCI-60 data was capable of accurately (>85%) predicting response in the separate dataset of approximately 30 cancer cell lines for which the dose response information and relevant Affymetrix U133A gene expression data is publicly available³⁷ (FIG. 8C) and Table 16). Once again, applying a Mann-Whitney U test for statistical significance demonstrates the capacity of the predictor to distinguish resistant from sensitive patients (FIG. 2B).

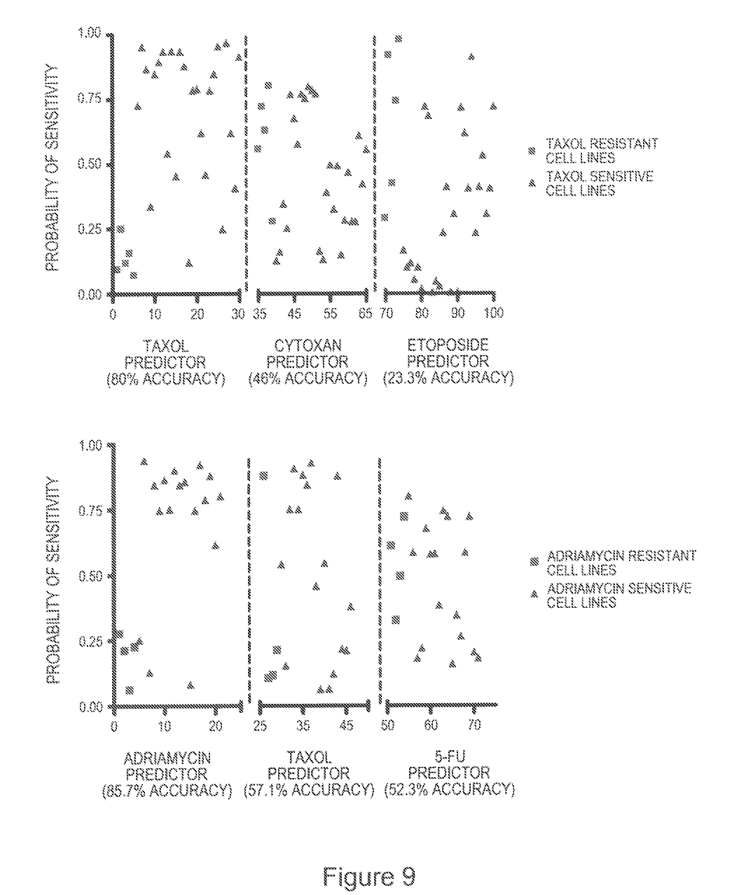

In addition to the capacity of each signature to distinguish cells that are sensitive or resistant to a particular drug, we also evaluated the extent to which a signature was also specific for an individual chemotherapeutic agent. From the example shown in FIG. 9, using the validations of chemosensitivity seen in the independent European (UC) cell line data it is clear that each of the signatures is specific for the drug that was used to develop the predictor. In each case, individual predictors of response to the various cytotoxic drugs was plotted against cell lines known to be sensitive or resistant to a given chemotherapeutic agent (e.g., adriamycin, paclitaxel).

Given the ability of the in vitro developed gene expression profiles to predict response to docetaxel in the clinical samples, we extended this approach to test the ability of additional signatures to predict response to commonly used salvage therapies for ovarian cancer and an independent data set of samples from adriamycin treated patients (Evans W, GSE650, GSE651). As shown in FIG. 2C, each of these predictors was capable of accurately predicting the response to the drugs in patient samples, achieving an accuracy in excess of 81% overall. In each case, the positive and negative predictive values confirm the validity and clinical utility of the approach (Table 16).

### Example 4

### Chemotherapy Response Signatures Predict Response to Multi-Drug Regimens

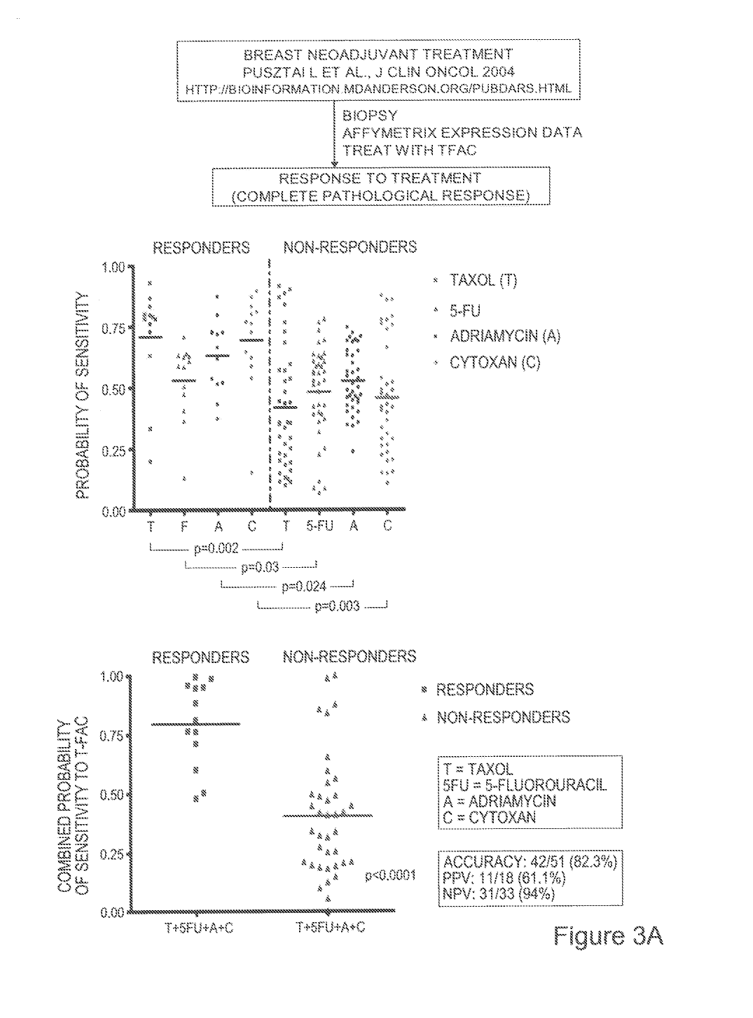

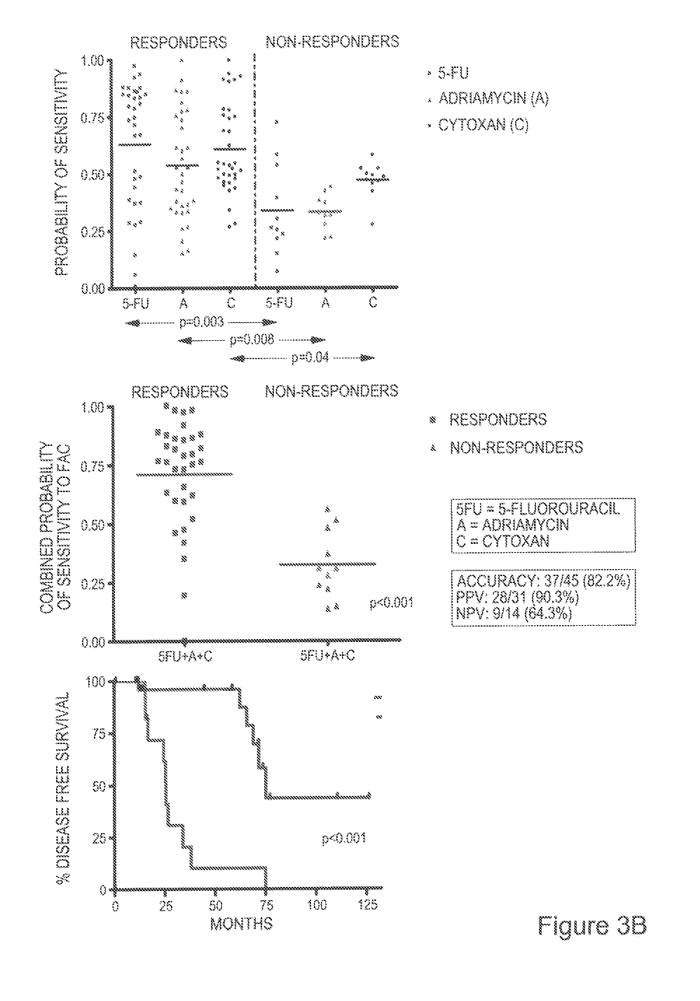

Many therapeutic regimens make use of combinations of chemotherapeutic drugs raising the question as to the extent to which the signatures of individual therapeutic response will also predict response to a combination of agents. To address this question, we have made use of data from a breast neoadjuvant treatment that involved the use of paclitaxel, 5-fluorouracil, adriamycin, and cyclophosphamide (TFAC)^{55,56} (FIG. 3A). Using available data from the 51 patients to then predict response with each of the single agent signatures (paclitaxel, 5-FU, adriamycin and cyclophosphamide) developed from the NCI-60 cell line analysis; we then compared to the clinical outcome information which was represented as complete pathologic response. As shown in FIG. 3A (middle panel), the predicted response based on each of the individual chemosensitivity signatures indicated a significant distinction between the responders (n=13) and non-responders (n=38) with the exception of 5-fluorouracil. Importantly, the combined probability of sensitivity to the four agents in this TFAC neoadjuvant regimen was calculated using the probability theorem and it is clear from this analysis that the prediction of response based on a combined probability of sensitivity, built from the individual chemosensitivity predictions yielded a statistically significant (p<0.0001, Mann Whitney U) distinction between the responders and non-responders (FIG. 3A, bottom panel).

As a further validation of the capacity to predict response to combination therapy, we made use of gene expression data generated from a collection of breast cancer (n=45) samples from patients who received 5-fluorouracil, adriamycin and cyclophosphamide (FAC) in the adjuvant chemotherapy set. As shown in FIG. 3B (top panel), the predicted response based on signatures for 5-FU, adriamycin, and cyclophosphamide indicated a significant distinction between the responders (n=34) and non-responders (n=11) for each of the single agent predictors. Furthermore, the combined probability of sensitivity to the three agents in the FAC regimen was calculated and shown in the middle panel of FIG. 3B. It is evident from this analysis that the prediction of response based on a combined probability of sensitivity to the FAC regimen yielded a clear, significant (p<0.001, Mann Whitney U) distinction between the responders and non-responders (accuracy: 82.2%, positive predictive value: 90.3%, negative predictive value: 64.3%). We note that while it is difficult to interpret the prediction of clinical response in the adjuvant setting since many of these patients were likely free of disease following surgery, the accurate identification of non-responders is a clear endpoint that does confirm the capacity of the signatures to predict clinical response.

As a further measure of the relevance of the predictions, we examined the prognostic significance of the ability to predict response to FAC. As shown in FIG. 3B (bottom panel), there was a clear distinction in the population of patients identified as sensitive or resistant to FAC, as measured by disease-free survival (sensitive=blue, resistant=red). These results, taken together with the accuracy of prediction of response in the neoadjuvant setting where clinical endpoints are uncomplicated by confounding variables such as prior surgery, and results of the single agent validations, leads us to conclude that the signatures of chemosensitivity generated from the NCI-60 panel do indeed have the capacity to predict therapeutic response in patients receiving either single agent or combination chemotherapy (Table 17).

When comparing individual genes that constitute the predictors, it was interesting to observe that the gene coding for MAP-Tau, described previously as a determinant of paclitaxel sensitivity,⁵⁶ was also identified as a discriminator gene in the paclitaxel predictor generated using the NCI-60 data. Although, similar to the docetaxel example described earlier, a predictor for TFAC chemotherapy developed using the NCI-60 data was superior to the ability of the MAP-Tau based predictor described by Pusztai et al (Table 18).

### Example 5

### Patterns of Predicted Chemotherapy Response Across a Spectrum of Tumors

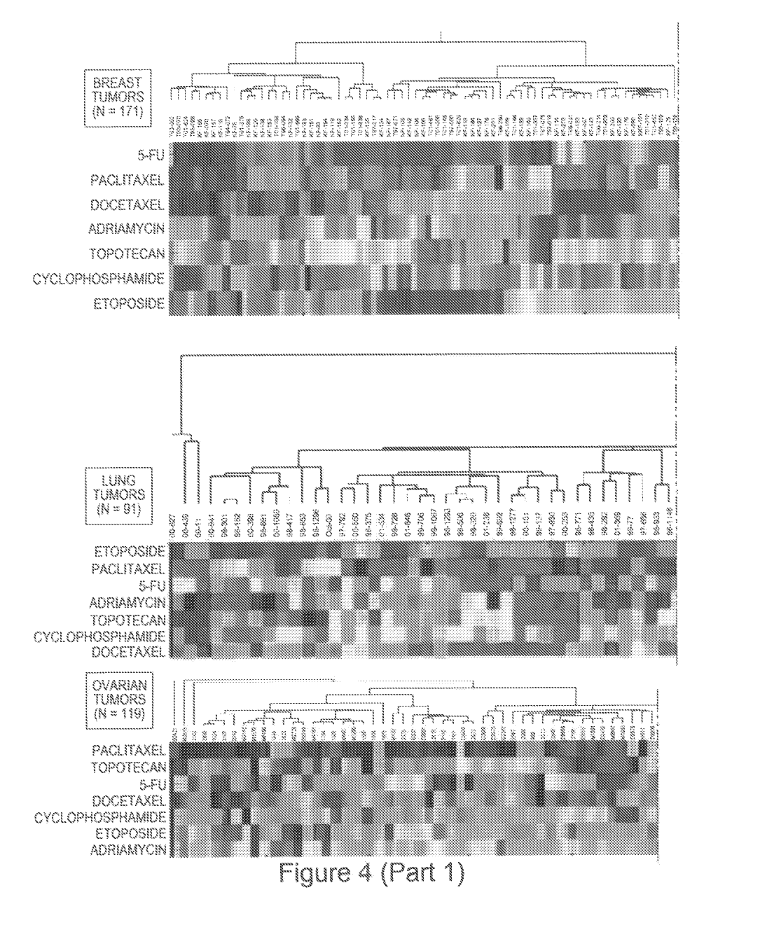

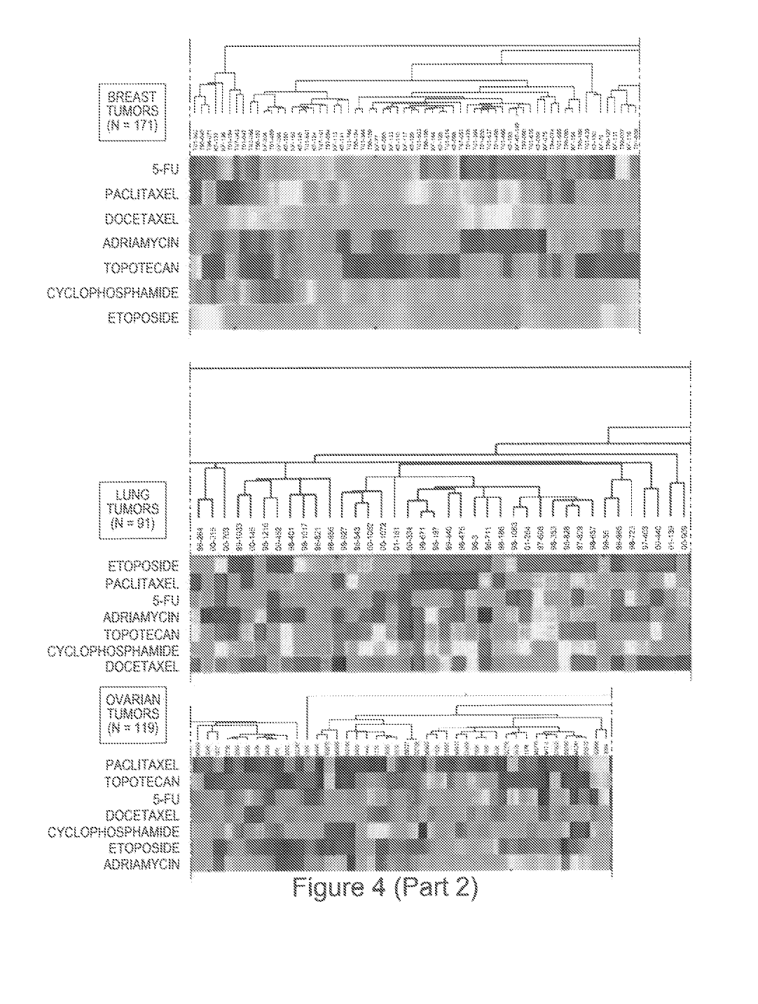

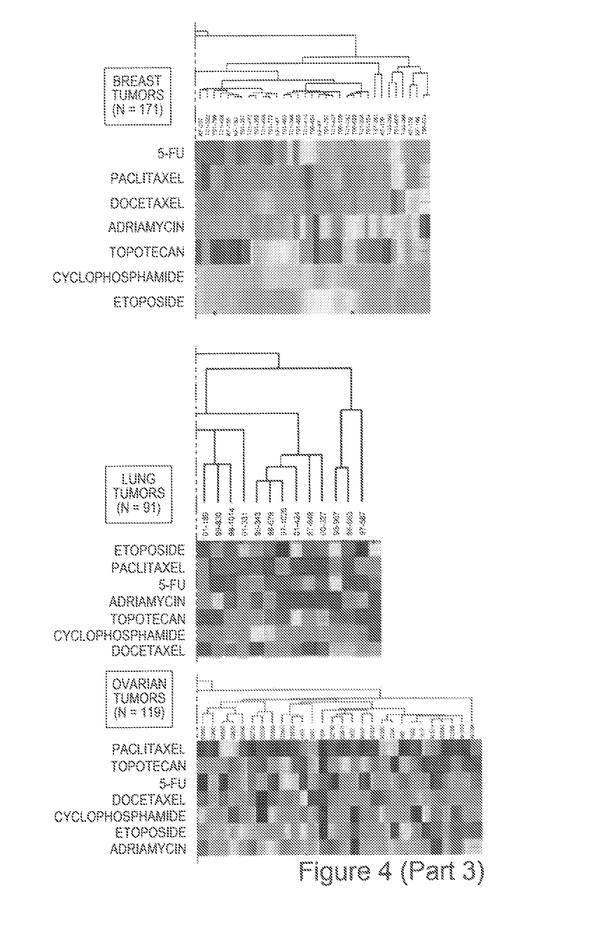

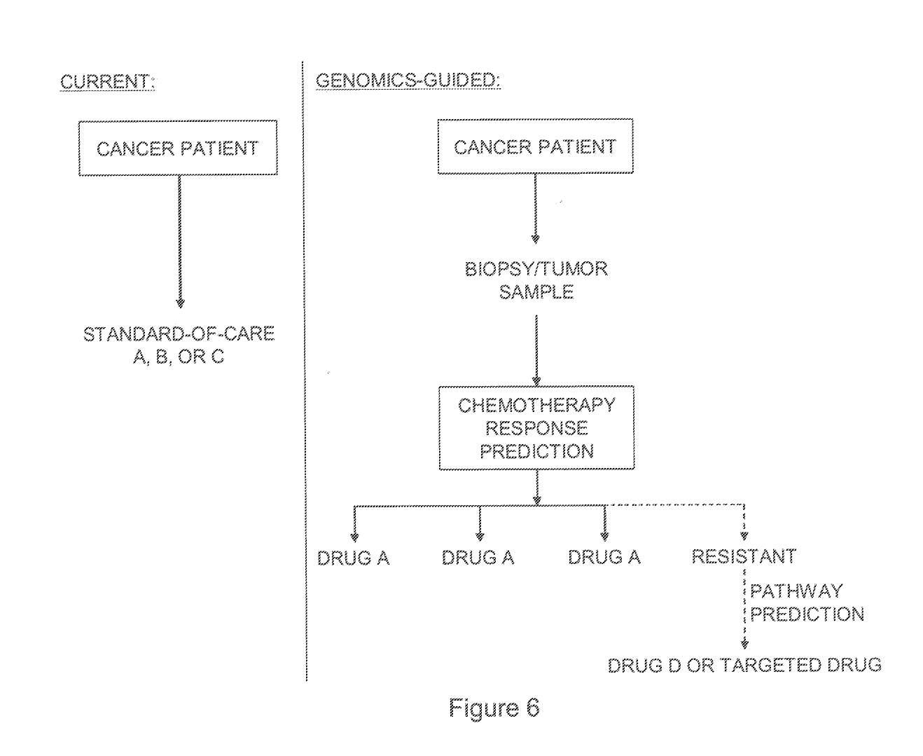

The availability of genomic-based predictors of chemotherapy response could potentially provide an opportunity for a rational approach to selection of drugs and combinations of drugs. With this in mind, we have utilized the panel of chemotherapy response predictors described in FIG. 6 to profile the potential options for use of these agents, by predicting the likelihood of sensitivity to the agents in a large collection of breast, lung, and ovarian tumor samples. We then clustered the samples according to patterns of predicted sensitivity to the various chemotherapeutics, and plotted a heatmap in which high probability of sensitivity response is indicated by red and low probability or resistance is indicated by blue (FIG. 4).

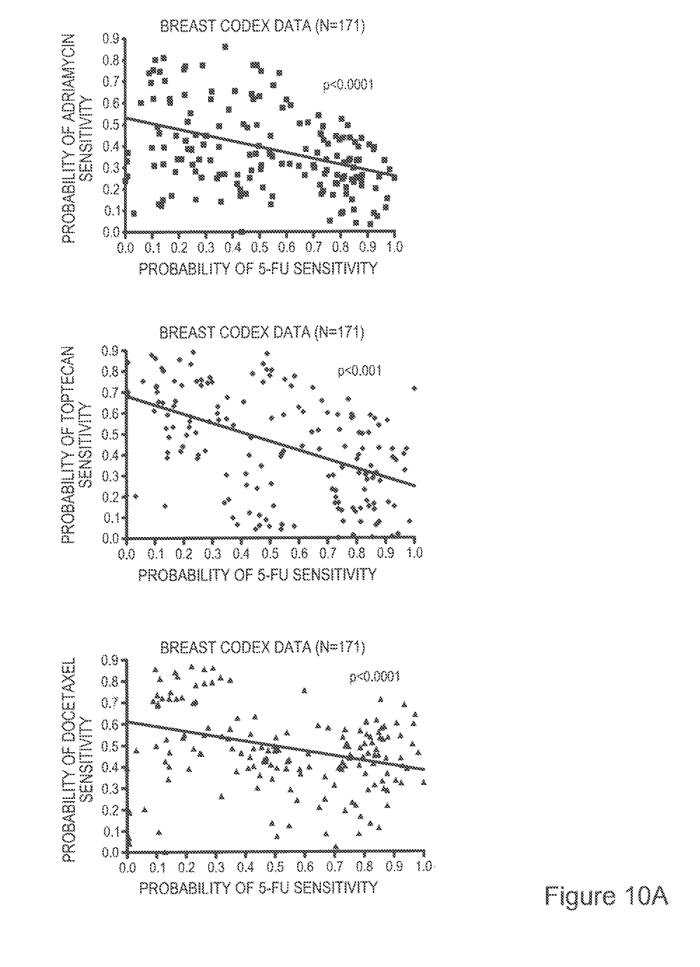

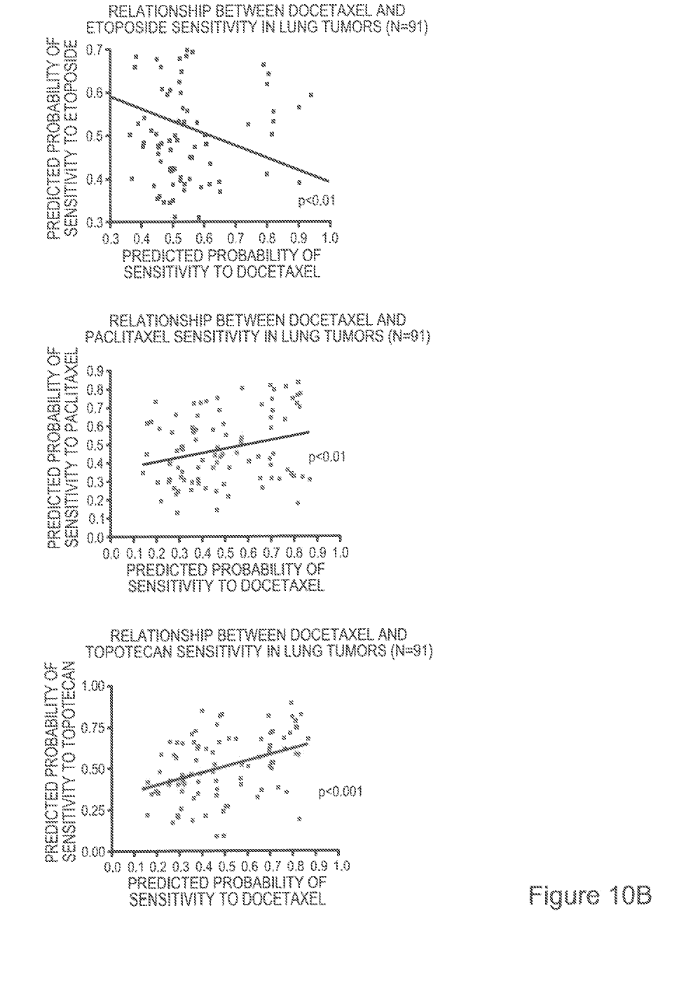

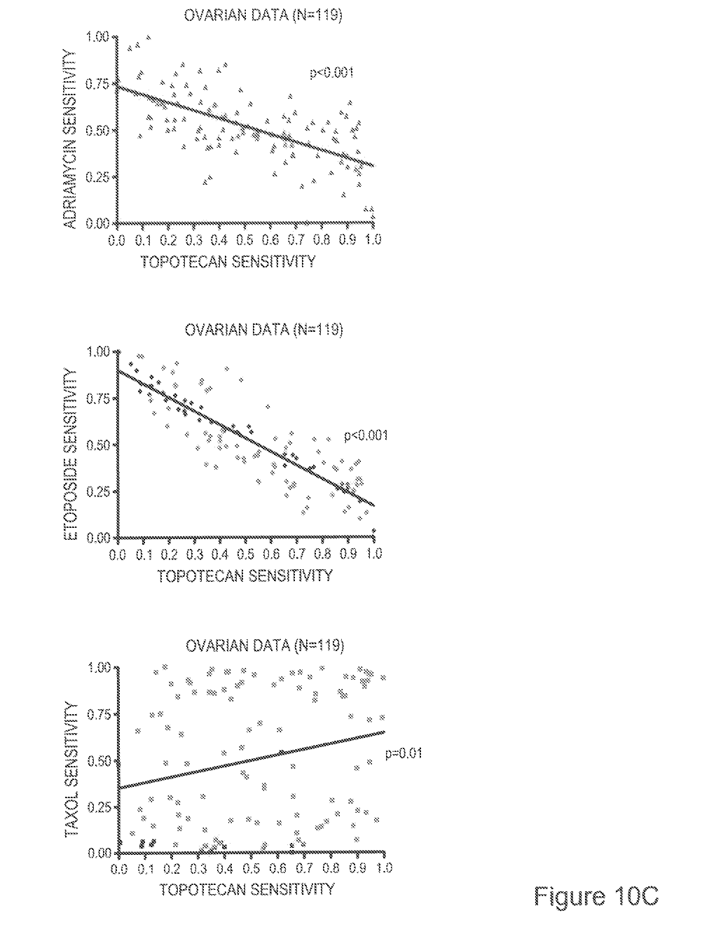

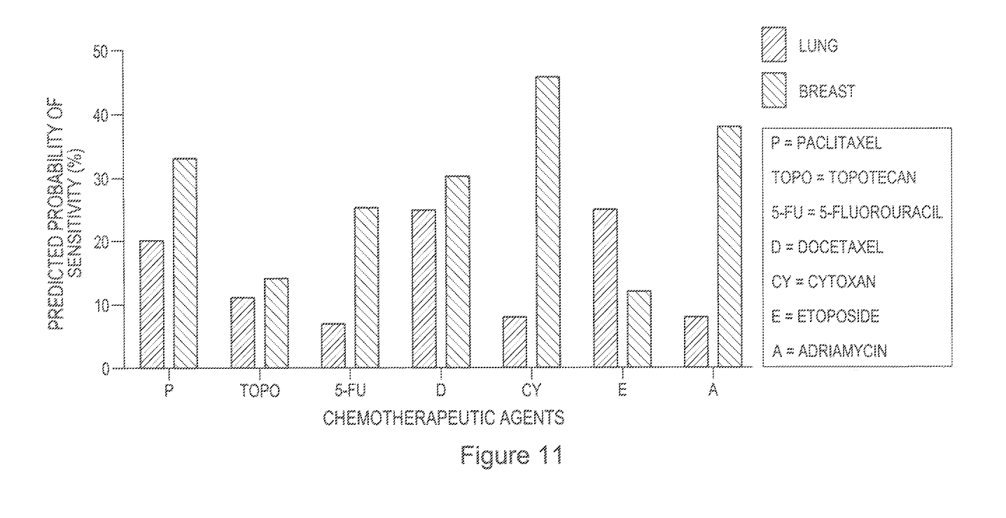

There are clearly evident patterns of predicted sensitivity to the various agents. In many cases, the predicted sensitivities to the chemotherapeutic agents are consistent with the previously documented efficacy of single agent chemotherapies in the individual tumor types⁵⁷. For instance, the predicted response rate for etoposide, adriamycin, cyclophosphamide, and 5-FU approximate the observed response for these single agents in breast cancer patients (FIG. 11). Likewise, the predicted sensitivity to etoposide, docetaxel, and paclitaxel approximates the observed response for these single agents in lung cancer patients (FIG. 11). This analysis also suggests possibilities for alternate treatments. As an example, it would appear that breast cancer patients likely to respond to 5-fluorouracil are resistant to adriamycin and docetaxel (FIG. 10A). Likewise, in lung cancer, docetaxel sensitive populations are likely to be resistant to etoposide (FIG. 10B). This is a potentially useful observation considering that both etoposide and docetaxel are viable front-line options (in conjunction with cis/carboplatin) for patients with lung cancer⁵⁸ A similar relationship is seen between topotecan and adriamycin, both agents used in salvage chemotherapy for ovarian cancer (FIG. 10C). Thus, by identifying patients/patient cohorts resistant to certain standard of care agents, one could avoid the side effects of that agent (e.g. topotecan) without compromising patient outcome, by choosing an alternative standard of care (e.g., adriamycin).

### Example 6

### Linking Predictions of Chemotherapy Sensitivity to Oncogenic Pathway Deregulation

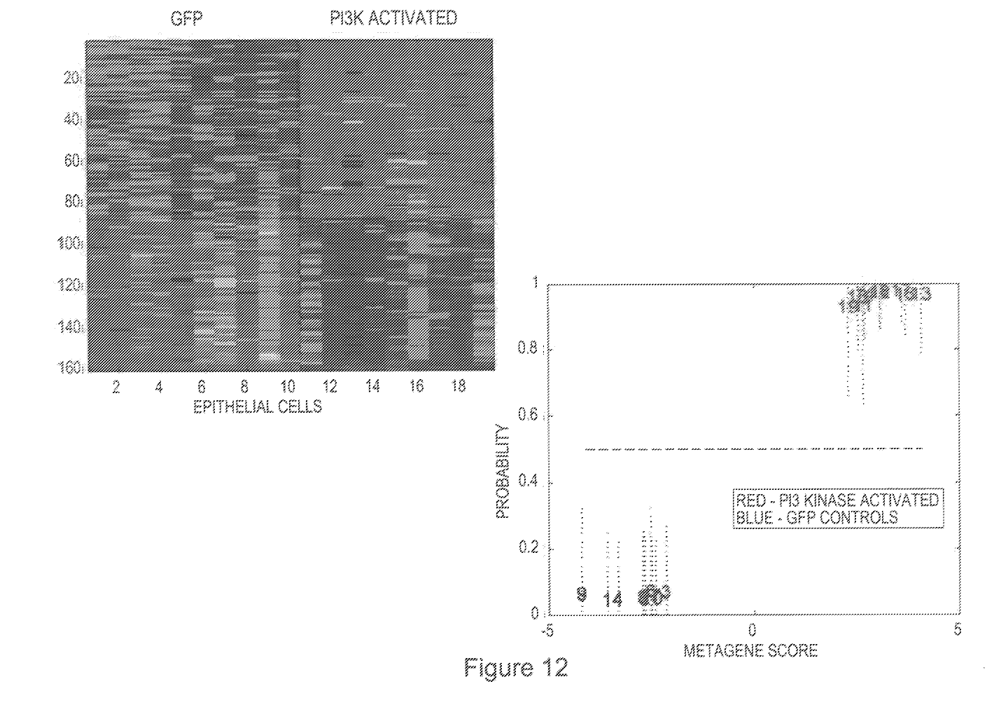

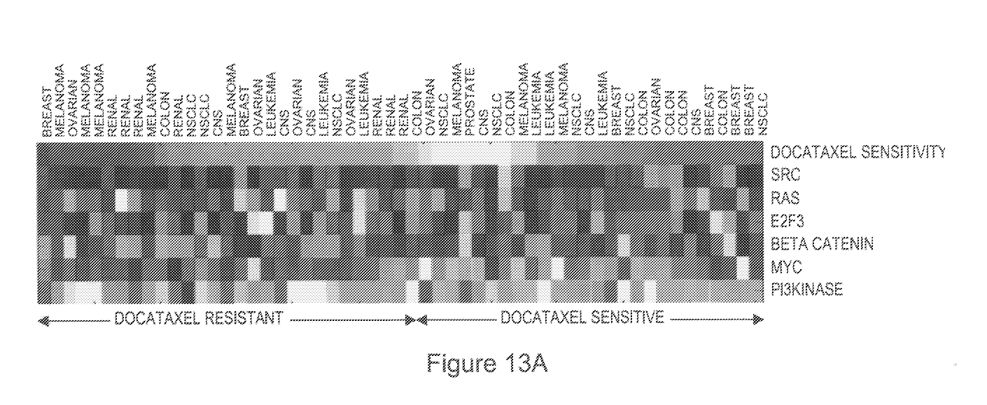

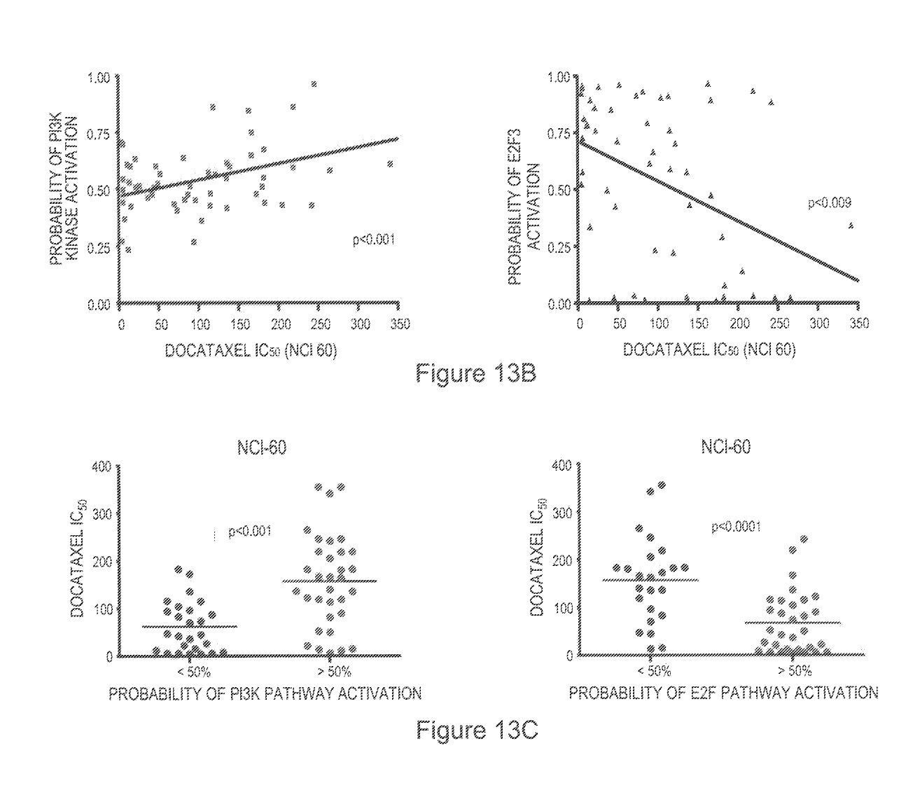

Most patients who are resistant to chemotherapeutic agents are then recruited into a second or third line therapy or enrolled in a clinical trial.^{38,59} Moreover, even those patients who initially respond to a given agent are likely to eventually suffer a relapse and in either case, additional therapeutic options are needed. As one approach to identifying such options, we have taken advantage of our recent work that describes the development of gene expression signatures that reflect the activation of several oncogenic pathways.³⁶ To illustrate the approach, we first stratified the NCI cell lines based on predicted docetaxel response and then examined the patterns of pathway deregulation associated with docetaxel sensitivity or resistance (FIG. 13A). Regression analysis revealed a significant relationship between PI3 kinase pathway deregulation and docetaxel resistance, as seen by the linear relationship (p=0.001) between the probability of PI3 kinase activation and the IC50 of docetaxel in the cell lines (FIG. 12 and Table 8).

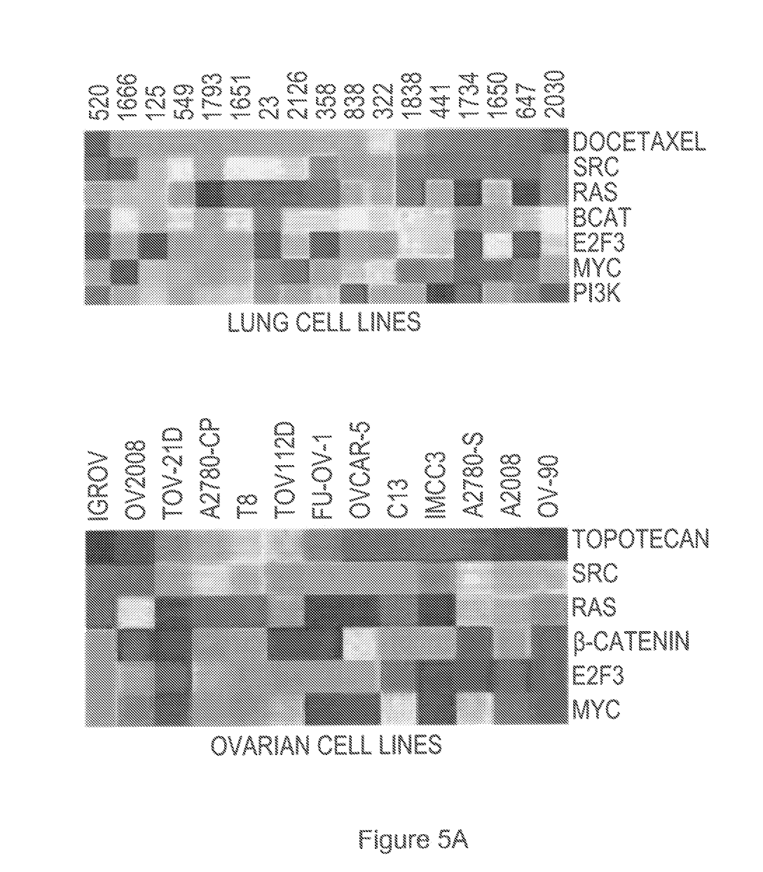

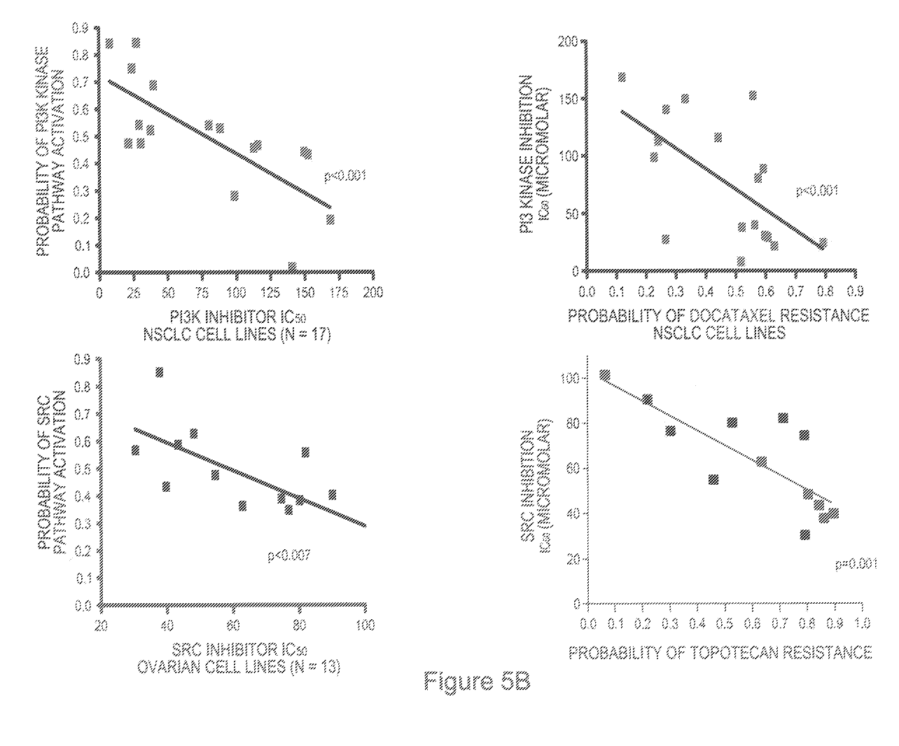

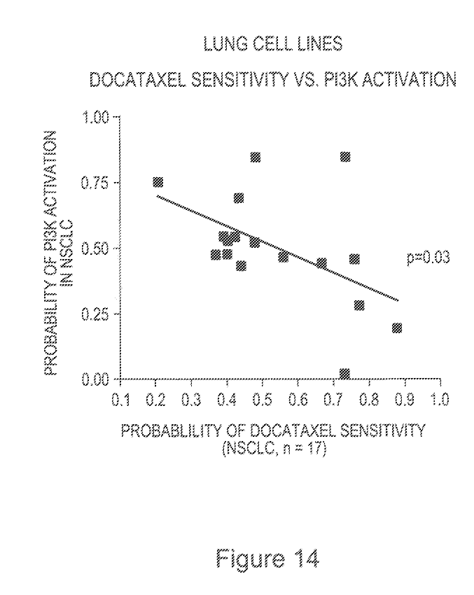

The results linking docetaxel resistance with deregulation of the PI3 kinase pathway, suggests an opportunity to employ a PI3 kinase inhibitor in this subgroup, given our recent observations that have demonstrated a linear positive correlation between the probability of pathway deregulation and targeted drug sensitivity.³⁶ To address this directly, we predicted docetaxel sensitivity and probability of oncogenic pathway deregulation using DNA microarray data from 17 NSCLC cell lines (FIG. 5A, top panel). Consistent with the analysis of the NCI-60 cell line panel, the cell lines predicted to be resistant to docetaxel were also predicted to exhibit PI3 kinase pathway activation (p=0.03, log-rank test, FIG. 14). In parallel, the lung cancer cell lines were subjected to assays for sensitivity to a PI3 kinase specific inhibitor (LY-294002), using a standard measure of cell proliferation.^{36, 38, 59} As shown by the analysis in FIG. 5B (top left panel), the cell lines showing an increased probability of PI3 kinase pathway activation were also more likely to respond to a PI3 kinase inhibitor (LY-294002) (p=0.001, log-rank test)). The same relationship held for prediction of resistance to docetaxel—these cells were more likely to be sensitive to PI3 kinase inhibition (p<0.001, log-rank test) (FIG. 5B, top right panel).

An analysis of a panel of ovarian cancer cell lines provided a second example. Ovarian cell lines that are predicted to be topotecan resistant (FIG. 5A, bottom panel) have a higher likelihood of Src pathway deregulation and there is a significant linear relationship (p=0.001, log rank) between the probability of topotecan resistance and sensitivity to a drug that inhibits the Src pathway (SU6656) (FIG. 5B, bottom right panel). The results of these assays clearly demonstrate an opportunity to potentially mitigate drug resistance (e.g., docetaxel or topotecan) using a specific pathway-targeted agent, based on a predictor developed from pathway deregulation (i.e., PI3 kinase or Src inhibition).

Taken together, these data demonstrate an approach to the identification of therapeutic options for chemotherapy resistant patients, as well as the identification of novel combinations for chemotherapy sensitive patients, and thus represents a potential strategy to a more effective treatment plan for cancer patients, after future prospective validations trials (FIG. 6).

### Example 7

### Methods

NCI-60 data. The (−log 10(M)) GI50/IC50, TGI (Total Growth Inhibition dose) and LC50 (50% cytotoxic dose) data was used to populate a matrix with MA TLAB software, with the relevant expression data for the individual cell lines. Where multiple entries for a drug screen existed (by NCS number), the entry with the largest number of replicates was included. Incomplete data were assigned as Nan (not a number) for statistical purposes. To develop an in vitro gene expression based predictor of sensitivity/resistance from the pharmacologic data used in the NCI-60 drug screen studies, we chose cell lines within the NCI-60 panel that would represent the extremes of sensitivity to a given chemotherapeutic agent (mean GI50+/−1 SD). Relevant expression data (updated data available on the Affymetrix U95A2 GeneChip) for the solid tumor cell lines and the respective pharmacological data for the chemotherapeutics was downloaded from the NCI website (http://dtp.nci.nih.gov/docs/cancer/cancer_data.html). The individual drug sensitivity and resistance data from the selected solid tumor NCI-60 cell lines was then used in a supervised analysis using binary regression methodologies, as described previously,⁶⁰ to develop models predictive of chemotherapeutic response.

Human ovarian cancer samples. We measured expression of 22,283 genes in 13 ovarian cancer cell lines and 119 advanced (FIGO stage III/IV) serous epithelial ovarian carcinomas using Affymetrix U133A GeneChips. All ovarian cancers were obtained at initial cytoreductive surgery from patients. All tissues were collected under the auspices of respective institutional (Duke University Medical Center and H. Lee Moffitt Cancer Center) IRB approved protocols involving written informed consent.

Full details of the methods used for RNA extraction and development of gene expression signatures representing deregulation of oncogenic pathways in the tumor samples were recently described.³⁶ Response to therapy was evaluated using standard criteria for patients with measurable disease, based upon WHO guidelines.²⁸

Lung and ovarian cancer cell culture. Total RNA was extracted and oncogenic pathway predictions was performed similar to the methods described previously.³⁶

Cross platform Affymetrix Gene Chip comparison. To map the probe sets across various generations of Affymetrix GeneChip arrays, we utilized an in-house program, Chip Comparer (http://tenero.duhs.duke.edu/genearray/perl/chip/chipcomparer.pl) as described previously.³⁶

Cell proliferation assays. Growth curves for cells were produced by plating 500-10,000 cells per well in 96-well plates. The growth of cells at 12 hr time points (from t=12 hrs) was determined using the CellTiter 96 Aqueous One 23 Solution Cell Proliferation Assay Kit by Promega, which is a colorimetric method for determining the number of growing cells.³⁶ The growth curves plot the growth rate of cells vs. each concentration of drug tested against individual cell lines. Cumulatively, these experiments determined the concentration of cells to use for each cell line, as well as the dosing range of the inhibitors. The final dose-response curves in our experiments plot the percent of cell population responding to the chemotherapy vs. the concentration of the drug for each cell line. Sensitivity to docetaxel and a phosphatidylinositol 3-kinase (PI3 kinase) inhibitor (LY-294002)³⁶ in 17 lung cell lines, and topotecan and a Src inhibitor (SU6656) in 13 ovarian cell lines was determined by quantifying the percent reduction in growth (versus DMSO controls) at 96 hrs using a standard MTT colorimetric assay.³⁶ Concentrations used ranged from 1-10 nM for docetaxel, 300 nM-10 μ/M (SU6656), and 300 nM-10M for LY-294002. All experiments were repeated at least three times.

Statistical analysis methods. Analysis of expression data are as previously described.^{36,60-62} Briefly, prior to statistical modeling, gene expression data is filtered to exclude probe sets with signals present at background noise levels, and for probe sets that do not vary significantly across samples. Each signature summarizes its constituent genes as a single expression profile, and is here derived as the top principal components of that set of genes. When predicting the chemosensitivity patterns or pathway activation of cancer cell lines or tumor samples, gene selection and identification is based on the training data, and then metagene values are computed using the principal components of the training data and additional cell line or tumor expression data. Bayesian fitting of binary probit regression models to the training data then permits an assessment of the relevance of the metagene signatures in within-sample classification,⁶⁰ and estimation and uncertainty assessments for the binary regression weights mapping metagenes to probabilities. To guard against over-fitting given the disproportionate number of variables to samples, we also performed leave-one-out cross validation analysis to test the stability and predictive capability of our model. Each sample was left out of the data set one at a time, the model was refitted (both the metagene factors and the partitions used) using the remaining samples, and the phenotype of the held out case was predicted and the certainty of the classification was calculated. Given a training set of expression vectors (of values across metagenes) representing two biological states, a binary probit regression model, of predictive probabilities for each of the two states (resistant vs. sensitive) for each case is estimated using Bayesian methods. Predictions of the relative oncogenic pathway status and chemosensitivity of the validation cell lines or tumor samples are then evaluated using methods previously described^{36,60} producing estimated relative probabilities—and associated measures of uncertainty—of chemosensitivity/oncogenic pathway deregulation across the validation samples. In instances where a combined probability of sensitivity to a combination chemotherapeutic regimen was required based on the individual drug sensitivity patterns, we employed the theorem for combined probabilities as described by Feller: [Probability (Pr) of (A), (B), (C) . . . (N)]=ΣPr (A)+Pr (B)+Pr (C) . . . [Pr (N)−[Pr(A)×Pr(B)×Pr(C) . . . ×Pr (N)]. Hierarchical clustering of tumor predictions was performed using Gene Cluster 3.0.⁶³ Genes and tumors were clustered using average linkage with the uncentered correlation similarity metric. Standard linear regression analyses and their significance (log rank test) were generated for the drug response data and correlation between drug response and probability of chemosensitivity/pathway deregulation using GraphPad® software.

### REFERENCE BIBLIOGRAPHY

1. Levin L, Simon R, Hryniuk W: Importance of multi agent chemotherapy regimens in ovarian carcinoma: dose intensity analysis. J Natl. Canc. Inst. 85: 1732-1742, 1993
2. McGuire W P, Hoskins W J, Brady M F, et al: Assessment of dose-intensive therapy in suboptimally debulked ovarian cancer: a Gynecologic Oncology Group study. J Clin. Oncol. 13:1589-1599, 1995
3. Jodrell Dl, Egorin M J, Canetta R M, et al: Relationships between carboplatin explosure and tumor response and toxicity in patients with ovarian cancer. J. Clin. Oncol. 10:520-528, 1992
4. McGuire W P, Hoskins W J, Brady M F, et al: Cyclophosphamide and cisplatin compared with paclitaxel and cisplatin in patients with stage III and stage IV ovarian cancer. N. Engl. J. Med. 334:1-6, 1996
5. McGuire W P, Brady M F, Ozols R F: The Gynecologic Oncology Group experience in ovarian cancer. Ann. Oncol. 10:29-34, 1999
6. Piccart M J, Bertelsen K, Stuart G, et al: Long-term follow-up confirms a survival advantage of the paclitaxel-cisplatin regimen over the cyclophosphamide-cisplatin combination in advanced ovarian cancer. Int. J. Gyneeol. Cancer 13:144-148, 2003
7. Wenham R M, Lancaster J M, Berchuck A: Molecular aspects of ovarian cancer. Best Pract. Res. Clin. Obstet. Gynaecol. 16:483-497, 2002
8. Berchuck A, Kohler M F, Marks J R, et al: The p53 tumor suppressor gene frequently is altered in gynecologic cancers. Am. J. Obstet. Gynecol. 170:246-252, 1994
9. Kohler M F, Marks J R, Wiseman R W, et al: Spectrum of mutation and frequency of allelic deletion of the p53 gene in ovarian cancer. J. Natl. Cane. Inst. 85:1513-1519, 1993
10. Havrilesky L, Alvarez A A, Whitaker R S, et al: Loss of expression of the p16 tumor suppressor gene is more frequent in advanced ovarian cancers lacking p53 mutations. Gynecol. Oncol. 83:491-500, 2001
11. Reles A, Wen W H, Schmider A, et al: Correlation of 53 mutations with resistance to platinum-based chemotherapy and shortened survival in ovarian cancer. Clinical Cancer Research 7:2984-2997, 2001
12. Schmider A, Gee C, Friedmann W, et al: p21 (WAF lICIP 1) protein expression is associated with prolonged survival but not with p53 expression in epithelial ovarian carcinoma. Gynecol. On col. 77:237-242, 2000
13. Wong K K, Cheng R S, Mok S C: Identification of differentially expressed genes from ovarian cancer cells by MICRO MAX cDNA microarray system. Biotechniques 30:670675, 2001
14. Welsh J B, Zarrinkar P P, Sapinoso L M, et al: Analysis of gene expression profiles in normal and neoplastic ovarian tissue samples identifies candidate molecular markers of epithelial ovarian cancer. Proc. Natl. Acad. Sci. USA 98: 1176-1181, 2001
15. Shridhar Y, Lee J-S, Pandita A, et al: Genetic analysis of early-versus late-state ovarian tumors. Cancer Res. 61:5895-5904, 2001
16. Schummer M, Ng W W, Bumgarner R E, et al: Comparative hybridization of an array of 21,500 ovarian cDNAs for the discovery of genes overexpressed in ovarian carcinomas. Gene 238:375-385, 1999
17. Ono K, Tanaka T, Tsunoda T, et al: Identification by cDNA micro array of genes involved in ovarian carcinogenesis. Cancer Res. 60:5007-5011, 2000
18. Sawiris G P, Sherman-Baust C A, Becker K G, et al: Development of a highly specialized cDNA array for the study and diagnosis of epithelial ovarian cancer. Cancer Res. 62:2923-2928, 2002
19. Jazaeri A A, Yee C J, Sotiriou C, et al: Gene expression profiles of BRCA1-linked, BRCA2-linked, and sporadic ovarian cancers. J Natl. Canc. Inst. 94:990-1000, 2002
20. Schaner M E, Ross D T, Ciaravino G, et al: Gene expression patterns in ovarian carcinomas. Mol. Biol. Cell 14:4376-4386, 2003
21. Lancaster J M, Dressman H, Whitaker R S, et al: Gene expression patterns that characterize advanced stage serous ovarian cancers. J Surgical Gynecol. Invest. 11:51-59, 2004
22. Berchuck A, Iversen E S, Lancaster J M, et al: Patterns of gene expression that characterize long term survival in advanced serous ovarian cancers. Clin. Can. Res. 11:3686-3696, 2005
23. Berchuck A, Iversen E, Lancaster J M, et al: Prediction of optimal versus suboptimal cytoreduction of advanced stage serous ovarian cancer using microarrays. Am. J. Obstet. Gynecol. 190:910-925, 2004
24. Jazaeri A A, Awtrey Cs, Chandramouli G V, et al: Gene expression profiles associated with response to chemotherapy in epithelial ovarian cancers. Clin. Cancer Res. 11:6300-6310, 2005
25. Helleman J, Jansen M P, Span P N, et al: Molecular profiling of platinum resistant ovarian cancer. Int. J. Cancer 118: 1963-1971, 2005
26. Spentzos D, Levine D A, Kolia s, et al: Unique gene expression profile based on pathologic response in epithelial ovarian cancer. J. Clin. Oncol. 23:7911-7918, 2005
27. Spentzos D, Levine D A, Ramoni M F, et al: Gene expression signature with independent prognostic significance in epithelial ovarian cancer. J. Clin. Oneal. 22:4700-4710, 2004
28. Miller A B, Hoogstraten B, Staquet M, et al: Reporting results of cancer treatment. Cancer 47:207-214, 1981
29. Rustin G J, Nelstrop A E, Bentzen S M, et al: Use of tumor markers in monitoring the course of ovarian cancer. Ann. Oncol. 10:21-27, 1999
30. Rustin G J, Nelstrop A E, McClean P, et al: Defining response of ovarian carcinoma to initial chemotherapy according to serum CA 125. J. Clin. Oncol. 14:1545-1551, 1996
31. Irizarry R A, Hobbs B, Collin F, et al: Exploration, normalization, and summaries of high density oligonucleotide array probe level data. Biostatistics 4:249-263, 2003
32. Bolstad B M, Irizarry R A, Astrand M, et al: A comparison of normalization methods for high density oligonucleotide array data based on variance and bias. Bioinformatics 19: 185-193, 2003
33. Lucus J, Carvalho C, Wang Q, et al: Sparse statistical modeling in gene expression genomics. Cambridge, Cambridge University Press, 2006
34. Rich J, Jones B, Hans C, et al: Gene expression profiling and genetic markers in glioblastoma survival. Cancer Res. 65:4051-4058, 2005
35. Hans C, Dobra A, West M: Shotgun stochastic search for regression with many candidate predictors. JASA in press. 2006
36. Bild A, Yao G, Chang J T, et al: Oncogenic pathway signatures in human cancers as a guide to targeted therapies. Nature 439:353-357, 2006.
37. Gyorrfy B, Surowiak P, Kiesslich O, Denkert C, Schafer R, Dietel M, Lage H: Gene expression profiling of 30 cancer cell lines predicts resistance towards 11 anticancer drugs at clinically achieved concentrations. Int. J Cancer 118(7): 1699-712, 2006
38. Minna, J D, Gazdar, A F, Sprang, S R & Herz, J: Cancer. A bull's eye for targeted lung cancer therapy. Science 304: 1458-1461, 2004
39. Jemal et al., CA Cancer J Clin., 53, 5-26, 2003
40. Cancer Facts and Figures: American Cancer Society, Atlanta, p. 11, 2002
41. Travis et al., Lung Cancer Principles and Practice, Lippincott-Raven, New York, pps. 361-395, 1996
42. Gazdar et al., Anticancer Res. 14:261-267
43. Niklinska et al., Folia Histochem. Cytobiol. 39:147-148, 2001
44. Parker et al, CA Cancer J Clin. 47:5-27, 1997
45. Chu et al, J Nat. Cancer Inst. 88:1571-1579, 1996
46. Baker, V V: Salvage therapy for recurrent epithelial ovarian cancer. Hematol. 011COI Clin. N. Am, 17: 977-988, 2003
47. Hansen, H H, Eisenhauer, E A, Hasen M, Neijt J P, Piccart M J, Sessa C, Thigpen J T: New cytostatis drugs in ovarian cancer. Ann. Oncol. 4:S63-S70, 1993
48. Herrin, V E, Thigpen J T: Chemotherapy for ovarian cancer: current concepts. Semin. Surg. On col. 17: 181-188, 1999
49. Staunton, J. E. et al. Chemosensitivity prediction by transcriptional profiling. Proc Natl Acad Sci USA 98: 10787-19792, 2001
50. Chang, J. c. et al. Gene expression profiling for the prediction of therapeutic response to docetaxel in patients with breast cancer. Lancet 362:362-369, 2003
51. Emi, M., Kim, R., Tanabe, K., Uchida, Y. & toge, T. Targeted therapy against Bcl-2-related proteins in breast cancer cells. Breast Cancer Res 7: R940-R952, 2005
52. Takahashi, T. et al. Cyclin A-associated kinase activity is needed for paclitaxel sensitivity. Mol Cancer Ther 4: 1039-1046, 2005
53. Modi, S. et al. Phosphorylated/activated HER2 as a marker of clinical resistance to single agent taxane chemotherapy for metastatic breast cancer. Cancer Invest 23: 483-487, 2005
54. Langer, R. et al. Association of pre therapeutic expression of chemotherapy-related genes with response to neoadjuvant chemotherapy in Barrett carcinoma. Clin Cancer Res. 11: 7462-7469, 2005
55. Rouzier, R. et al. Breast cancer molecular subtypes respond differently to preoperative chemotherapy. Clin Cancer Res. 11: 5678-5685, 2005
56. Rouzier, R. et al. Microbubule-associated protein tau: a marker of paclitaxel sensitivity on breast cancer. Proc Natl Acad Sci USA 102: 8315-8320, 2005
57. DeVita, V. T., Hellman, S. & Rosenberg, S. A. Cancer: Principles and Practice of Oncology, Lippincott-Raven, Philadelphia, 2005
58. Herbst, R. S. et at Clinical Cancer Advances 2005; Major research advances in cancer treatment, prevention, and screening—a report from the American Society of Clinical Oncology. J. Clin. Oncol. 24: 190-205, 2006
59. Broxterman, H. J. & Georgopapadakou, N. H. Anticancer therapeutics: Addictive targets, multi-targeted drugs, new drug combinations. Drug Resist Update 8: 183-197, 2005
60. Pittman, J. Huang, E., Wang, Q., Nevins, J R. & West, M. Bayesian analysis of binary prediction tree models for retrospectively sampled outcomes. Biostatistics 5: 587-601, 2004
61. West, M. et at Predicting the clinical status of human breast cancer by using gene expression profiles. Proc Natl Acad Sci USA 98: 11462-11467, 2001
62. Ihaka, R. & Gentleman, R. A language for data analysis and graphics. J. Comput. Graph. Stat. 5: 299-314, 1996
63. Eisen, M. B., Spellman, P. T., Brown, P. O. & Botstein, D. Cluster analysis and display of genome-wide expression patterns. Proc Natl Acad Sci USA 95:14863-14868, 1998

TABLE 1
5-Flourouracil responsivity predictor set

| 5FUProbe | | | | Entrez |
|---|---|---|---|---|
| Set ID web | Weight | Gene Symbol | Go biological process term | Gene ID |
| 151_s_at | −3.83685 | LOC92755 /// | fructose metabolic process /// | 203068 /// |
| | | TUBB | glycolysis /// cell motility /// | 92755 |
| | | | microtubule-based process /// | |
| | | | microtubule-based movement /// | |
| | | | metabolic process /// natural killer | |
| | | | cell mediated cytotoxicity /// protein | |
| | | | polymerization | |
| 1713_s_at | 4.712802 | CDKN2A | cell cycle checkpoint /// G1/S | 1029 |
| | | | transition of mitotic cell cycle /// | |
| | | | negative regulation of cell-matrix | |
| | | | adhesion /// DNA fragmentation | |
| | | | during apoptosis /// transcription /// | |
| | | | regulation of transcription, DNA- | |
| | | | dependent /// rRNA processing /// | |
| | | | negative regulation of protein kinase | |
| | | | activity /// apoptosis /// induction of | |
| | | | apoptosis /// induction of apoptosis | |
| | | | /// caspase activation /// cell cycle /// | |
| | | | cell cycle arrest /// negative | |
| | | | regulation of cell proliferation /// | |
| | | | apoptotic mitochondrial changes /// | |
| | | | senescence /// regulation of G2/M | |
| | | | transition of mitotic cell cycle /// | |
| | | | negative regulation of cell growth /// | |
| | | | negative regulation of B cell | |
| | | | proliferation /// regulation of protein | |
| | | | stability /// negative regulation of NF- | |
| | | | kappaB transcription factor activity | |
| | | | /// negative regulation of immature T | |
| | | | cell proliferation in the thymus /// | |
| | | | negative regulation of | |
| | | | phosphorylation /// negative | |
| | | | regulation of cyclin-dependent | |
| | | | protein kinase activity /// negative | |
| | | | regulation of cell cycle /// somatic | |
| | | | stem cell division /// negative | |
| | | | regulation of ubiquitin-protein ligase | |
| | | | activity | |
| 1882_g_at | 0.861954 | — | — | — |
| 31322_at | 1.401 | TRA@ | immune response /// cellular | 6955 |
| | | | defense response | |
| 31726_at | | GABRA3 | transport /// transport /// ion transport | 2556 |
| | | | /// chloride transport /// gamma- | |
| | | | aminobutyric acid signaling pathway | |
| | | | /// gamma-aminobutyric acid | |
| | | | signaling pathway | |
| 32308_r_at | −1.10479 | COL1A2 | skeletal development /// phosphate | 1278 |
| | | | transport /// transmembrane | |
| | | | receptor protein tyrosine kinase | |
| | | | signaling pathway /// sensory | |
| | | | perception of sound | |
| 32318_s_at | −1.23171 | ACTB | transport /// amino acid transport /// | 60 |
| | | | cell motility /// sensory perception of | |
| | | | sound /// arginine transport /// lysine | |
| | | | transport /// response to calcium ion | |
| 32610_at | 2.301947 | PDLIM4 | — | 8572 |
| 32755_at | 0.912152 | ACTA2 | — | 59 |
| 33437_at | −0.66656 | FTSJ1 | rRNA processing | 24140 |
| 33444_at | | NBR1 /// | — | 100133166 |
| | | LOC100133166 | | /// 4077 |
| 33659_at | 0.622566 | CFL1 | anti-apoptosis /// Rho protein signal | 1072 |
| | | | transduction /// actin cytoskeleton | |
| | | | organization and biogenesis | |
| 34377_at | 1.171995 | ATP1A2 | transport /// ion transport /// cation | 477 |
| | | | transport /// potassium ion transport | |
| | | | /// potassium ion transport /// sodium | |
| | | | ion transport /// sodium ion transport | |
| | | | /// regulation of striated muscle | |
| | | | contraction /// metabolic process /// | |
| | | | monovalent inorganic cation | |
| | | | transport /// proton transport /// | |
| | | | sperm motility /// regulation of | |
| | | | cellular pH | |
| 34454_r_at | −0.8324 | APOC2 /// | lipid metabolic process /// lipid | 344 /// 346 |
| | | APOC4 | metabolic process /// triacylglycerol | |
| | | | metabolic process /// phospholipid | |
| | | | metabolic process /// transport /// | |
| | | | lipid transport /// lipid catabolic | |
| | | | process /// cholesterol efflux /// | |
| | | | phospholipid efflux /// positive | |
| | | | regulation of lipoprotein lipase | |
| | | | activity | |
| 34545_at | | CCDC88C | regulation of protein amino acid | 440193 |
| | | | phosphorylation /// Wnt receptor | |
| | | | signaling pathway /// protein | |
| | | | destabilization /// protein | |
| | | | homooligomerization | |
| 34843_at | −2.25281 | — | — | — |
| 34905_at | 1.045288 | GRIK5 | transport /// ion transport /// synaptic | 2901 |
| | | | transmission | |
| 34954_r_at | 1.084054 | PDE5A | signal transduction /// signal | 8654 |
| | | | transduction /// cyclic nucleotide | |
| | | | metabolic process | |
| 35056_at | −2.52505 | ARSF | metabolic process | 416 |
| 35144_at | 0.689025 | ZC3H7B | — | 23264 |
| 35213_at | 0.693573 | WBP4 | mRNA processing /// RNA splicing | 11193 |
| 35816_at | −1.29531 | CSTB | adult locomotory behavior | 1476 |
| 35929_s_at | 1.027644 | TSPY1 /// | nucleosome assembly /// | 64591 /// |
| | | TSPY2 /// | multicellular organismal | 7258 /// |
| | | LOC728137 /// | development /// spermatogenesis /// | 728137 /// |
| | | LOC728395 /// | spermatogenesis /// gonadal | 728395 /// |
| | | LOC728412 | mesoderm development /// sex | 728412 |
| | | | differentiation /// cell proliferation /// | |
| | | | cell differentiation | |
| 36245_at | −1.6361 | HTR2B | signal transduction /// G-protein | 3357 |
| | | | coupled receptor protein signaling | |
| | | | pathway /// G-protein signaling, | |
| | | | coupled to IP3 second messenger | |
| | | | (phospholipase C activating) /// | |
| | | | heart development /// blood | |
| | | | circulation /// positive regulation of I- | |
| | | | kappaB kinase/NF-kappaB cascade | |
| 36453_at | 1.22492 | KBTBD11 | — | 9920 |
| 36549_at | −0.92503 | SLC25A17 | transport /// transport /// | 10478 |
| | | | mitochondrial transport | |
| 37349_r_at | 1.984623 | HMGN3 | — | 9324 |
| 37361_at | −1.79231 | FIBP | fibroblast growth factor receptor | 9158 |
| | | | signaling pathway | |
| 37437_at | −0.9449 | IFT140 | cell communication | 9742 |
| 37802_r_at | 2.334769 | FAM63B | — | 54629 |
| 37860_at | 1.40471 | ZNF337 | transcription /// regulation of | 26152 |
| | | | transcription, DNA-dependent | |
| 39783_at | 0.802025 | KIAA0100 | tricarboxylic acid cycle | 9703 |
| 39898_at | −0.9176 | FAM13C1 | — | 220965 |
| 40104_at | −0.70116 | STK25 | protein amino acid phosphorylation | 10494 |
| | | | /// response to oxidative stress /// | |
| | | | signal transduction | |
| 40452_at | −1.26932 | CPNE1 | lipid metabolic process /// vesicle- | 8904 |
| | | | mediated transport | |
| 40471_at | | PEX19 | protein targeting to peroxisome /// | 5824 |
| | | | peroxisome organization and | |
| | | | biogenesis /// peroxisome | |
| | | | organization and biogenesis | |
| 40536_f_at | | EIF5B | translation /// regulation of | 9669 |
| | | | translational initiation /// regulation of | |
| | | | translational initiation | |
| 40886_at | −1.81682 | EEF1A1 /// | angiogenesis /// translation /// | 100132804 |
| | | EEF1AL3 /// | translational elongation /// | /// 158078 |
| | | LOC100132804 | translational elongation /// | /// 1915 |
| | | | translational elongation /// lipid | |
| | | | transport /// multicellular organismal | |
| | | | development /// cell differentiation /// | |
| | | | lipoprotein metabolic process | |
| 40983_s_at | 1.828091 | SRR | amino acid metabolic process /// | 63826 |
| | | | amino acid metabolic process /// L- | |
| | | | serine metabolic process /// | |
| | | | metabolic process /// serine family | |
| | | | amino acid metabolic process | |
| 41058_g_at | 0.545125 | THEM2 | — | 55856 |
| 41536_at | 0.453047 | ID4 | regulation of transcription from RNA | 3400 |
| | | | polymerase II promoter /// | |
| | | | neuroblast proliferation /// positive | |
| | | | regulation of cell proliferation /// | |
| | | | negative regulation of transcription | |
| | | | /// regulation of transcription /// | |
| | | | negative regulation of neuron | |
| | | | differentiation /// negative regulation | |
| | | | of astrocyte differentiation | |
| 41868_at | −1.46067 | GGT1 /// | amino acid metabolic process /// | 2678 /// |
| | | GGTLC2 | glutathione biosynthetic process /// | 91227 |
| | | | glutathione biosynthetic process | |
| 427_f_at | −0.72026 | IFNA10 | defense response /// cell-cell | 3446 |
| | | | signaling /// response to virus /// | |
| | | | response to virus | |
| 429_f_at | 0.512718 | TUBB4 | microtubule-based process /// | 10382 |
| | | | microtubule-based movement /// | |
| | | | mitosis /// neuron differentiation /// | |
| | | | protein polymerization | |
| 471_f_at | −0.74815 | TUBB3 | microtubule-based process /// | 10381 |
| | | | microtubule-based movement /// | |
| | | | mitosis /// signal transduction /// G- | |
| | | | protein coupled receptor protein | |
| | | | signaling pathway /// G-protein | |
| | | | signaling, coupled to cyclic | |
| | | | nucleotide second messenger /// | |
| | | | multicellular organismal | |
| | | | development /// UV protection /// | |
| | | | neuron differentiation /// protein | |
| | | | polymerization | |

TABLE 2
Adriamycin responsivity predictor set

| Web site | | | | |
|---|---|---|---|---|
| Adria | | | | |
| Probe Set | | Gene | | Entrez |
| ID | Weight | Symbol | Go biological process term | Gene ID |
| 1051_g_at | −0.94348 | MLANA | — | 2315 |
| 110_at | 1.234027 | CSPG4 | angiogenesis /// cell motility /// signal | 1464 |
| | | | transduction /// multicellular organismal | |
| | | | development /// cell differentiation /// | |
| | | | tissue remodeling | |
| 1319_at | 0.677949 | DDR2 | protein amino acid phosphorylation /// cell | 4921 |
| | | | adhesion /// cell adhesion /// signal | |
| | | | transduction /// transmembrane receptor | |
| | | | protein tyrosine kinase signaling pathway | |
| | | | /// positive regulation of cell proliferation | |
| 1519_at | −1.85295 | ETS2 | skeletal development /// regulation of | 2114 |
| | | | transcription, DNA-dependent | |
| 1537_at | 2.591759 | EGFR | ossification /// protein amino acid | 1956 |
| | | | phosphorylation /// response to stress /// | |
| | | | cell cycle /// signal transduction /// cell | |
| | | | surface receptor linked signal | |
| | | | transduction /// transmembrane receptor | |
| | | | protein tyrosine kinase signaling pathway | |
| | | | /// epidermal growth factor receptor | |
| | | | signaling pathway /// epidermal growth | |
| | | | factor receptor signaling pathway /// | |
| | | | activation of phospholipase C activity /// | |
| | | | cell proliferation /// positive regulation of | |
| | | | cell proliferation /// cell-cell adhesion /// | |
| | | | positive regulation of cell migration /// | |
| | | | positive regulation of phosphorylation /// | |
| | | | calcium-dependent phospholipase A2 | |
| | | | activation /// positive regulation of MAP | |
| | | | kinase activity /// positive regulation of | |
| | | | nitric oxide biosynthetic process /// | |
| | | | negative regulation of cell cycle /// positive | |
| | | | regulation of epithelial cell proliferation /// | |
| | | | regulation of peptidyl-tyrosine | |
| | | | phosphorylation /// regulation of nitric- | |
| | | | oxide synthase activity /// protein insertion | |
| | | | into membrane | |
| 2011_s_at | −2.46428 | BIK | apoptosis /// induction of apoptosis /// | 638 |
| | | | induction of apoptosis /// apoptotic | |
| | | | program /// regulation of apoptosis | |
| 266_s_at | 1.920993 | CD24 | response to hypoxia /// cell activation /// | 934 |
| | | | regulation of cytokine and chemokine | |
| | | | mediated signaling pathway /// regulation | |
| | | | of cytokine and chemokine mediated | |
| | | | signaling pathway /// response to | |
| | | | molecule of bacterial origin /// response to | |
| | | | molecule of bacterial origin /// immune | |
| | | | response-regulating cell surface receptor | |
| | | | signaling pathway /// elevation of cytosolic | |
| | | | calcium ion concentration /// | |
| | | | neuromuscular synaptic transmission /// | |
| | | | induction of apoptosis by intracellular | |
| | | | signals /// Wnt receptor signaling pathway | |
| | | | /// cell-cell adhesion /// cell migration /// | |
| | | | cell migration /// regulation of epithelial | |
| | | | cell differentiation /// T cell costimulation | |
| | | | /// B cell receptor transport into membrane | |
| | | | raft /// chemokine receptor transport out of | |
| | | | membrane raft /// negative regulation of | |
| | | | transforming growth factor-beta3 | |
| | | | production /// positive regulation of | |
| | | | activated T cell proliferation /// regulation | |
| | | | of phosphorylation /// cholesterol | |
| | | | homeostasis /// cholesterol homeostasis | |
| | | | /// positive regulation of MAP kinase | |
| | | | activity /// regulation of MAPKKK cascade | |
| | | | /// response to estrogen stimulus /// | |
| | | | respiratory burst /// synaptic vesicle | |
| | | | endocytosis | |
| 32139_at | 2.120382 | ZNF185 | — | 7739 |
| 32168_s_at | −0.56607 | RCAN1 | signal transduction /// central nervous | 1827 |
| | | | system development /// blood circulation | |
| | | | /// calcium-mediated signaling | |
| 32612_at | −1.93636 | GSN | actin filament polymerization /// actin | 2934 |
| | | | filament polymerization /// actin filament | |
| | | | severing /// actin filament severing /// | |
| | | | barbed-end actin filament capping /// | |
| | | | barbed-end actin filament capping | |
| 32718_at | −1.07596 | TPST1 | peptidyl-tyrosine sulfation /// inflammatory | 8460 |
| | | | response | |
| 32821_at | 1.172906 | LCN2 | transport | 3934 |
| 32967_at | 2.725153 | FAIM3 | anti-apoptosis /// immune response /// | 9214 |
| | | | cellular defense response | |
| 33004_g_at | 1.165497 | NCK2 | regulation of translation /// signal | 8440 |
| | | | transduction /// signal complex assembly | |
| | | | /// epidermal growth factor receptor | |
| | | | signaling pathway /// regulation of | |
| | | | epidermal growth factor receptor activity | |
| | | | /// negative regulation of cell proliferation | |
| | | | /// positive regulation of actin filament | |
| | | | polymerization /// positive regulation of T | |
| | | | cell proliferation /// T cell activation | |
| 33240_at | −2.07044 | PDZRN3 | — | 23024 |
| 33409_at | −0.84774 | FKBP2 | protein folding | 2286 |
| 33824_at | 0.8914 | KRT8 | cytoskeleton organization and biogenesis | 3856 |
| | | | /// response to other organism | |
| 33853_s_at | 2.187597 | NRP2 | angiogenesis /// cell adhesion /// cell | 8828 |
| | | | adhesion /// multicellular organismal | |
| | | | development /// nervous system | |
| | | | development /// axon guidance /// cell | |
| | | | differentiation /// cell redox homeostasis | |
| 33892_at | −1.14625 | PKP2 | cell adhesion /// cell-cell adhesion | 5318 |
| 33904_at | −1.29549 | CLDN3 | response to hypoxia /// calcium- | 1365 |
| | | | independent cell-cell adhesion /// calcium- | |
| | | | independent cell-cell adhesion | |
| 33908_at | −1.35671 | CAPN1 | proteolysis /// positive regulation of cell | 823 |
| | | | proliferation | |
| 33942_s_at | −1.20596 | STXBP1 | transport /// vesicle docking during | 6812 |
| | | | exocytosis /// protein transport /// vesicle- | |
| | | | mediated transport | |
| 33956_at | 1.164645 | LY96 | inflammatory response /// immune | 23643 |
| | | | response /// cellular defense response /// | |
| | | | cell surface receptor linked signal | |
| | | | transduction | |
| 34213_at | −1.32674 | WWC1 | — | 23286 |
| 34303_at | 1.15829 | C10orf56 | — | 219654 |
| 34348_at | −0.97728 | SPINT2 /// | cell motility | 100130414 |
| | | LOC100130414 | | /// |
| | | | | 10653 |
| 34859_at | 1.376672 | MAGED2 | translation | 10916 |
| 34885_at | 1.474456 | SYNGR2 | — | 9144 |
| 34993_at | 3.241691 | SGCD | cytoskeleton organization and biogenesis | 6444 |
| | | | /// muscle development | |
| 35280_at | −0.95845 | LAMC2 | cell adhesion /// epidermis development | 3918 |
| 35444_at | −1.24187 | C19orf21 | — | 126353 |
| 35681_r_at | 2.082145 | ZEB2 | transcription /// regulation of transcription, | 9839 |
| | | | DNA-dependent /// nervous system | |
| | | | development /// negative regulation of | |
| | | | transcription /// regulation of transcription | |
| 35766_at | 1.257264 | KRT18 | cell cycle /// anatomical structure | 3875 |
| | | | morphogenesis /// Golgi to plasma | |
| | | | membrane CFTR protein transport /// | |
| | | | negative regulation of apoptosis | |
| 35807_at | −1.93358 | CYBA | superoxide metabolic process /// transport | 1535 |
| | | | /// oxidation reduction | |
| 36133_at | −0.92039 | DSP | epidermis development /// peptide cross- | 1832 |
| | | | linking /// keratinocyte differentiation | |
| 36618_g_at | 0.683166 | ID1 | regulation of transcription from RNA | 3397 |
| | | | polymerase II promoter /// multicellular | |
| | | | organismal development /// negative | |
| | | | regulation of transcription /// negative | |
| | | | regulation of transcription factor activity /// | |
| | | | regulation of transcription | |
| 36619_r_at | 2.621706 | ID1 | regulation of transcription from RNA | 3397 |
| | | | polymerase II promoter /// multicellular | |
| | | | organismal development /// negative | |
| | | | regulation of transcription /// negative | |
| | | | regulation of transcription factor activity /// | |
| | | | regulation of transcription | |
| 36795_at | −0.62444 | PSAP | lipid metabolic process /// sphingolipid | 5660 |
| | | | metabolic process /// glycosphingolipid | |
| | | | metabolic process /// lipid transport /// | |
| | | | lysosome organization and biogenesis | |
| 36828_at | −0.58068 | ZNF629 | transcription /// regulation of transcription, | 23361 |
| | | | DNA-dependent | |
| 36849_at | 0.612692 | ARHGAP29 | signal transduction /// intracellular | 9411 |
| | | | signaling cascade /// Rho protein signal | |
| | | | transduction | |
| 37117_at | −0.53853 | ARHGAP8 /// | cell cycle /// signal transduction /// actin | 23779 /// |
| | | PRR5 /// | cytoskeleton organization and biogenesis | 553158 |
| | | LOC553158 | /// positive regulation of cell migration /// | /// 55615 |
| | | | negative regulation of cell cycle | |
| 37251_s_at | | GPM6B | multicellular organismal development /// | 2824 |
| | | | nervous system development /// nervous | |
| | | | system development /// cell differentiation | |
| 37327_at | 1.236668 | EGFR | ossification /// protein amino acid | 1956 |
| | | | phosphorylation /// response to stress /// | |
| | | | cell cycle /// signal transduction /// cell | |
| | | | surface receptor linked signal | |
| | | | transduction /// transmembrane receptor | |
| | | | protein tyrosine kinase signaling pathway | |
| | | | /// epidermal growth factor receptor | |
| | | | signaling pathway /// epidermal growth | |
| | | | factor receptor signaling pathway /// | |
| | | | activation of phospholipase C activity /// | |
| | | | cell proliferation /// positive regulation of | |
| | | | cell proliferation /// cell-cell adhesion /// | |
| | | | positive regulation of cell migration /// | |
| | | | positive regulation of phosphorylation /// | |
| | | | calcium-dependent phospholipase A2 | |
| | | | activation /// positive regulation of MAP | |
| | | | kinase activity /// positive regulation of | |
| | | | nitric oxide biosynthetic process /// | |
| | | | negative regulation of cell cycle /// positive | |
| | | | regulation of epithelial cell proliferation /// | |
| | | | regulation of peptidyl-tyrosine | |
| | | | phosphorylation /// regulation of nitric- | |
| | | | oxide synthase activity /// protein insertion | |
| | | | into membrane | |
| 37345_at | −1.49834 | CALU | — | 813 |
| 37552_at | 1.600714 | KCNK1 | transport /// ion transport /// potassium ion | 3775 |
| | | | transport /// potassium ion transport | |
| 37695_at | 1.263311 | RNF144A | ubiquitin cycle | 9781 |
| 37743_at | −2.36633 | FEZ1 | cell adhesion /// nervous system | 9638 |
| | | | development /// axon guidance | |
| 37749_at | 0.830285 | MEST | mesoderm development | 4232 |
| 37926_at | 1.591841 | KLF5 | angiogenesis /// transcription /// regulation | 688 |
| | | | of transcription, DNA-dependent /// | |
| | | | transcription from RNA polymerase II | |
| | | | promoter /// positive regulation of cell | |
| | | | proliferation /// microvillus biogenesis /// | |
| | | | positive regulation of transcription | |
| 38004_at | −0.6707 | CSPG4 | angiogenesis /// cell motility /// signal | 1464 |
| | | | transduction /// multicellular organismal | |
| | | | development /// cell differentiation /// | |
| | | | tissue remodeling | |
| 38078_at | −1.44495 | FLNB | cytoskeletal anchoring /// signal | 2317 |
| | | | transduction /// multicellular organismal | |
| | | | development /// skeletal muscle | |
| | | | development /// actin cytoskeleton | |
| | | | organization and biogenesis /// cell | |
| | | | differentiation | |
| 38119_at | −3.15147 | GYPC | protein amino acid N-linked glycosylation | 2995 |
| | | | /// protein amino acid O-linked | |
| | | | glycosylation /// organ morphogenesis | |
| 38122_at | 1.601555 | SLC23A2 | nucleobase, nucleoside, nucleotide and | 9962 |
| | | | nucleic acid metabolic process /// | |
| | | | transport /// ion transport /// sodium ion | |
| | | | transport /// nucleobase transport /// | |
| | | | molecular hydrogen transport /// L- | |
| | | | ascorbic acid metabolic process | |
| 38227_at | | MITF | transcription /// regulation of transcription, | 4286 |
| | | | DNA-dependent /// regulation of | |
| | | | transcription, DNA-dependent /// | |
| | | | multicellular organismal development /// | |
| | | | sensory perception of sound /// | |
| | | | melanocyte differentiation /// regulation of | |
| | | | transcription | |
| 38297_at | 0.897307 | PITPNM1 | lipid metabolic process /// transport /// | 9600 |
| | | | brain development /// phototransduction /// | |
| | | | protein transport | |
| 38379_at | −1.22485 | GPNMB | negative regulation of cell proliferation | 10457 |
| 38653_at | −1.26954 | PMP22 | synaptic transmission /// peripheral | 5376 |
| | | | nervous system development /// sensory | |
| | | | perception of sound /// mechanosensory | |
| | | | behavior /// negative regulation of cell | |
| | | | proliferation | |
| 39214_at | −2.83871 | PLXNB3 | protein amino acid phosphorylation /// | 5365 |
| | | | signal transduction /// multicellular | |
| | | | organismal development | |
| 39271_at | −2.40695 | MIA | cell-matrix adhesion /// cell proliferation /// | 8190 |
| | | | extracellular matrix organization and | |
| | | | biogenesis | |
| 39316_at | 1.090483 | RAB40C | ubiquitin cycle /// intracellular signaling | 57799 |
| | | | cascade /// small GTPase mediated signal | |
| | | | transduction /// protein transport | |
| 39386_at | −2.27665 | MAD2L1BP | regulation of exit from mitosis | 9587 |
| 39801_at | −1.2452 | PLOD3 | protein modification process /// protein | 8985 |
| | | | metabolic process | |
| 40103_at | 1.069164 | EZR | cytoskeletal anchoring /// regulation of cell | 7430 |
| | | | shape /// actin filament bundle formation | |
| 40202_at | 1.070163 | KLF9 | transcription /// regulation of transcription, | 687 |
| | | | DNA-dependent /// regulation of | |
| | | | transcription from RNA polymerase II | |
| | | | promoter /// embryo implantation /// | |
| | | | regulation of transcription /// progesterone | |
| | | | receptor signaling pathway | |
| 40434_at | 1.126169 | PODXL | negative regulation of cell adhesion /// | 5420 |
| | | | leukocyte migration | |
| 40568_at | −0.48906 | ATP6V1B2 | ATP biosynthetic process /// transport /// | 526 |
| | | | ion transport /// ATP synthesis coupled | |
| | | | proton transport /// energy coupled proton | |
| | | | transport, against electrochemical | |
| | | | gradient /// proton transport /// proton | |
| | | | transport | |
| 40926_at | −1.2209 | SLC6A8 | neurotransmitter uptake /// transport /// | 6535 |
| | | | transport /// ion transport /// sodium ion | |
| | | | transport /// neurotransmitter transport /// | |
| | | | muscle contraction | |
| 41158_at | 1.088079 | PLP1 | synaptic transmission /// axon | 5354 |
| | | | ensheathment | |
| 41294_at | −1.29363 | KRT7 | DNA replication /// regulation of | 3855 |
| | | | translation /// cytoskeleton organization | |
| | | | and biogenesis /// interphase | |
| 41359_at | 1.004171 | PKP3 | cell adhesion | 11187 |
| 41378_at | −2.53685 | — | — | — |
| 41453_at | 1.276055 | DLG3 | negative regulation of cell proliferation | 1741 |
| 41503_at | 0.635937 | ZHX2 | transcription /// regulation of transcription, | 22882 |
| | | | DNA-dependent /// mRNA catabolic | |
| | | | process /// regulation of transcription /// | |
| | | | negative regulation of transcription, DNA- | |
| | | | dependent | |
| 41610_at | −2.14951 | LAMA5 | angiogenesis /// cytoskeleton organization | 3911 |
| | | | and biogenesis /// cell adhesion /// | |
| | | | integrin-mediated signaling pathway /// | |
| | | | cell recognition /// cell proliferation /// | |
| | | | embryonic development /// cell migration | |
| | | | /// cell differentiation /// regulation of cell | |
| | | | adhesion /// regulation of cell migration /// | |
| | | | endothelial cell differentiation /// regulation | |
| | | | of embryonic development /// focal | |
| | | | adhesion formation | |
| 41644_at | −1.27145 | SASH1 | cell cycle /// negative regulation of cell | 23328 |
| | | | cycle | |
| 41839_at | −1.30948 | GAS1 | cell cycle /// cell cycle arrest /// cell cycle | 2619 |
| | | | arrest /// negative regulation of cell | |
| | | | proliferation /// programmed cell death /// | |
| | | | cell fate commitment /// negative | |
| | | | regulation of S phase of mitotic cell cycle | |
| | | | /// eye morphogenesis /// negative | |
| | | | regulation of epithelial cell proliferation | |
| 575_s_at | 0.929663 | TACSTD1 | — | 4072 |
| 661_at | 2.176958 | GAS1 | cell cycle /// cell cycle arrest /// cell cycle | 2619 |
| | | | arrest /// negative regulation of cell | |
| | | | proliferation /// programmed cell death /// | |
| | | | cell fate commitment /// negative | |
| | | | regulation of S phase of mitotic cell cycle | |
| | | | /// eye morphogenesis /// negative | |
| | | | regulation of epithelial cell proliferation | |
| 953_g_at | −0.89283 | — | — | — |
| 999_at | 1.075154 | CYP27A1 | oxidation reduction | 1593 |

TABLE 3
Cytotaxan responsivity predictor set

| Cytoxan | | | | |
|---|---|---|---|---|
| Probe Set | | | | Entrez |
| ID | Weight | Gene Symbol | Go biological process term | Gene ID |
| 1356_at | 4.874357 | DAP3 | apoptosis /// induction of apoptosis by | 7818 |
| | | | extracellular signals | |
| 31511_at | 3.788663 | RPS9 | translation /// translation | 6203 |
| 32252_at | 5.394493 | TTR | thyroid hormone generation /// transport /// | 7276 |
| | | | transport | |
| 32318_s_at | 3.101326 | ACTB | transport /// amino acid transport /// cell | 60 |
| | | | motility /// sensory perception of sound /// | |
| | | | arginine transport /// lysine transport /// | |
| | | | response to calcium ion | |
| 32434_at | −2.569863 | MARCKS | cell motility | 4082 |
| 32893_s_at | 1.254223 | GGT1 /// | amino acid metabolic process /// | 2678 /// |
| | | GGT3P /// | glutathione biosynthetic process /// | 2679 /// |
| | | GGTLC2 /// | glutathione biosynthetic process | 650860 /// |
| | | GGTLC1 /// | | 728226 /// |
| | | LOC650860 /// | | 728441 /// |
| | | GGTLC3 /// | | 91227 /// |
| | | GGT2 | | 92086 |
| 33145_at | −1.175404 | FANCA | DNA repair /// DNA repair /// protein | 2175 |
| | | | complex assembly /// response to DNA | |
| | | | damage stimulus | |
| 33362_at | 2.071209 | CDC42EP3 | signal transduction /// regulation of cell | 10602 |
| | | | shape | |
| 33919_at | 2.705049 | TSPAN4 | protein complex assembly | 7106 |
| 34246_at | −2.812789 | C6orf145 | cell communication | 221749 |
| 35352_at | 4.019153 | ARNT2 | response to hypoxia /// in utero embryonic | 9915 |
| | | | development /// in utero embryonic | |
| | | | development /// transcription /// regulation | |
| | | | of transcription, DNA-dependent /// signal | |
| | | | transduction /// central nervous system | |
| | | | development /// central nervous system | |
| | | | development /// regulation of transcription | |
| | | | /// regulation of transcription /// positive | |
| | | | regulation of transcription /// positive | |
| | | | regulation of transcription /// positive | |
| | | | regulation of transcription from RNA | |
| | | | polymerase II promoter | |
| 356_at | 1.879373 | KIF22 | microtubule-based movement /// mitosis | 3835 |
| 35763_at | −0.870241 | NBEAL2 | — | 23218 |
| 36119_at | 2.689896 | CAV1 | inactivation of MAPK activity /// | 857 |
| | | | vasculogenesis /// response to hypoxia /// | |
| | | | negative regulation of endothelial cell | |
| | | | proliferation /// triacylglycerol metabolic | |
| | | | process /// calcium ion transport /// cellular | |
| | | | calcium ion homeostasis /// endocytosis /// | |
| | | | regulation of smooth muscle contraction /// | |
| | | | skeletal muscle development /// protein | |
| | | | localization /// vesicle organization and | |
| | | | biogenesis /// regulation of fatty acid | |
| | | | metabolic process /// sequestering of lipid | |
| | | | /// regulation of blood coagulation /// | |
| | | | cholesterol transport /// negative regulation | |
| | | | of epithelial cell differentiation /// | |
| | | | mammary gland development /// nitric | |
| | | | oxide homeostasis /// cholesterol | |
| | | | homeostasis /// cholesterol homeostasis /// | |
| | | | negative regulation of MAPKKK cascade | |
| | | | /// negative regulation of nitric oxide | |
| | | | biosynthetic process /// positive regulation | |
| | | | of vasoconstriction /// negative regulation | |
| | | | of vasodilation /// negative regulation of | |
| | | | JAK-STAT cascade /// positive regulation | |
| | | | of metalloenzyme activity /// protein | |
| | | | homooligomerization /// membrane | |
| | | | depolarization /// regulation of peptidase | |
| | | | activity /// calcium ion homeostasis /// | |
| | | | mammary gland involution | |
| 36192_at | 1.514496 | SCRN1 | proteolysis /// exocytosis /// exocytosis | 9805 |
| 36536_at | 4.000312 | SCHIP1 | — | 29970 |
| 37375_at | 3.575144 | PHLDB1 | — | 23187 |
| 37680_at | −1.228293 | AKAP12 | protein targeting /// signal transduction /// | 9590 |
| | | | G-protein coupled receptor protein | |
| | | | signaling pathway | |
| 37745_s_at | 3.741699 | ST5 | — | 6764 |
| 38288_at | 4.706992 | SNAI2 | negative regulation of transcription from | 6591 |
| | | | RNA polymerase II promoter /// | |
| | | | transcription /// regulation of transcription, | |
| | | | DNA-dependent /// multicellular | |
| | | | organismal development /// ectoderm and | |
| | | | mesoderm interaction /// sensory | |
| | | | perception of sound /// response to | |
| | | | radiation | |
| 38375_at | 0.969629 | ESD | release of cytochrome c from mitochondria | 2098 |
| | | | /// apoptosis /// induction of apoptosis via | |
| | | | death domain receptors /// apoptotic | |
| | | | mitochondrial changes /// regulation of | |
| | | | apoptosis /// positive regulation of | |
| | | | apoptosis /// neuron apoptosis | |
| 38479_at | 1.720282 | ANP32B | — | 10541 |
| 39170_at | 3.438103 | CD59 | defense response /// immune response /// | 966 |
| | | | cell surface receptor linked signal | |
| | | | transduction /// blood coagulation | |
| 39329_at | 2.366823 | ACTN1 | regulation of apoptosis /// focal adhesion | 87 |
| | | | formation /// actin filament bundle | |
| | | | formation /// negative regulation of cell | |
| | | | motility | |
| 39351_at | −1.350962 | CD59 | defense response /// immune response /// | 966 |
| | | | cell surface receptor linked signal | |
| | | | transduction /// blood coagulation | |
| 39696_at | 5.888778 | PEG10 | proteolysis /// apoptosis /// cell | 23089 |
| | | | differentiation /// transposition | |
| 39750_at | −2.622822 | — | — | — |
| 40213_at | 3.382652 | SMARCA1 | chromatin remodeling /// transcription /// | 6594 |
| | | | regulation of transcription, DNA- | |
| | | | dependent /// brain development /// | |
| | | | chromatin modification /// neuron | |
| | | | differentiation /// ATP-dependent | |
| | | | chromatin remodeling /// positive | |
| | | | regulation of gene-specific transcription | |
| 40394_at | 3.379691 | GGCX | protein modification process /// blood | 2677 |
| | | | coagulation /// peptidyl-glutamic acid | |
| | | | carboxylation | |
| 40855_at | −0.689293 | SAMD4A | positive regulation of translation | 23034 |
| 40953_at | 2.35874 | CNN3 | smooth muscle contraction /// muscle | 1266 |
| | | | development /// actomyosin structure | |
| | | | organization and biogenesis | |
| 41195_at | −1.931524 | LPP | cell adhesion | 4026 |
| 41403_at | 1.485089 | SNRPF | mRNA processing /// RNA splicing /// RNA | 6636 |
| | | | splicing /// mRNA metabolic process | |
| 41449_at | 2.035738 | SGCE | cell-matrix adhesion /// muscle | 8910 |
| | | | development | |
| 41739_s_at | 1.395914 | CALD1 | cell motility /// muscle contraction | 800 |
| 41758_at | 2.920572 | TMEM184B | — | 25829 |

TABLE 4
Docetaxol responsivity predictor set

| Doce | | | | |
|---|---|---|---|---|
| Probe Set | | | | Entrez |
| ID | Weight | Gene Symbol | Go biological process term | Gene ID |
| 1003_s_at | −1.586523 | CXCR5 | cell motility /// signal transduction /// | 643 |
| | | | G-protein coupled receptor protein | |
| | | | signaling pathway /// G-protein | |
| | | | coupled receptor protein signaling | |
| | | | pathway /// B cell activation /// lymph | |
| | | | node development | |
| 1420_s_at | −1.306835 | EIF4A2 | translation /// regulation of | 1974 |
| | | | translational initiation | |
| 1567_at | −1.007947 | FLT1 | angiogenesis /// patterning of blood | 2321 |
| | | | vessels /// protein amino acid | |
| | | | phosphorylation /// transmembrane | |
| | | | receptor protein tyrosine kinase | |
| | | | signaling pathway /// | |
| | | | transmembrane receptor protein | |
| | | | tyrosine kinase signaling pathway /// | |
| | | | multicellular organismal | |
| | | | development /// female pregnancy /// | |
| | | | positive regulation of cell | |
| | | | proliferation /// cell migration /// cell | |
| | | | differentiation /// vascular endothelial | |
| | | | growth factor receptor signaling | |
| | | | pathway | |
| 1861_at | −1.210512 | BAD | apoptosis /// induction of apoptosis | 572 |
| | | | /// apoptotic program | |
| 32085_at | 1.984601 | PIP5K3 | intracellular signaling cascade /// | 200576 |
| | | | intracellular signaling cascade /// | |
| | | | calcium-mediated signaling /// | |
| | | | cellular protein metabolic process /// | |
| | | | phosphatidylinositol metabolic | |
| | | | process | |
| 32218_at | −1.090595 | — | — | — |
| 32238_at | 1.432857 | BIN1 | ATP biosynthetic process /// | 274 |
| | | | transport /// ion transport /// | |
| | | | endocytosis /// cell cycle /// | |
| | | | multicellular organismal | |
| | | | development /// cell proliferation /// | |
| | | | ATP synthesis coupled proton | |
| | | | transport /// proton transport /// | |
| | | | regulation of endocytosis /// cell | |
| | | | differentiation /// negative regulation | |
| | | | of cell cycle | |
| 32340_s_at | −1.66312 | YBX1 | transcription /// regulation of | 4904 |
| | | | transcription, DNA-dependent /// | |
| | | | regulation of transcription, DNA- | |
| | | | dependent /// transcription from RNA | |
| | | | polymerase II promoter /// mRNA | |
| | | | processing /// RNA splicing | |
| 32828_at | −2.216035 | BCKDK | signal transduction /// amino acid | 10295 |
| | | | catabolic process /// branched chain | |
| | | | family amino acid catabolic process | |
| | | | /// branched chain family amino acid | |
| | | | catabolic process /// phosphorylation | |
| | | | /// phosphorylation /// | |
| | | | phosphorylation /// peptidyl-histidine | |
| | | | phosphorylation | |
| 33176_at | −1.302196 | DOHH | peptidyl-lysine modification to | 83475 |
| | | | hypusine /// peptidyl-lysine | |
| | | | modification to hypusine | |
| 33204_at | −0.607135 | FOXD1 | transcription /// regulation of | 2297 |
| | | | transcription, DNA-dependent | |
| 33388_at | 2.300796 | TEX261 | — | 113419 |
| 33444_at | −2.738947 | NBR1 /// | — | 100133166 |
| | | LOC100133166 | | /// 4077 |
| 34523_at | −2.692606 | APOA4 | innate immune response in mucosa | 337 |
| | | | /// transport /// lipid transport /// lipid | |
| | | | transport /// response to lipid | |
| | | | hydroperoxide /// leukocyte adhesion | |
| | | | /// cholesterol metabolic process /// | |
| | | | removal of superoxide radicals /// | |
| | | | regulation of cholesterol transport /// | |
| | | | cholesterol efflux /// phospholipid | |
| | | | efflux /// lipoprotein metabolic | |
| | | | process /// lipoprotein modification /// | |
| | | | cholesterol homeostasis /// hydrogen | |
| | | | peroxide catabolic process /// | |
| | | | reverse cholesterol transport /// | |
| | | | multicellular organismal lipid | |
| | | | catabolic process /// | |
| | | | phosphatidylcholine metabolic | |
| | | | process /// lipid homeostasis /// | |
| | | | protein-lipid complex assembly | |
| 34647_at | 3.145899 | DDX5 | mRNA processing /// RNA splicing /// | 1655 |
| | | | cell growth | |
| 34773_at | 1.963235 | TBCA | tubulin folding /// post-chaperonin | 6902 |
| | | | tubulin folding pathway /// beta- | |
| | | | tubulin folding | |
| 34801_at | −1.144803 | PAN2 | nuclear-transcribed mRNA catabolic | 9924 |
| | | | process, nonsense-mediated decay | |
| | | | /// ubiquitin-dependent protein | |
| | | | catabolic process | |
| 34804_at | −0.852211 | SLC25A36 | transport /// mitochondrial transport | 55186 |
| 35018_at | −1.802839 | CHP | potassium ion transport /// small | 11261 |
| | | | GTPase mediated signal | |
| | | | transduction | |
| 35655_at | −1.746975 | ANKRD28 | — | 23243 |
| 35714_at | −2.095963 | PDXK | pyridoxine biosynthetic process | 8566 |
| 35770_at | 3.159298 | ATP6AP1 | protein import into nucleus, | 537 |
| | | | translocation /// ATP biosynthetic | |
| | | | process /// transport /// ion transport | |
| | | | /// response to stress /// fibroblast | |
| | | | growth factor receptor signaling | |
| | | | pathway /// ATP synthesis coupled | |
| | | | proton transport /// proton transport | |
| | | | /// proton transport /// regulation of | |
| | | | transcription /// positive regulation of | |
| | | | receptor-mediated endocytosis /// | |
| | | | positive regulation of urothelial cell | |
| | | | proliferation | |
| 35815_at | −2.192203 | SETD2 | transcription /// regulation of | 29072 |
| | | | transcription, DNA-dependent /// | |
| | | | chromatin modification | |
| 36068_at | −1.112913 | CCS | superoxide metabolic process /// | 9973 |
| | | | superoxide metabolic process /// | |
| | | | intracellular copper ion transport /// | |
| | | | metal ion transport /// positive | |
| | | | regulation of oxidoreductase activity | |
| 36209_at | −2.145239 | BRD2 | spermatogenesis | 6046 |
| 36250_at | −1.191006 | ASPHD1 | peptidyl-amino acid modification | 253982 |
| 36366_at | 1.997465 | B4GALT6 | carbohydrate metabolic process | 9331 |
| 36395_at | 2.355726 | — | — | — |
| 36528_at | 0.743151 | ASL | urea cycle /// argininosuccinate | 435 |
| | | | metabolic process /// response to | |
| | | | hypoxia /// kidney development /// | |
| | | | liver development /// arginine | |
| | | | biosynthetic process /// arginine | |
| | | | catabolic process /// response to | |
| | | | nutrient /// amino acid biosynthetic | |
| | | | process /// arginine biosynthetic | |
| | | | process via ornithine /// response to | |
| | | | peptide hormone stimulus /// | |
| | | | response to glucocorticoid stimulus | |
| | | | /// response to cAMP | |
| 36641_at | −0.973701 | CAPZA2 | protein complex assembly /// cell | 830 |
| | | | motility /// actin cytoskeleton | |
| | | | organization and biogenesis /// | |
| | | | barbed-end actin filament capping | |
| 37355_at | −3.431367 | STARD3 | lipid metabolic process /// steroid | 10948 |
| | | | biosynthetic process /// C21-steroid | |
| | | | hormone biosynthetic process /// | |
| | | | transport /// mitochondrial transport | |
| | | | /// lipid transport /// steroid metabolic | |
| | | | process /// cholesterol metabolic | |
| | | | process | |
| 38618_at | 0.761043 | LIMK2 /// | protein amino acid phosphorylation | 3985 /// |
| | | PPP1R14BP1 | /// phosphorylation /// regulation of | 50516 |
| | | | phosphorylation | |
| 38663_at | −0.841092 | BANF1 | response to virus | 8815 |
| 38831_f_at | −1.128399 | GNB2 | signal transduction /// G-protein | 2783 |
| | | | coupled receptor protein signaling | |
| | | | pathway | |
| 39012_g_at | −1.366345 | ENSA | transport /// response to nutrient | 2029 |
| 39159_at | −0.871773 | SH3GL1 | endocytosis /// signal transduction /// | 6455 |
| | | | central nervous system development | |
| 39199_at | −1.58861 | ACVR1B | G1/S transition of mitotic cell cycle | 91 |
| | | | /// in utero embryonic development | |
| | | | /// hair follicle development /// protein | |
| | | | amino acid phosphorylation /// | |
| | | | protein amino acid phosphorylation | |
| | | | /// induction of apoptosis /// signal | |
| | | | transduction /// transmembrane | |
| | | | receptor protein serine/threonine | |
| | | | kinase signaling pathway /// | |
| | | | embryonic development /// negative | |
| | | | regulation of cell growth /// positive | |
| | | | regulation of activin receptor | |
| | | | signaling pathway /// regulation of | |
| | | | transcription /// positive regulation of | |
| | | | erythrocyte differentiation | |
| 39599_at | 1.466566 | SLC6A1 | transport /// transport /// | 6529 |
| | | | neurotransmitter transport /// | |
| | | | synaptic transmission | |
| 40867_at | 2.484647 | PPP2R1A | inactivation of MAPK activity /// | 5518 |
| | | | regulation of DNA replication /// | |
| | | | protein complex assembly /// protein | |
| | | | amino acid dephosphorylation /// | |
| | | | ceramide metabolic process /// | |
| | | | induction of apoptosis /// RNA | |
| | | | splicing /// response to organic | |
| | | | substance /// second-messenger- | |
| | | | mediated signaling /// regulation of | |
| | | | Wnt receptor signaling pathway /// | |
| | | | regulation of cell adhesion /// | |
| | | | negative regulation of cell growth /// | |
| | | | regulation of growth /// negative | |
| | | | regulation of tyrosine | |
| | | | phosphorylation of Stat3 protein /// | |
| | | | regulation of transcription /// | |
| | | | regulation of cell differentiation | |
| 41063_g_at | 1.696948 | PCGF1 | transcription /// regulation of | 84759 |
| | | | transcription, DNA-dependent | |
| 41077_at | 2.21384 | LOC643641 | transcription /// regulation of | 643641 |
| | | | transcription, DNA-dependent | |
| 41285_at | 0.839605 | INPP5A | cell communication | 3632 |
| 41489_at | −1.356162 | TLE1 | transcription /// regulation of | 7088 |
| | | | transcription, DNA-dependent /// | |
| | | | signal transduction /// multicellular | |
| | | | organismal development /// organ | |
| | | | morphogenesis /// Wnt receptor | |
| | | | signaling pathway /// negative | |
| | | | regulation of transcription /// | |
| | | | negative regulation of Wnt receptor | |
| | | | signaling pathway /// regulation of | |
| | | | transcription | |
| 41689_at | 1.121172 | PLLP | transport /// ion transport | 51090 |
| 41713_at | −1.680428 | ZKSCAN1 | transcription /// regulation of | 7586 |
| | | | transcription, DNA-dependent /// | |
| | | | regulation of transcription, DNA- | |
| | | | dependent | |
| 41762_at | 2.014074 | TIAL1 | regulation of transcription from RNA | 7073 |
| | | | polymerase II promoter /// apoptosis | |
| | | | /// induction of apoptosis /// defense | |
| | | | response | |
| 910_at | −1.438133 | TK1 | nucleobase, nucleoside, nucleotide | 7083 |
| | | | and nucleic acid metabolic process | |
| | | | /// DNA replication | |
| 922_at | 0.851269 | PPP2R1A | inactivation of MAPK activity /// | 5518 |
| | | | regulation of DNA replication /// | |
| | | | protein complex assembly /// protein | |
| | | | amino acid dephosphorylation /// | |
| | | | ceramide metabolic process /// | |
| | | | induction of apoptosis /// RNA | |
| | | | splicing /// response to organic | |
| | | | substance /// second-messenger- | |
| | | | mediated signaling /// regulation of | |
| | | | Wnt receptor signaling pathway /// | |
| | | | regulation of cell adhesion /// | |
| | | | negative regulation of cell growth /// | |
| | | | regulation of growth /// negative | |
| | | | regulation of tyrosine | |
| | | | phosphorylation of Stat3 protein /// | |
| | | | regulation of transcription /// | |
| | | | regulation of cell differentiation | |
| 941_at | 2.247213 | PSMB6 | ubiquitin-dependent protein | 5694 |
| | | | catabolic process /// ubiquitin- | |
| | | | dependent protein catabolic process | |
| 954_s_at | −1.514611 | — | — | — |

TABLE 5
Etoposide responsivity predictor set

| Etopo | | | | |
|---|---|---|---|---|
| Probe Set | | | | Entrez |
| ID | Weight | Gene Symbol | Go biological process term | Gene ID |
| 1015_s_at | 1.784401 | LIMK1 | protein amino acid phosphorylation /// | 3984 |
| | | | protein amino acid phosphorylation /// | |
| | | | cell motility /// signal transduction /// | |
| | | | Rho protein signal transduction /// | |
| | | | nervous system development /// actin | |
| | | | cytoskeleton organization and | |
| | | | biogenesis /// positive regulation of | |
| | | | axon extension | |
| 1188_g_at | −1.618287 | LIG3 | DNA replication /// DNA repair /// DNA | 3980 |
| | | | repair /// DNA recombination /// | |
| | | | response to DNA damage stimulus /// | |
| | | | cell cycle /// meiotic recombination /// | |
| | | | spermatogenesis /// V(D)J | |
| | | | recombination /// cell division | |
| 1233_s_at | −1.351889 | AXL | protein amino acid phosphorylation /// | 558 |
| | | | signal transduction | |
| 1456_s_at | −1.981805 | IFI16 | transcription /// regulation of | 3428 |
| | | | transcription, DNA-dependent /// | |
| | | | regulation of transcription, DNA- | |
| | | | dependent /// cell proliferation /// | |
| | | | response to virus /// hemopoiesis /// | |
| | | | myeloid cell differentiation /// monocyte | |
| | | | differentiation /// DNA damage | |
| | | | response, signal transduction by p53 | |
| | | | class mediator resulting in induction of | |
| | | | apoptosis | |
| 160020_at | 1.795838 | MMP14 | ossification /// angiogenesis /// ovarian | 4323 |
| | | | follicle development /// response to | |
| | | | hypoxia /// endothelial cell proliferation | |
| | | | /// proteolysis /// proteolysis /// | |
| | | | response to oxidative stress /// | |
| | | | metabolic process /// response to | |
| | | | mechanical stimulus /// response to | |
| | | | hormone stimulus /// cell migration /// | |
| | | | lung development /// zymogen | |
| | | | activation /// astrocyte cell migration /// | |
| | | | response to estrogen stimulus /// | |
| | | | branching morphogenesis of a tube /// | |
| | | | tissue remodeling /// negative | |
| | | | regulation of focal adhesion formation | |
| 1680_at | −4.528865 | GRB7 | signal transduction /// epidermal | 2886 |
| | | | growth factor receptor signaling | |
| | | | pathway | |
| 1704_at | −0.820263 | VAV2 | signal transduction /// intracellular | 7410 |
| | | | signaling cascade /// small GTPase | |
| | | | mediated signal transduction /// cell | |
| | | | migration /// lamellipodium biogenesis | |
| | | | /// regulation of Rho protein signal | |
| | | | transduction /// positive regulation of | |
| | | | phosphoinositide 3-kinase activity | |
| 1963_at | −1.84567 | FLT1 | angiogenesis /// patterning of blood | 2321 |
| | | | vessels /// protein amino acid | |
| | | | phosphorylation /// transmembrane | |
| | | | receptor protein tyrosine kinase | |
| | | | signaling pathway /// transmembrane | |
| | | | receptor protein tyrosine kinase | |
| | | | signaling pathway /// multicellular | |
| | | | organismal development /// female | |
| | | | pregnancy /// positive regulation of cell | |
| | | | proliferation /// cell migration /// cell | |
| | | | differentiation /// vascular endothelial | |
| | | | growth factor receptor signaling | |
| | | | pathway | |
| 2047_s_at | 3.095477 | JUP | cell adhesion /// cell adhesion /// cell- | 3728 |
| | | | cell adhesion | |
| 296_at | −2.126599 | — | — | — |
| 297_g_at | −1.354399 | — | — | — |
| 311_s_at | 0.902404 | — | — | — |
| 31719_at | −0.768085 | FN1 | acute-phase response /// cell adhesion | 2335 |
| | | | /// cell adhesion /// transmembrane | |
| | | | receptor protein tyrosine kinase | |
| | | | signaling pathway /// metabolic | |
| | | | process /// response to wounding /// | |
| | | | cell migration | |
| 31720_s_at | −0.997247 | FN1 | acute-phase response /// cell adhesion | 2335 |
| | | | /// cell adhesion /// transmembrane | |
| | | | receptor protein tyrosine kinase | |
| | | | signaling pathway /// metabolic | |
| | | | process /// response to wounding /// | |
| | | | cell migration | |
| 32378_at | −1.419169 | PKM2 | glycolysis /// glycolysis | 5315 |
| 32387_at | −2.0891 | LYPLA3 | lipid metabolic process /// fatty acid | 23659 |
| | | | metabolic process /// ceramide | |
| | | | metabolic process /// fatty acid | |
| | | | catabolic process | |
| 32593_at | 1.245739 | RFTN1 | — | 23180 |
| 33282_at | −1.860632 | LAD1 | — | 3898 |
| 33448_at | 2.351885 | SPINT1 | morphogenesis of a branching | 6692 |
| | | | structure /// embryonic placenta | |
| | | | development | |
| 33904_at | −5.280012 | CLDN3 | response to hypoxia /// calcium- | 1365 |
| | | | independent cell-cell adhesion /// | |
| | | | calcium-independent cell-cell adhesion | |
| 34320_at | 0.752612 | PTRF | transcription /// transcription | 284119 |
| | | | termination /// regulation of | |
| | | | transcription, DNA-dependent /// | |
| | | | transcription initiation from RNA | |
| | | | polymerase I promoter | |
| 34348_at | −4.77987 | SPINT2 /// | cell motility | 100130414 |
| | | LOC100130414 | | /// 10653 |
| 34747_at | −1.301025 | MMP14 | ossification /// angiogenesis /// ovarian | 4323 |
| | | | follicle development /// response to | |
| | | | hypoxia /// endothelial cell proliferation | |
| | | | /// proteolysis /// proteolysis /// | |
| | | | response to oxidative stress /// | |
| | | | metabolic process /// response to | |
| | | | mechanical stimulus /// response to | |
| | | | hormone stimulus /// cell migration /// | |
| | | | lung development /// zymogen | |
| | | | activation /// astrocyte cell migration /// | |
| | | | response to estrogen stimulus /// | |
| | | | branching morphogenesis of a tube /// | |
| | | | tissue remodeling /// negative | |
| | | | regulation of focal adhesion formation | |
| 34769_at | −0.957897 | FAAH | fatty acid metabolic process | 2166 |
| 35276_at | −0.982402 | CLDN4 | pathogenesis /// calcium-independent | 1364 |
| | | | cell-cell adhesion | |
| 35309_at | 2.289795 | ST14 | proteolysis /// proteolysis | 6768 |
| 35444_at | 1.213463 | C19orf21 | — | 126353 |
| 35541_r_at | 0.994896 | KIAA0506 | — | 57239 |
| 35630_at | 1.157887 | LLGL2 | cell cycle /// cell division | 3993 |
| 35669_at | −5.408485 | COBL | — | 23242 |
| 35681_r_at | 0.991531 | ZEB2 | transcription /// regulation of | 9839 |
| | | | transcription, DNA-dependent /// | |
| | | | nervous system development /// | |
| | | | negative regulation of transcription /// | |
| | | | regulation of transcription | |
| 35735_at | −0.843365 | GBP1 | immune response | 2633 |
| 36097_at | 2.89579 | IER2 | — | 9592 |
| 36890_at | −1.433033 | PPL | keratinization | 5493 |
| 37934_at | 1.072557 | TMEM30B | — | 161291 |
| 38221_at | 0.839583 | CNKSR1 | transmembrane receptor protein | 10256 |
| | | | tyrosine kinase signaling pathway /// | |
| | | | Ras protein signal transduction /// Rho | |
| | | | protein signal transduction | |
| 38482_at | 3.716125 | CLDN7 | calcium-independent cell-cell adhesion | 1366 |
| 38759_at | 0.944703 | BTN3A2 | — | 11118 |
| 38760_f_at | −1.596566 | BTN3A2 | — | 11118 |
| 39331_at | 2.411297 | TUBB2A | microtubule-based process /// | 7280 |
| | | | microtubule-based movement /// | |
| | | | mitosis /// neuron differentiation /// | |
| | | | protein polymerization | |
| 39732_at | 2.325338 | MAP7 | microtubule cytoskeleton organization | 9053 |
| | | | and biogenesis /// establishment | |
| | | | and/or maintenance of cell polarity | |
| 39870_at | −1.522243 | RBMXL2 | — | 27288 |
| 40215_at | 1.420872 | UGCG | glucosylceramide biosynthetic process | 7357 |
| | | | /// glycosphingolipid biosynthetic | |
| | | | process /// epidermis development | |
| 40225_at | 2.377423 | GAK | protein amino acid phosphorylation /// | 2580 |
| | | | cell cycle | |
| 41359_at | 0.513065 | PKP3 | cell adhesion | 11187 |
| 41872_at | 0.58884 | DFNA5 | sensory perception of sound /// | 1687 |
| | | | sensory perception of sound /// inner | |
| | | | ear receptor cell differentiation | |
| 479_at | −1.886719 | DAB2 | cell proliferation | 1601 |
| 575_s_at | 3.082061 | TACSTD1 | — | 4072 |
| 671_at | 1.491038 | SPARC | ossification /// transmembrane receptor | 6678 |
| | | | protein tyrosine kinase signaling | |
| | | | pathway | |
| 903_at | −2.447505 | PPP2R5A | signal transduction | 5525 |

TABLE 6
Taxol responsivity predictor set

| Taxol | | | | |
|---|---|---|---|---|
| Probe Set | | | | Entrez |
| ID | Weight | Gene Symbol | Go biological process term | Gene ID |
| 1218_at | 2.174617 | NR2F6 | transcription /// regulation of | 2063 |
| | | | transcription, DNA-dependent /// | |
| | | | signal transduction /// entrainment of | |
| | | | circadian clock by photoperiod /// | |
| | | | neuron development /// detection of | |
| | | | temperature stimulus involved in | |
| | | | sensory perception of pain | |
| 1581_s_at | −1.252126 | TOP2B | neuron migration /// DNA metabolic | 7155 |
| | | | process /// DNA topological change | |
| | | | /// axonogenesis /// forebrain | |
| | | | development | |
| 1587_at | 1.902324 | RARG | transcription /// regulation of | 5916 |
| | | | transcription, DNA-dependent /// | |
| | | | multicellular organismal | |
| | | | development | |
| 1824_s_at | 1.645376 | PCNA | DNA replication /// DNA replication | 5111 |
| | | | /// regulation of DNA replication /// | |
| | | | DNA repair /// base-excision repair, | |
| | | | gap-filling /// intracellular protein | |
| | | | transport /// cell proliferation /// | |
| | | | phosphoinositide-mediated signaling | |
| 1871_g_at | 1.490669 | PTPN11 | protein amino acid | 5781 |
| | | | dephosphorylation /// signal | |
| | | | transduction /// sensory perception | |
| | | | of sound /// dephosphorylation | |
| 1882_g_at | 1.181144 | — | — | — |
| 1903_at | 2.373819 | — | — | — |
| 2001_g_at | −1.02787 | ATM | DNA repair /// DNA repair /// | 472 |
| | | | response to DNA damage stimulus | |
| | | | /// cell cycle /// mitotic cell cycle | |
| | | | spindle assembly checkpoint /// | |
| | | | meiotic recombination /// signal | |
| | | | transduction /// response to ionizing | |
| | | | radiation /// negative regulation of | |
| | | | cell cycle | |
| 249_at | 2.686971 | NFATC4 | transcription /// regulation of | 4776 |
| | | | transcription, DNA-dependent /// | |
| | | | transcription from RNA polymerase | |
| | | | II promoter /// inflammatory response | |
| | | | /// heart development /// cellular | |
| | | | respiration /// regulation of | |
| | | | transcription | |
| 32386_at | 0.851159 | LOC100130134 | — | 100130134 |
| 33064_at | −2.893535 | CACNG1 | transport /// transport /// ion transport | 786 |
| | | | /// calcium ion transport /// muscle | |
| | | | contraction | |
| 33557_at | 2.052513 | C22orf31 | — | 25770 |
| 335_r_at | −2.423531 | — | — | — |
| 34197_at | 1.523938 | PIK3R2 | signal transduction /// negative | 5296 |
| | | | regulation of anti-apoptosis /// | |
| | | | negative regulation of anti-apoptosis | |
| 34247_at | 2.406482 | — | — | — |
| 34471_at | 2.325227 | MYH8 | muscle contraction /// striated | 4626 |
| | | | muscle contraction | |
| 34862_at | 0.634438 | SCCPDH | metabolic process | 51097 |
| 34909_at | −1.087802 | PHTF2 | transcription /// regulation of | 57157 |
| | | | transcription, DNA-dependent | |
| 34923_at | 2.607029 | IQSEC2 | regulation of ARF protein signal | 23096 |
| | | | transduction | |
| 34984_at | 3.099121 | TRPC3 | transport /// ion transport /// calcium | 7222 |
| | | | ion transport /// calcium ion transport | |
| | | | /// phototransduction | |
| 35254_at | 0.711369 | TRAFD1 | — | 10906 |
| 35644_at | 4.69153 | HEPH | transport /// ion transport /// copper | 9843 |
| | | | ion transport /// iron ion transport | |
| 35908_at | −1.593513 | SOX30 | transcription /// regulation of | 11063 |
| | | | transcription, DNA-dependent | |
| 36595_s_at | 1.499879 | GATM | creatine biosynthetic process | 2628 |
| 37378_r_at | −1.0972 | LMNA | — | 4000 |
| 37767_at | 2.505571 | HTT | apoptosis /// apoptosis /// induction | 3064 |
| | | | of apoptosis /// behavior /// | |
| | | | pathogenesis /// organ | |
| | | | morphogenesis | |
| 38680_at | 1.486391 | SNRPE | spliceosome assembly /// mRNA | 6635 |
| | | | processing /// RNA splicing /// mRNA | |
| | | | metabolic process | |
| 38697_at | −2.522602 | YIPF3 | cell differentiation | 25844 |
| 38703_at | −0.908363 | DNPEP | proteolysis /// peptide metabolic | 23549 |
| | | | process | |
| 39488_at | 1.644714 | PCDH9 | cell adhesion /// homophilic cell | 5101 |
| | | | adhesion | |
| 39537_at | −3.282921 | KLHDC3 | ossification /// transport /// Golgi to | 116138 |
| | | | endosome transport /// endocytosis | |
| | | | /// endocytosis /// meiosis /// meiotic | |
| | | | recombination /// G-protein coupled | |
| | | | receptor protein signaling pathway /// | |
| | | | neuropeptide signaling pathway /// | |
| | | | multicellular organismal | |
| | | | development /// endosome to | |
| | | | lysosome transport /// induction of | |
| | | | apoptosis by extracellular signals /// | |
| | | | regulation of gene expression /// | |
| | | | myotube differentiation /// vesicle | |
| | | | organization and biogenesis /// cell | |
| | | | differentiation /// endosome transport | |
| | | | via multivesicular body sorting | |
| | | | pathway /// response to insulin | |
| | | | stimulus /// negative regulation of | |
| | | | apoptosis /// glucose import /// nerve | |
| | | | growth factor receptor signaling | |
| | | | pathway /// plasma membrane to | |
| | | | endosome transport /// negative | |
| | | | regulation of lipoprotein lipase | |
| | | | activity | |
| 40360_at | 1.718898 | SLC10A3 | transport /// sodium ion transport /// | 8273 |
| | | | sodium ion transport /// organic | |
| | | | anion transport | |
| 40529_at | −1.361106 | LHX2 | transcription /// regulation of | 9355 |
| | | | transcription, DNA-dependent /// | |
| | | | nervous system development /// | |
| | | | brain development /// mesoderm | |
| | | | development /// dorsal/ventral | |
| | | | pattern formation /// regulation of | |
| | | | transcription | |
| 40690_at | 0.503167 | CKS2 | regulation of cyclin-dependent | 1164 |
| | | | protein kinase activity /// cell cycle /// | |
| | | | cell cycle /// spindle organization and | |
| | | | biogenesis /// meiosis I /// cell | |
| | | | proliferation /// phosphoinositide- | |
| | | | mediated signaling /// cell division | |
| 41045_at | 0.75712 | SECTM1 | immune response /// mesoderm | 6398 |
| | | | development /// positive regulation of | |
| | | | I-kappaB kinase/NF-kappaB | |
| | | | cascade | |
| 41204_s_at | −1.781384 | SF1 | spliceosome assembly /// nuclear | 7536 |
| | | | mRNA 3′-splice site recognition /// | |
| | | | nuclear mRNA splicing, via | |
| | | | spliceosome /// transcription /// | |
| | | | regulation of transcription, DNA- | |
| | | | dependent /// mRNA processing /// | |
| | | | RNA splicing /// negative regulation | |
| | | | of smooth muscle cell proliferation | |
| 41404_at | −1.779211 | RPS6KA4 | regulation of transcription, DNA- | 8986 |
| | | | dependent /// protein amino acid | |
| | | | phosphorylation /// protein amino | |
| | | | acid phosphorylation /// protein | |
| | | | amino acid phosphorylation /// | |
| | | | protein kinase cascade /// protein | |
| | | | kinase cascade | |
| 761_g_at | −2.000734 | DYRK2 | protein amino acid phosphorylation | 8445 |
| | | | /// protein amino acid | |
| | | | phosphorylation /// protein amino | |
| | | | acid phosphorylation /// apoptosis /// | |
| | | | DNA damage response, signal | |
| | | | transduction by p53 class mediator | |
| | | | resulting in induction of apoptosis /// | |
| | | | positive regulation of glycogen | |
| | | | biosynthetic process | |
| 777_at | 0.746673 | GDI2 | signal transduction /// protein | 2665 |
| | | | transport /// regulation of GTPase | |
| | | | activity | |
| 925_at | −1.943148 | PIK3R2 /// | signal transduction /// negative | 10437 /// |
| | | IFI30 | regulation of anti-apoptosis /// | 5296 |
| | | | negative regulation of anti-apoptosis | |

TABLE 7
Topotecan responsivity predictor set

| Topo Probe | | | | Entrez |
|---|---|---|---|---|
| Set ID | Weight | Gene Symbol | Go biological process term | Gene ID |
| 1005_at | −1.583466 | DUSP1 | protein amino acid | 1843 |
| | | | dephosphorylation /// response to | |
| | | | stress /// response to oxidative | |
| | | | stress /// cell cycle /// intracellular | |
| | | | signaling cascade /// | |
| | | | dephosphorylation | |
| 115_at | −0.533912 | THBS1 | cell motility /// cell adhesion /// | 7057 |
| | | | multicellular organismal | |
| | | | development /// nervous system | |
| | | | development /// blood coagulation | |
| 1233_s_at | 0.416455 | AXL | protein amino acid phosphorylation | 558 |
| | | | /// signal transduction | |
| 1251_g_at | −2.051381 | RAP1GAP | signal transduction /// signal | 5909 |
| | | | transduction /// signal transduction | |
| | | | /// regulation of small GTPase | |
| | | | mediated signal transduction | |
| 1257_s_at | −0.915209 | QSOX1 | protein thiol-disulfide exchange /// | 5768 |
| | | | negative regulation of cell | |
| | | | proliferation /// cell redox | |
| | | | homeostasis | |
| 1278_at | −1.053777 | — | — | — |
| 1368_at | 0.95653 | IL1R1 | inflammatory response /// immune | 3554 |
| | | | response /// signal transduction /// | |
| | | | cell surface receptor linked signal | |
| | | | transduction /// cytokine and | |
| | | | chemokine mediated signaling | |
| | | | pathway /// innate immune | |
| | | | response | |
| 1385_at | −0.254775 | TGFBI | cell adhesion /// negative | 7045 |
| | | | regulation of cell adhesion /// visual | |
| | | | perception /// visual perception /// | |
| | | | cell proliferation /// response to | |
| | | | stimulus | |
| 1491_at | −1.32518 | PTX3 | response to yeast /// inflammatory | 5806 |
| | | | response /// opsonization /// | |
| | | | positive regulation of nitric oxide | |
| | | | biosynthetic process /// positive | |
| | | | regulation of phagocytosis | |
| 1544_at | −2.317916 | BLM | regulation of cyclin-dependent | 641 |
| | | | protein kinase activity /// G2 phase | |
| | | | of mitotic cell cycle /// telomere | |
| | | | maintenance /// double-strand | |
| | | | break repair via homologous | |
| | | | recombination /// DNA replication | |
| | | | /// DNA repair /// DNA repair /// | |
| | | | DNA recombination /// DNA | |
| | | | recombination /// response to DNA | |
| | | | damage stimulus /// response to X- | |
| | | | ray /// G2/M transition DNA | |
| | | | damage checkpoint /// cellular | |
| | | | metabolic process /// negative | |
| | | | regulation of DNA recombination /// | |
| | | | positive regulation of transcription | |
| | | | /// negative regulation of mitotic | |
| | | | recombination /// alpha-beta T cell | |
| | | | differentiation /// positive regulation | |
| | | | of alpha-beta T cell proliferation /// | |
| | | | replication fork protection /// | |
| | | | regulation of binding /// protein | |
| | | | oligomerization /// chromosome | |
| | | | organization and biogenesis /// | |
| | | | negative regulation of cell division | |
| 1563_s_at | −1.13338 | TNFRSF1A | prostaglandin metabolic process /// | 7132 |
| | | | apoptosis /// inflammatory | |
| | | | response /// inflammatory | |
| | | | response /// signal transduction /// | |
| | | | cytokine and chemokine mediated | |
| | | | signaling pathway /// cytokine and | |
| | | | chemokine mediated signaling | |
| | | | pathway /// positive regulation of I- | |
| | | | kappaB kinase/NF-kappaB | |
| | | | cascade /// positive regulation of | |
| | | | transcription from RNA polymerase | |
| | | | II promoter /// positive regulation of | |
| | | | transcription from RNA polymerase | |
| | | | II promoter /// positive regulation of | |
| | | | inflammatory response /// positive | |
| | | | regulation of inflammatory | |
| | | | response | |
| 1593_at | −0.931912 | FGF2 | activation of MAPKK activity /// | 2247 |
| | | | activation of MAPK activity /// | |
| | | | angiogenesis /// induction of an | |
| | | | organ /// positive regulation of | |
| | | | protein amino acid phosphorylation | |
| | | | /// chemotaxis /// signal | |
| | | | transduction /// Ras protein signal | |
| | | | transduction /// cell-cell signaling /// | |
| | | | multicellular organismal | |
| | | | development /// nervous system | |
| | | | development /// muscle | |
| | | | development /// cell proliferation /// | |
| | | | positive regulation of cell | |
| | | | proliferation /// negative regulation | |
| | | | of cell proliferation /// organ | |
| | | | morphogenesis /// glial cell | |
| | | | differentiation /// positive regulation | |
| | | | of granule cell precursor | |
| | | | proliferation /// cell differentiation /// | |
| | | | lung development /// positive | |
| | | | regulation of cell differentiation /// | |
| | | | positive regulation of angiogenesis | |
| | | | /// regulation of retinal cell | |
| | | | programmed cell death /// positive | |
| | | | regulation of epithelial cell | |
| | | | proliferation | |
| 159_at | −1.131544 | VEGFC | angiogenesis /// positive regulation | 7424 |
| | | | of neuroblast proliferation /// | |
| | | | substrate-bound cell migration /// | |
| | | | signal transduction /// multicellular | |
| | | | organismal development /// cell | |
| | | | proliferation /// positive regulation | |
| | | | of cell proliferation /// positive | |
| | | | regulation of cell proliferation /// | |
| | | | organ morphogenesis /// | |
| | | | morphogenesis of embryonic | |
| | | | epithelium /// cell differentiation /// | |
| | | | vascular endothelial growth factor | |
| | | | receptor signaling pathway | |
| 160044_g_at | −1.330621 | ACO2 | generation of precursor | 50 |
| | | | metabolites and energy /// | |
| | | | tricarboxylic acid cycle /// | |
| | | | tricarboxylic acid cycle /// citrate | |
| | | | metabolic process /// citrate | |
| | | | metabolic process /// metabolic | |
| | | | process | |
| 1751_g_at | −1.870035 | FARSA | translation /// tRNA aminoacylation | 2193 |
| | | | for protein translation /// | |
| | | | phenylalanyl-tRNA aminoacylation | |
| 1783_at | 0.658517 | RIN2 | endocytosis /// signal transduction | 54453 |
| | | | /// small GTPase mediated signal | |
| | | | transduction | |
| 1828_s_at | −1.142258 | FGF2 | activation of MAPKK activity /// | 2247 |
| | | | activation of MAPK activity /// | |
| | | | angiogenesis /// induction of an | |
| | | | organ /// positive regulation of | |
| | | | protein amino acid phosphorylation | |
| | | | /// chemotaxis /// signal | |
| | | | transduction /// Ras protein signal | |
| | | | transduction /// cell-cell signaling /// | |
| | | | multicellular organismal | |
| | | | development /// nervous system | |
| | | | development /// muscle | |
| | | | development /// cell proliferation /// | |
| | | | positive regulation of cell | |
| | | | proliferation /// negative regulation | |
| | | | of cell proliferation /// organ | |
| | | | morphogenesis /// glial cell | |
| | | | differentiation /// positive regulation | |
| | | | of granule cell precursor | |
| | | | proliferation /// cell differentiation /// | |
| | | | lung development /// positive | |
| | | | regulation of cell differentiation /// | |
| | | | positive regulation of angiogenesis | |
| | | | /// regulation of retinal cell | |
| | | | programmed cell death /// positive | |
| | | | regulation of epithelial cell | |
| | | | proliferation | |
| 1879_at | −1.345807 | RRAS | small GTPase mediated signal | 6237 |
| | | | transduction /// Ras protein signal | |
| | | | transduction /// negative regulation | |
| | | | of cell migration | |
| 1958_at | −0.849681 | FIGF | angiogenesis /// multicellular | 2277 |
| | | | organismal development /// cell | |
| | | | proliferation /// positive regulation | |
| | | | of cell proliferation /// positive | |
| | | | regulation of cell proliferation /// | |
| | | | cell differentiation /// vascular | |
| | | | endothelial growth factor receptor | |
| | | | signaling pathway | |
| 2042_s_at | −2.085535 | MYB | transcription /// regulation of | 4602 |
| | | | transcription, DNA-dependent /// | |
| | | | regulation of transcription, DNA- | |
| | | | dependent /// regulation of | |
| | | | transcription | |
| 2053_at | −1.376639 | CDH2 | cell adhesion /// cell adhesion /// | 1000 |
| | | | homophilic cell adhesion | |
| 2056_at | −1.331695 | FGFR1 | MAPKKK cascade /// skeletal | 2260 |
| | | | development /// protein amino acid | |
| | | | phosphorylation /// protein amino | |
| | | | acid phosphorylation /// fibroblast | |
| | | | growth factor receptor signaling | |
| | | | pathway /// fibroblast growth factor | |
| | | | receptor signaling pathway /// cell | |
| | | | growth | |
| 2057_g_at | −1.931123 | FGFR1 | MAPKKK cascade /// skeletal | 2260 |
| | | | development /// protein amino acid | |
| | | | phosphorylation /// protein amino | |
| | | | acid phosphorylation /// fibroblast | |
| | | | growth factor receptor signaling | |
| | | | pathway /// fibroblast growth factor | |
| | | | receptor signaling pathway /// cell | |
| | | | growth | |
| 232_at | −1.506829 | LAMC1 | protein complex assembly /// cell | 3915 |
| | | | adhesion /// cell adhesion /// | |
| | | | endoderm development /// cell | |
| | | | migration /// extracellular matrix | |
| | | | disassembly /// hemidesmosome | |
| | | | assembly /// positive regulation of | |
| | | | epithelial cell proliferation | |
| 31521_f_at | −2.857943 | HIST1H4I /// | establishment and/or maintenance | 121504 /// |
| | | HIST1H4A /// | of chromatin architecture /// | 554313 /// |
| | | HIST1H4D /// | nucleosome assembly /// | 8294 /// |
| | | HIST1H4F /// | phosphoinositide-mediated | 8359 /// |
| | | HIST1H4K /// | signaling | 8360 /// |
| | | HIST1H4J /// | | 8361 /// |
| | | HIST1H4C /// | | 8362 /// |
| | | HIST1H4H /// | | 8363 /// |
| | | HIST1H4B /// | | 8364 /// |
| | | HIST1H4E /// | | 8365 /// |
| | | HIST1H4L /// | | 8366 /// |
| | | HIST2H4A /// | | 8367 /// |
| | | HIST4H4 /// | | 8368 /// |
| | | HIST2H4B | | 8370 |
| 32098_at | 0.474113 | COL6A2 | phosphate transport /// cell | 1292 |
| | | | adhesion /// cell-cell adhesion /// | |
| | | | extracellular matrix organization | |
| | | | and biogenesis | |
| 32116_at | −1.798732 | TMC6 | — | 11322 |
| 32260_at | 0.772105 | PEA15 | transport /// transport /// apoptosis | 8682 |
| | | | /// anti-apoptosis /// carbohydrate | |
| | | | transport /// regulation of apoptosis | |
| | | | /// regulation of apoptosis /// | |
| | | | negative regulation of glucose | |
| | | | import | |
| 32434_at | −0.868285 | MARCKS | cell motility | 4082 |
| 32529_at | −1.490314 | CKAP4 | — | 10970 |
| 32531_at | 0.748264 | GJA1 | in utero embryonic development /// | 2697 |
| | | | neuron migration /// heart looping | |
| | | | /// epithelial cell maturation /// | |
| | | | transport /// apoptosis /// muscle | |
| | | | contraction /// cell communication | |
| | | | /// cell-cell signaling /// cell-cell | |
| | | | signaling /// heart development /// | |
| | | | adult heart development /// | |
| | | | sensory perception of sound /// | |
| | | | regulation of heart contraction /// | |
| | | | negative regulation of cell | |
| | | | proliferation /// response to pH /// | |
| | | | vascular transport /// ATP transport | |
| | | | /// gap junction assembly /// | |
| | | | embryonic heart tube development | |
| | | | /// positive regulation of I-kappaB | |
| | | | kinase/NF-kappaB cascade /// | |
| | | | skeletal muscle regeneration /// | |
| | | | positive regulation of protein | |
| | | | catabolic process /// positive | |
| | | | regulation of striated muscle | |
| | | | development /// blood vessel | |
| | | | morphogenesis /// neurite | |
| | | | morphogenesis /// protein | |
| | | | oligomerization /// regulation of | |
| | | | calcium ion transport | |
| 32535_at | −0.37409 | FBN1 | skeletal development /// heart | 2200 |
| | | | development /// blood coagulation | |
| 32606_at | 0.49158 | — | — | — |
| 32607_at | −0.910611 | BASP1 | — | 10409 |
| 32673_at | −0.94355 | BTN2A1 | lipid metabolic process | 11120 |
| 32808_at | −1.041547 | ITGB1 | cellular defense response /// cell | 3688 |
| | | | adhesion /// homophilic cell | |
| | | | adhesion /// cell-matrix adhesion /// | |
| | | | integrin-mediated signaling | |
| | | | pathway /// multicellular organismal | |
| | | | development /// cell migration | |
| 32812_at | −0.762285 | LIMCH1 | actomyosin structure organization | 22998 |
| | | | and biogenesis | |
| 32847_at | −1.519068 | MYLK | protein amino acid phosphorylation | 4638 |
| | | | /// protein amino acid | |
| | | | phosphorylation | |
| 33127_at | −0.928141 | LOXL2 /// | UDP catabolic process /// protein | 4017 /// |
| | | ENTPD4 | modification process /// cell | 9583 |
| | | | adhesion /// aging | |
| 33328_at | −0.492804 | HEG1 | — | 57493 |
| 33337_at | −1.951393 | DEGS1 | lipid metabolic process /// fatty acid | 8560 |
| | | | biosynthetic process /// | |
| | | | unsaturated fatty acid biosynthetic | |
| | | | process /// lipid biosynthetic | |
| | | | process | |
| 33404_at | −1.525294 | CAP2 | cytoskeleton organization and | 10486 |
| | | | biogenesis /// establishment and/or | |
| | | | maintenance of cell polarity /// | |
| | | | signal transduction /// activation of | |
| | | | adenylate cyclase activity | |
| 33405_at | −1.580917 | CAP2 | cytoskeleton organization and | 10486 |
| | | | biogenesis /// establishment and/or | |
| | | | maintenance of cell polarity /// | |
| | | | signal transduction /// activation of | |
| | | | adenylate cyclase activity | |
| 33440_at | 0.52335 | ZEB1 | negative regulation of transcription | 6935 |
| | | | from RNA polymerase II promoter | |
| | | | /// transcription /// regulation of | |
| | | | transcription, DNA-dependent /// | |
| | | | regulation of transcription from | |
| | | | RNA polymerase II promoter /// | |
| | | | immune response /// cell | |
| | | | proliferation /// regulation of | |
| | | | transcription | |
| 33772_at | 0.366823 | PTGER4 | immune response /// signal | 5734 |
| | | | transduction /// G-protein coupled | |
| | | | receptor protein signaling pathway | |
| | | | /// G-protein signaling, coupled to | |
| | | | cAMP nucleotide second | |
| | | | messenger /// regulation of | |
| | | | ossification | |
| 33785_at | −0.600057 | BAI2 | signal transduction /// G-protein | 576 |
| | | | coupled receptor protein signaling | |
| | | | pathway /// G-protein coupled | |
| | | | receptor protein signaling pathway | |
| | | | /// neuropeptide signaling pathway | |
| 33787_at | 0.350378 | NUAK1 | protein amino acid phosphorylation | 9891 |
| 33791_at | −1.38754 | DLEU1 | cell cycle /// negative regulation of | 10301 |
| | | | cell cycle | |
| 33882_at | −1.204573 | RAB11FIP5 | transport /// metabolic process /// | 26056 |
| | | | protein transport | |
| 33900_at | −1.640013 | FSTL3 | negative regulation of BMP | 10272 |
| | | | signaling pathway | |
| 33994_g_at | −1.586516 | MYL6 /// | muscle contraction /// skeletal | 140465 /// |
| | | MYL6B | muscle development /// muscle | 4637 |
| | | | filament sliding | |
| 34091_s_at | −0.849163 | VIM | cell motility /// intermediate | 7431 |
| | | | filament-based process | |
| 34106_at | −0.605788 | GNA12 | signal transduction /// G-protein | 2768 |
| | | | coupled receptor protein signaling | |
| | | | pathway /// G-protein coupled | |
| | | | receptor protein signaling pathway | |
| | | | /// blood coagulation | |
| 34318_at | −1.594771 | PRAF2 | transport /// protein transport /// L- | 11230 |
| | | | glutamate transport | |
| 34320_at | −0.729318 | PTRF | transcription /// transcription | 284119 |
| | | | termination /// regulation of | |
| | | | transcription, DNA-dependent /// | |
| | | | transcription initiation from RNA | |
| | | | polymerase I promoter | |
| 34375_at | −1.016405 | CCL2 | protein amino acid phosphorylation | 6347 |
| | | | /// cellular calcium ion homeostasis | |
| | | | /// anti-apoptosis /// chemotaxis /// | |
| | | | chemotaxis /// inflammatory | |
| | | | response /// immune response /// | |
| | | | humoral immune response /// cell | |
| | | | adhesion /// signal transduction /// | |
| | | | cell surface receptor linked signal | |
| | | | transduction /// G-protein coupled | |
| | | | receptor protein signaling pathway | |
| | | | /// G-protein signaling, coupled to | |
| | | | cyclic nucleotide second | |
| | | | messenger /// JAK-STAT cascade | |
| | | | /// cell-cell signaling /// organ | |
| | | | morphogenesis /// viral genome | |
| | | | replication | |
| 34795_at | −0.96244 | PLOD2 | response to hypoxia /// protein | 5352 |
| | | | modification process /// protein | |
| | | | metabolic process | |
| 34802_at | −1.080254 | COL6A2 | phosphate transport /// cell | 1292 |
| | | | adhesion /// cell-cell adhesion /// | |
| | | | extracellular matrix organization | |
| | | | and biogenesis | |
| 34811_at | 0.554813 | ATP5G3 | generation of precursor | 518 |
| | | | metabolites and energy /// | |
| | | | transport /// ion transport /// ATP | |
| | | | synthesis coupled proton transport | |
| | | | /// proton transport /// ATP | |
| | | | metabolic process | |
| 35130_at | −1.014434 | GSR | glutathione metabolic process /// | 2936 |
| | | | cell redox homeostasis | |
| 35264_at | −1.524088 | NDUFS3 | mitochondrial electron transport, | 4722 |
| | | | NADH to ubiquinone /// | |
| | | | mitochondrial electron transport, | |
| | | | NADH to ubiquinone /// protein | |
| | | | amino acid dephosphorylation /// | |
| | | | oxygen and reactive oxygen | |
| | | | species metabolic process /// | |
| | | | transport /// induction of apoptosis | |
| | | | /// dephosphorylation /// negative | |
| | | | regulation of cell growth /// | |
| | | | oxidation reduction | |
| 35309_at | −0.925755 | ST14 | proteolysis /// proteolysis | 6768 |
| 35366_at | −0.947779 | NID1 | cell adhesion /// cell-matrix | 4811 |
| | | | adhesion /// bioluminescence /// | |
| | | | protein-chromophore linkage | |
| 35729_at | −0.923216 | MYO1D | — | 4642 |
| 35751_at | −1.131622 | SDHB | tricarboxylic acid cycle /// | 6390 |
| | | | tricarboxylic acid cycle /// transport | |
| | | | /// aerobic respiration /// oxidation | |
| | | | reduction | |
| 36119_at | −0.40934 | CAV1 | inactivation of MAPK activity /// | 857 |
| | | | vasculogenesis /// response to | |
| | | | hypoxia /// negative regulation of | |
| | | | endothelial cell proliferation /// | |
| | | | triacylglycerol metabolic process /// | |
| | | | calcium ion transport /// cellular | |
| | | | calcium ion homeostasis /// | |
| | | | endocytosis /// regulation of | |
| | | | smooth muscle contraction /// | |
| | | | skeletal muscle development /// | |
| | | | protein localization /// vesicle | |
| | | | organization and biogenesis /// | |
| | | | regulation of fatty acid metabolic | |
| | | | process /// sequestering of lipid /// | |
| | | | regulation of blood coagulation /// | |
| | | | cholesterol transport /// negative | |
| | | | regulation of epithelial cell | |
| | | | differentiation /// mammary gland | |
| | | | development /// nitric oxide | |
| | | | homeostasis /// cholesterol | |
| | | | homeostasis /// cholesterol | |
| | | | homeostasis /// negative regulation | |
| | | | of MAPKKK cascade /// negative | |
| | | | regulation of nitric oxide | |
| | | | biosynthetic process /// positive | |
| | | | regulation of vasoconstriction /// | |
| | | | negative regulation of vasodilation | |
| | | | /// negative regulation of JAK- | |
| | | | STAT cascade /// positive | |
| | | | regulation of metalloenzyme | |
| | | | activity /// protein | |
| | | | homooligomerization /// membrane | |
| | | | depolarization /// regulation of | |
| | | | peptidase activity /// calcium ion | |
| | | | homeostasis /// mammary gland | |
| | | | involution | |
| 36149_at | −1.468146 | DPYSL3 | nucleobase, nucleoside, nucleotide | 1809 |
| | | | and nucleic acid metabolic process | |
| | | | /// signal transduction /// nervous | |
| | | | system development /// nervous | |
| | | | system development | |
| 36369_at | 0.937326 | PTRF | transcription /// transcription | 284119 |
| | | | termination /// regulation of | |
| | | | transcription, DNA-dependent /// | |
| | | | transcription initiation from RNA | |
| | | | polymerase I promoter | |
| 36525_at | −0.978609 | FBXL2 | protein modification process /// | 25827 |
| | | | proteolysis /// ubiquitin cycle | |
| 36550_at | −1.009504 | RIN2 | endocytosis /// signal transduction | 54453 |
| | | | /// small GTPase mediated signal | |
| | | | transduction | |
| 36577_at | −1.446371 | FERMT2 | cell adhesion /// cell adhesion /// | 10979 |
| | | | regulation of cell shape /// actin | |
| | | | cytoskeleton organization and | |
| | | | biogenesis | |
| 36638_at | 0.911469 | CTGF | cartilage condensation /// | 1490 |
| | | | ossification /// angiogenesis /// | |
| | | | regulation of cell growth /// DNA | |
| | | | replication /// cell motility /// cell | |
| | | | adhesion /// cell-matrix adhesion /// | |
| | | | integrin-mediated signaling | |
| | | | pathway /// intracellular signaling | |
| | | | cascade /// fibroblast growth factor | |
| | | | receptor signaling pathway /// | |
| | | | epidermis development /// | |
| | | | response to wounding /// cell | |
| | | | migration /// cell differentiation | |
| 36659_at | −2.116964 | COL4A2 | phosphate transport /// negative | 1284 |
| | | | regulation of angiogenesis /// | |
| | | | extracellular matrix organization | |
| | | | and biogenesis | |
| 36790_at | −1.46788 | TPM1 | cell motility /// regulation of muscle | 7168 |
| | | | contraction /// regulation of heart | |
| | | | contraction | |
| 36791_g_at | 0.634657 | TPM1 | cell motility /// regulation of muscle | 7168 |
| | | | contraction /// regulation of heart | |
| | | | contraction | |
| 36792_at | −0.771539 | TPM1 | cell motility /// regulation of muscle | 7168 |
| | | | contraction /// regulation of heart | |
| | | | contraction | |
| 36799_at | −0.81719 | FZD2 | establishment of tissue polarity /// | 2535 |
| | | | signal transduction /// signal | |
| | | | transduction /// cell surface | |
| | | | receptor linked signal transduction | |
| | | | /// G-protein coupled receptor | |
| | | | protein signaling pathway /// cell- | |
| | | | cell signaling /// multicellular | |
| | | | organismal development /// Wnt | |
| | | | receptor signaling pathway /// | |
| | | | epithelial cell differentiation | |
| 36811_at | −0.394702 | LOXL1 | protein amino acid deamination /// | 4016 |
| | | | oxidation reduction | |
| 36885_at | −2.44668 | SYK | serotonin secretion /// protein | 6850 |
| | | | complex assembly /// protein | |
| | | | amino acid phosphorylation /// | |
| | | | protein amino acid phosphorylation | |
| | | | /// leukocyte adhesion /// signal | |
| | | | transduction /// enzyme linked | |
| | | | receptor protein signaling pathway | |
| | | | /// integrin-mediated signaling | |
| | | | pathway /// intracellular signaling | |
| | | | cascade /// activation of JNK | |
| | | | activity /// cell proliferation /// organ | |
| | | | morphogenesis /// peptidyl-tyrosine | |
| | | | phosphorylation /// leukotriene | |
| | | | biosynthetic process /// neutrophil | |
| | | | chemotaxis /// positive regulation | |
| | | | of mast cell degranulation /// beta | |
| | | | selection /// positive regulation of | |
| | | | interleukin-3 biosynthetic process | |
| | | | /// positive regulation of | |
| | | | granulocyte macrophage colony- | |
| | | | stimulating factor biosynthetic | |
| | | | process /// positive regulation of B | |
| | | | cell differentiation /// positive | |
| | | | regulation of gamma-delta T cell | |
| | | | differentiation /// positive regulation | |
| | | | of alpha-beta T cell differentiation | |
| | | | /// positive regulation of alpha-beta | |
| | | | T cell proliferation /// protein amino | |
| | | | acid autophosphorylation /// | |
| | | | positive regulation of peptidyl- | |
| | | | tyrosine phosphorylation /// | |
| | | | positive regulation of calcium- | |
| | | | mediated signaling /// B cell | |
| | | | receptor signaling pathway | |
| 36952_at | −1.151528 | HADHA | lipid metabolic process /// fatty acid | 3030 |
| | | | metabolic process /// fatty acid | |
| | | | beta-oxidation /// metabolic | |
| | | | process /// response to drug | |
| 36988_at | −1.204986 | TNFAIP1 | DNA replication /// DNA replication | 7126 |
| | | | /// regulation of transcription, DNA- | |
| | | | dependent /// translation /// | |
| | | | translation /// potassium ion | |
| | | | transport /// immune response /// | |
| | | | immune response /// embryonic | |
| | | | development /// embryonic | |
| | | | development | |
| 37032_at | −1.245426 | NNMT | — | 4837 |
| 37322_s_at | −2.270441 | HPGD | lipid metabolic process /// fatty acid | 3248 |
| | | | metabolic process /// prostaglandin | |
| | | | metabolic process /// prostaglandin | |
| | | | metabolic process /// transforming | |
| | | | growth factor beta receptor | |
| | | | signaling pathway /// female | |
| | | | pregnancy /// parturition /// | |
| | | | metabolic process /// lipoxygenase | |
| | | | pathway /// negative regulation of | |
| | | | cell cycle | |
| 37408_at | −1.250313 | MRC2 | endocytosis | 9902 |
| 37486_f_at | −0.773001 | MEIS3P1 | regulation of transcription, DNA- | 4213 |
| | | | dependent /// regulation of | |
| | | | transcription | |
| 37599_at | −0.440975 | AOX1 | oxygen and reactive oxygen | 316 |
| | | | species metabolic process /// | |
| | | | inflammatory response /// oxidation | |
| | | | reduction | |
| 376_at | 0.358759 | SEMA3C | immune response /// | 10512 |
| | | | transmembrane receptor protein | |
| | | | tyrosine kinase signaling pathway | |
| | | | /// multicellular organismal | |
| | | | development /// response to drug | |
| 377_g_at | 1.565062 | SEMA3C | immune response /// | 10512 |
| | | | transmembrane receptor protein | |
| | | | tyrosine kinase signaling pathway | |
| | | | /// multicellular organismal | |
| | | | development /// response to drug | |
| 38113_at | −0.971934 | SYNE1 | nuclear organization and | 23345 |
| | | | biogenesis /// Golgi organization | |
| | | | and biogenesis /// keratinization /// | |
| | | | muscle cell differentiation | |
| 38125_at | −1.45776 | SERPINE1 | blood coagulation /// fibrinolysis /// | 5054 |
| | | | regulation of angiogenesis | |
| 38299_at | −0.839698 | IL6 | neutrophil apoptosis /// neutrophil | 3569 |
| | | | apoptosis /// acute-phase response | |
| | | | /// inflammatory response /// | |
| | | | immune response /// humoral | |
| | | | immune response /// cell surface | |
| | | | receptor linked signal transduction | |
| | | | /// cell-cell signaling /// cell-cell | |
| | | | signaling /// positive regulation of | |
| | | | cell proliferation /// negative | |
| | | | regulation of cell proliferation /// | |
| | | | positive regulation of peptidyl- | |
| | | | serine phosphorylation /// defense | |
| | | | response to protozoan /// | |
| | | | regulation of apoptosis /// negative | |
| | | | regulation of apoptosis /// positive | |
| | | | regulation of MAPKKK cascade /// | |
| | | | negative regulation of chemokine | |
| | | | biosynthetic process /// negative | |
| | | | regulation of chemokine | |
| | | | biosynthetic process /// positive | |
| | | | regulation of T-helper 2 cell | |
| | | | differentiation /// positive regulation | |
| | | | of translation /// positive regulation | |
| | | | of transcription, DNA-dependent /// | |
| | | | positive regulation of transcription | |
| | | | from RNA polymerase II promoter | |
| | | | /// negative regulation of hormone | |
| | | | secretion /// positive regulation of | |
| | | | peptidyl-tyrosine phosphorylation | |
| | | | /// response to glucocorticoid | |
| | | | stimulus | |
| 38338_at | −0.844895 | RRAS | small GTPase mediated signal | 6237 |
| | | | transduction /// Ras protein signal | |
| | | | transduction /// negative regulation | |
| | | | of cell migration | |
| 38394_at | −1.836007 | GPD1L | carbohydrate metabolic process /// | 23171 |
| | | | glycerol-3-phosphate metabolic | |
| | | | process /// metabolic process /// | |
| | | | glycerol-3-phosphate catabolic | |
| | | | process | |
| 38396_at | −0.915548 | MAP1B | microtubule bundle formation /// | 4131 |
| | | | negative regulation of microtubule | |
| | | | depolymerization /// dendrite | |
| | | | development | |
| 38433_at | 0.24027 | AXL | protein amino acid phosphorylation | 558 |
| | | | /// signal transduction | |
| 38449_at | −2.322063 | WDR23 | — | 80344 |
| 38482_at | −2.584731 | CLDN7 | calcium-independent cell-cell | 1366 |
| | | | adhesion | |
| 38488_s_at | −1.103501 | IL15 | immune response /// immune | 3600 |
| | | | response /// signal transduction /// | |
| | | | cell-cell signaling /// positive | |
| | | | regulation of cell proliferation | |
| 38631_at | 0.472464 | TNFAIP2 | angiogenesis /// multicellular | 7127 |
| | | | organismal development /// cell | |
| | | | differentiation | |
| 38772_at | −1.647628 | CYR61 | regulation of cell growth /// | 3491 |
| | | | chemotaxis /// cell adhesion /// cell | |
| | | | proliferation /// anatomical | |
| | | | structure morphogenesis | |
| 38775_at | −0.628315 | LRP1 /// | lipid metabolic process /// | 100134190 |
| | | LOC100134190 | endocytosis /// multicellular | /// 4035 |
| | | | organismal development /// cell | |
| | | | proliferation | |
| 38842_at | 0.382258 | AMOTL2 | — | 51421 |
| 38921_at | −0.553897 | PDE1B | apoptosis /// signal transduction | 5153 |
| 39100_at | 0.440558 | SPOCK1 | cell motility /// cell adhesion /// | 6695 |
| | | | multicellular organismal | |
| | | | development /// nervous system | |
| | | | development /// cell proliferation | |
| 39254_at | −1.452007 | RAI14 | — | 26064 |
| 39277_at | 0.600055 | OSMR | cell surface receptor linked signal | 9180 |
| | | | transduction /// cell proliferation | |
| 39327_at | −0.423697 | PXDN | immune response /// response to | 7837 |
| | | | oxidative stress /// hydrogen | |
| | | | peroxide catabolic process /// | |
| | | | oxidation reduction | |
| 39333_at | −3.12657 | COL4A1 | phosphate transport | 1282 |
| 39409_at | 0.638946 | C1R | proteolysis /// immune response /// | 715 |
| | | | immune response /// complement | |
| | | | activation, classical pathway /// | |
| | | | innate immune response | |
| 39614_at | −0.683932 | KIAA0802 | — | 23255 |
| 39710_at | −0.703314 | C5orf13 | regulation of transforming growth | 9315 |
| | | | factor beta receptor signaling | |
| | | | pathway | |
| 39867_at | −1.328942 | TUFM | translation /// translational | 7284 |
| | | | elongation /// translational | |
| | | | elongation | |
| 39901_at | 0.370034 | EDIL3 | cell adhesion /// multicellular | 10085 |
| | | | organismal development | |
| 40023_at | 0.527535 | BDNF | nervous system development | 627 |
| 40078_at | 0.311987 | PRSS23 | proteolysis | 11098 |
| 40096_at | −2.35259 | ATP5A1 | negative regulation of endothelial | 498 |
| | | | cell proliferation /// ATP | |
| | | | biosynthetic process /// transport /// | |
| | | | ion transport /// ATP synthesis | |
| | | | coupled proton transport /// proton | |
| | | | transport | |
| 40171_at | −2.001626 | FRAT2 | multicellular organismal | 23401 |
| | | | development /// cell proliferation /// | |
| | | | Wnt receptor signaling pathway | |
| 40341_at | −0.929303 | TMEM186 | — | 25880 |
| 40497_at | −1.09089 | TUSC4 | cell cycle /// negative regulation of | 10641 |
| | | | cell cycle | |
| 40564_at | −1.429238 | NUP50 | transport /// protein transport /// | 10762 |
| | | | intracellular transport /// mRNA | |
| | | | transport /// intracellular protein | |
| | | | transport across a membrane | |
| 40567_at | −0.263158 | TUBA1A | microtubule-based process /// | 7846 |
| | | | microtubule-based movement /// | |
| | | | protein polymerization | |
| 40642_at | −2.210099 | NFIB | DNA replication /// transcription /// | 4781 |
| | | | regulation of transcription, DNA- | |
| | | | dependent | |
| 40692_at | −0.519972 | TLE4 | transcription /// regulation of | 7091 |
| | | | transcription, DNA-dependent /// | |
| | | | Wnt receptor signaling pathway /// | |
| | | | regulation of transcription | |
| 40781_at | 0.252463 | AKT3 | protein amino acid phosphorylation | 10000 |
| | | | /// protein amino acid | |
| | | | phosphorylation /// signal | |
| | | | transduction | |
| 40936_at | −1.518856 | CRIM1 | regulation of cell growth /// | 51232 |
| | | | proteolysis /// nervous system | |
| | | | development | |
| 41197_at | 1.636782 | RAD23A | DNA repair /// nucleotide-excision | 5886 |
| | | | repair /// nucleotide-excision repair | |
| | | | /// protein modification process /// | |
| | | | response to DNA damage stimulus | |
| | | | /// proteasomal ubiquitin- | |
| | | | dependent protein catabolic | |
| | | | process | |
| 41223_at | 0.425264 | COX5A | oxidation reduction | 9377 |
| 41236_at | 0.532242 | SMCR7L | — | 54471 |
| 41273_at | −0.865492 | MXRA7 | — | 439921 |
| 41295_at | −1.468337 | STARD7 | — | 56910 |
| 41354_at | 0.22724 | STC1 | cellular calcium ion homeostasis /// | 6781 |
| | | | cell surface receptor linked signal | |
| | | | transduction /// cell-cell signaling /// | |
| | | | response to nutrient | |
| 41478_at | 0.785668 | TTC28 | — | 23331 |
| 41544_at | 0.959715 | PLK2 | mitotic cell cycle /// protein amino | 10769 |
| | | | acid phosphorylation /// positive | |
| | | | regulation of I-kappaB kinase/NF- | |
| | | | kappaB cascade | |
| 41667_s_at | −1.111511 | TGDS | metabolic process /// cellular | 23483 |
| | | | metabolic process | |
| 41738_at | −1.668471 | CALD1 | cell motility /// muscle contraction | 800 |
| 41744_at | −1.398598 | OPTN | protein targeting to Golgi /// Golgi | 10133 |
| | | | organization and biogenesis /// | |
| | | | signal transduction /// cell death /// | |
| | | | Golgi to plasma membrane protein | |
| | | | transport | |
| 41745_at | −0.837378 | IFITM3 | immune response /// response to | 10410 |
| | | | biotic stimulus | |
| 41872_at | −1.128956 | DFNA5 | sensory perception of sound /// | 1687 |
| | | | sensory perception of sound /// | |
| | | | inner ear receptor cell | |
| | | | differentiation | |
| 424_s_at | −1.471912 | FGFR1 | MAPKKK cascade /// skeletal | 2260 |
| | | | development /// protein amino acid | |
| | | | phosphorylation /// protein amino | |
| | | | acid phosphorylation /// fibroblast | |
| | | | growth factor receptor signaling | |
| | | | pathway /// fibroblast growth factor | |
| | | | receptor signaling pathway /// cell | |
| | | | growth | |
| 465_at | −1.303867 | HTATIP | regulation of cell growth /// double- | 10524 |
| | | | strand break repair /// chromatin | |
| | | | assembly or disassembly /// | |
| | | | transcription /// regulation of | |
| | | | transcription, DNA-dependent /// | |
| | | | transcription from RNA polymerase | |
| | | | II promoter /// chromatin | |
| | | | modification /// histone acetylation | |
| | | | /// androgen receptor signaling | |
| | | | pathway /// positive regulation of | |
| | | | transcription, DNA-dependent | |
| 548_s_at | −2.690624 | SYK | serotonin secretion /// protein | 6850 |
| | | | complex assembly /// protein | |
| | | | amino acid phosphorylation /// | |
| | | | protein amino acid phosphorylation | |
| | | | /// leukocyte adhesion /// signal | |
| | | | transduction /// enzyme linked | |
| | | | receptor protein signaling pathway | |
| | | | /// integrin-mediated signaling | |
| | | | pathway /// intracellular signaling | |
| | | | cascade /// activation of JNK | |
| | | | activity /// cell proliferation /// organ | |
| | | | morphogenesis /// peptidyl-tyrosine | |
| | | | phosphorylation /// leukotriene | |
| | | | biosynthetic process /// neutrophil | |
| | | | chemotaxis /// positive regulation | |
| | | | of mast cell degranulation /// beta | |
| | | | selection /// positive regulation of | |
| | | | interleukin-3 biosynthetic process | |
| | | | /// positive regulation of | |
| | | | granulocyte macrophage colony- | |
| | | | stimulating factor biosynthetic | |
| | | | process /// positive regulation of B | |
| | | | cell differentiation /// positive | |
| | | | regulation of gamma-delta T cell | |
| | | | differentiation /// positive regulation | |
| | | | of alpha-beta T cell differentiation | |
| | | | /// positive regulation of alpha-beta | |
| | | | T cell proliferation /// protein amino | |
| | | | acid autophosphorylation /// | |
| | | | positive regulation of peptidyl- | |
| | | | tyrosine phosphorylation /// | |
| | | | positive regulation of calcium- | |
| | | | mediated signaling /// B cell | |
| | | | receptor signaling pathway | |
| 581_at | −1.565877 | LAMB1 | cell adhesion /// cell adhesion /// | 3912 |
| | | | positive regulation of cell migration | |
| | | | /// neurite development /// | |
| | | | odontogenesis /// positive | |
| | | | regulation of epithelial cell | |
| | | | proliferation | |
| 628_at | −0.861772 | FZD2 | establishment of tissue polarity /// | 2535 |
| | | | signal transduction /// signal | |
| | | | transduction /// cell surface | |
| | | | receptor linked signal transduction | |
| | | | /// G-protein coupled receptor | |
| | | | protein signaling pathway /// cell- | |
| | | | cell signaling /// multicellular | |
| | | | organismal development /// Wnt | |
| | | | receptor signaling pathway /// | |
| | | | epithelial cell differentiation | |
| 672_at | −1.059245 | SERPINE1 | blood coagulation /// fibrinolysis /// | 5054 |
| | | | regulation of angiogenesis | |
| 867_s_at | 0.423802 | THBS1 | cell motility /// cell adhesion /// | 7057 |
| | | | multicellular organismal | |
| | | | development /// nervous system | |
| | | | development /// blood coagulation | |
| 875_g_at | −0.889978 | CCL2 | protein amino acid phosphorylation | 6347 |
| | | | /// cellular calcium ion homeostasis | |
| | | | /// anti-apoptosis /// chemotaxis /// | |
| | | | chemotaxis /// inflammatory | |
| | | | response /// immune response /// | |
| | | | humoral immune response /// cell | |
| | | | adhesion /// signal transduction /// | |
| | | | cell surface receptor linked signal | |
| | | | transduction /// G-protein coupled | |
| | | | receptor protein signaling pathway | |
| | | | /// G-protein signaling, coupled to | |
| | | | cyclic nucleotide second | |
| | | | messenger /// JAK-STAT cascade | |
| | | | /// cell-cell signaling /// organ | |
| | | | morphogenesis /// viral genome | |
| | | | replication | |
| 884_at | −1.840205 | ITGA3 | cell adhesion /// cell-matrix | 3675 |
| | | | adhesion /// integrin-mediated | |
| | | | signaling pathway | |
| 885_g_at | −2.1017 | ITGA3 | cell adhesion /// cell-matrix | 3675 |
| | | | adhesion /// integrin-mediated | |
| | | | signaling pathway | |
| 890_at | −1.809728 | UBE2A | DNA repair /// postreplication | 7319 |
| | | | repair /// ubiquitin-dependent | |
| | | | protein catabolic process /// | |
| | | | ubiquitin cycle /// response to DNA | |
| | | | damage stimulus /// post- | |
| | | | translational protein modification /// | |
| | | | regulation of protein metabolic | |
| | | | process | |
| 919_at | −0.953292 | — | — | — |

TABLE 8
PI3 Kinase inhibitor responsivity predictor set

| Gene Symbol | Affymetrix Probe ID | Gene Title |
|---|---|---|
| RFC2 | 1053_at | replication factor C (Activator 1) 2, 40 kDa |
| KIAA0153 | 1552257_a_at | KIAA0153 protein |
| EXOSC6 | 1553947_at | exosome component 6 |
| RHOB | 1553962_s_at | ras homolog gene family, member B |
| MAD2L1 | 1554768_a_at | MAD2 mitotic arrest deficient-like 1 (yeast) |
| RBM15 | 1555762_s_at | RNA binding motif protein 15 |
| SPEN | 1556059_s_at | spen homolog, transcriptional regulator |
| | | (Drosophilia) |
| C6orf150 | 1559051_s_at | chromosome 6 reading frame 150 |
| HSPA1A | 200799_at | heat shock 70 kDa protein 1A |
| HSPA1A///HSPA1B | 200800_s_at | heat shock 70 kDa protein 1A///heat shock |
| | | 70 kDa protein 1B |
| NOL5A | 200875_s_at | nucleolar protein 5A (56 kDa with KKE/D |
| | | repeat) |
| CSE1L | 201112_s_at | CSE1 chromosome segregation 1-like (yeast) |
| PCNA | 201202_at | proliferating cell nuclear antigen |
| JUN | 201464_x_at | v-jun sarcoma virus 17 oncogene homolog |
| | | (avian) |
| JUN | 201465_s_at | v-jun sarcoma virus 17 oncogene homolog |
| | | (avian) |
| JUN | 201466_s_at | v-jun sarcoma virus 17 oncogene homolog |
| | | (avian) |
| JUNB | 201473_at | jun B proto-oncogene |
| MCM3 | 201555_at | MCM3 minichromosome maintenance deficient |
| | | 3 (S. cerevisiae) |
| EGR1 | 201693_s_at | early growth response 1 |
| DNMT1 | 201697_s_at | DNA (cytosine-5-)-methyltransferase 1 |
| MCM5 | 201755_at | MCM5 minichromosome maintenance deficient |
| | | 5, cell division cycle 46 (S. cerevisiae) |
| RRM2 | 201890_at | ribonucleotide reductase M2 polypeptide |
| MCM6 | 201930_at | MCM6 minichromosome maintenance deficient |
| | | 6, (MISS homolog, S. pombe) (S. cerevisiae) |
| NASP | 201970_s_at | nuclear autoantigenic sperm protein (histone- |
| | | binding) |
| SPEN | 201997_s_at | spen homolog, transcriptional regulator |
| | | (Drosophilia) |
| IER2 | 202081_at | immediate early response 2 |
| MCM2 | 202107_s_at | MCM2 minichromosome maintenance deficient |
| | | 2, mitotin (S. cerevisiae) |
| MTHFD1 | 202309_at | methylenetetrahydrofolate dehydrogenase |
| | | (NADP+dependant) 1, |
| | | methylenetetrahydrofolate cyclohydrolase, |
| | | formyltetrahydrofolate synthetase |
| UNG | 202330_s_at | uracil-DNA glycosylase |
| HSPA1B | 202581_at | heat shock 70 kDa protein 1B |
| MSH6 | 202911_at | mutS homolog 6 (E. coli) |
| SSX2IP | 203017_s_at | synovial sarcoma, X breakpoint 2 interacting |
| | | protein |
| RNASEH2A | 203022_at | ribonuclease H2, large subunit |
| PEX5 | 203244_at | peroxisomal biogenesis factor 5 |
| LMNB1 | 203276_at | lamin B1 |
| POLD1 | 203422_at | polymerase (DNA directed), delta 1, catalytic |
| | | subunit 125 kDa |
| CDC6 | 203968_s_at | CDC6 cell division cycle 6 homolog (S. cerevisiae) |
| ZWINT | 204026_s_at | ZW10 interactor |
| CDC45L | 204126_s_at | CDC45 cell division cycle 45-like (S. cerevisiae) |
| RFC3 | 204128_s_at | replication factor C (activator 1) 3, 38 kDa |
| POLA2 | 204441_s_at | polymerase (DNA directed), alpha 2 (70 kD |
| | | subunit |
| CDC7 | 204510_at | CDC7 cell division cycle 7 (S. cerevisiae) |
| DIPA | 204610_s_at | hepatitis delta antigen-interacting protein A |
| ACD | 204617_s_at | adrenocortical dysplasia homolog (mouse) |
| CDC25A | 204695_at | cell division cycle 25A |
| FEN1 | 204767_s_at | flap structure-specific endonuclease 1 |
| FEN1 | 204768_s_at | flap structure-specific endonuclease 1 |
| MYB | 204798_at | v-myb myeloblastosis viral oncogene homolog |
| | | (avian) |
| TOP3A | 204946_s_at | topoisomerase (DNA) III alpha |
| DDX10 | 204977_at | DEAD (Asp-Glu-Ala-Asp) box polypeptide 10 |
| RAD51 | 205024_s_at | RAD51 homolog (RecA homolog, E. coli) S. cerevisiae) |
| CCNE2 | 205034_at | cyclin E2 |
| PRIM1 | 205053_at | primase, polypeptide 1, 49 kDa |
| BARD1 | 205345_at | BRCA1 associated RING domain 1 |
| CHEK1 | 205393_s_at | CHK1 checkpoint homolog (S. pombe) |
| H2AFX | 205436_s_at | H2A histone family, member X |
| FLJI2973 | 205519_at | hypothetical protein FLJI2973 |
| GEMIN4 | 205527_s_at | gem (nuclear organelle) associated protein 4 |
| SLBP | 206052_s_at | stem-loop (histone) binding protein |
| KIAA0186 | 206102_at | KIAA0186 gene product |
| AKR7A3 | 206469_x_at | aldo-keto reductase family 7, member A3 |
| | | (aflatoxin aldehyde reductase) |
| TLE3 | 206472_s_at | transducin-like enhancer of split 3 (E(spl) |
| | | homolog, Drosophilia) |
| GADD45B | 207574_s_at | growth arrest and DNA-damage-inducible, beta |
| PRPS1 | 207447_s_at | phosphoribosyl pyrophosphate synthetase 1 |
| BRD2 | 208685_x_at | bromodomain containing 2 |
| MCM7 | 208795_s_at | MCM7 minichromosome maintenance deficient |
| | | 7 (S. cerevisiae) |
| ID1 | 208937_s_at | inhibitor of DNA binding 1, dominant negative |
| | | helix-loop-helix protein |
| GADD45B | 209304_x_at | growth arrest and DNA-damage-inducible, beta |
| GADD45B | 209305_s_at | growth arrest and DNA-damage-inducible, beta |
| POLR1C | 209317_at | polymerase (RNA) I polypeptide C, 30 kDa |
| PRKRIR | 209323_at | protein-kinase, interferon-inducible double |
| | | stranded RNA dependent inhibitor, repressor of |
| | | (P58 repressor) |
| MSH2 | 209421_at | mutS homolog 2, colon cancer, nonpolyposis |
| | | type 1 (E. coli) |
| PPAT | 209433_s_at | phosphoribosyl pyrophosphate |
| | | amidotransferase |
| PPAT | 209434_s_at | phosphoribosyl pyrophosphate |
| | | amidotransferase |
| PRPS1 | 209440_at | phosphoribosyl pyrophosphate synthetase 1 |
| RPA3 | 209507_at | replication protein A3, 14 kDa |
| EED | 209572_s_at | embryonic ectoderm development |
| GAS2L1 | 209729_at | growth arrest-specific 2 like 1 |
| RPM2 | 209773_s_at | ribonucleotide reductase M2 polypeptide |
| SLC19A1 | 209777_s_at | solute carrier family 19 (folate transporter), |
| | | member 1 |
| CDT1 | 209832_s_at | DNA replication factor |
| SHMT1 | 209980_s_at | serine hydroxymethyltranse 1 (soluable) |
| TAF5 | 210053_at | TAF5 RNA polymerase II, TATA box binding |
| | | protein (TBP)-associated factor, 100 kDa |
| MCM7 | 210983_s_at | MCM7 minichromosome maintenance deficient |
| | | 7 (S. cerevisiae) |
| MSH6 | 211450_s_at | mutS homolog 6 (E. coli) |
| CCNE2 | 211814_s_at | cyclin E2 |
| RHOB | 212099_at | ras homolog gene family, member B |
| MCM4 | 212141_at | MCM4 minichromosome maintenance deficient |
| | | 4 (S. cerevisiae) |
| MCM4 | 212142_at | MCM4 minichromosome maintenance deficient |
| | | 4 (S. cerevisiae) |
| KCTD12 | 212188_at | potassium channel tetramerisation domain |
| | | containing 12/// potassium channel |
| | | tetramerisation domain containing 12 |
| KCTD12 | 212192_at | potassium channel tetramerisation domain |
| | | containing 12 |
| MAC30 | 212281_s_at | hypothetical protein MAC30 |
| POLD3 | 212836_at | polymerase (DNA-directed), delta 3, accessory |
| | | subunit |
| KIAA0406 | 212898_at | KIAA0406 gene product |
| FLJ10719 | 213007_at | hypothetical protein FLJ10719 |
| ITPKC | 213076_at | inositol 1,4,5-triphosphate 3-kinase C |
| ZNF473 | 213124_at | zinc finger protein |
| — | 213281_at | — |
| CCNE1 | 213523_at | cyclin E1 |
| GADD45B | 213560_at | Growth arrest and DNA-damage-inductible, |
| | | beta |
| GAL | 214240_at | galanin |
| BRD2 | 214911_s_at | bromodomain containing 2 |
| UMPS | 215165_x_at | uridine monophosphate synthetase (orotate |
| | | phosphoribosyl transferase and orotidine-5′- |
| | | decarboxylase) |
| MCM5 | 216237_s_at | MCM5 minichromosome maintenance deficient |
| | | 5, cell division cycle 46 (S. cerevisiae) |
| LAMNB2 | 216952_s_at | lamin B2 |
| GEMIN4 | 217099_s_at | gem (nuclear organelle) associated protein 4 |
| SUPT16H | 217815_at | suppressor of Ty 16 homolog (S. cerevisae) |
| GMNN | 218350_s_at | geminin, DNA replication inhibitor |
| RAMP | 218585_s_at | RA-regulated nuclear matrix-associated protein |
| SLC25A15 | 218653_at | solute carrier family 25 (mitochondrial carrier; |
| | | ornithine transporter) member 15 |
| FLJ13912 | 218719_s_at | hypothetical protein FLJ13912 |
| ATAD2 | 218782_s_at | ATpase family, AAA domain containing 2 |
| C10orf117 | 21889_at | chromosome 10 open reading frame 117 |
| MGC10993 | 218897_at | hypothetical protein MGC10993 |
| C21orf45 | 219004_s_at | chromosome 21 open reading frame 45 |
| RPP25 | 219143_s_at | ribonuclease P 25 kDa subunit |
| FJL20516 | 219258_at | timeless-interacting protein |
| MGC4504 | 219270_at | hypothetical protein MGC4504 |
| RBM15 | 219286_s_at | RNA binding motif protein 15 |
| FLJ11078 | 219254_at | hypothetical protein FLJ11078 |
| DCLRE1B | 219490_s_at | DNA cross-link repair 1B (PSO2 homolog, S. cerevisiae) |
| FLJ34077 | 219731_at | weakly similar to zinc finger protein |
| FLJ20257 | 219798_s_at | hypothetical protein FLJ20257 |
| MCM10 | 220651_s_at | MCM10 minichromosome maintenance |
| | | deficient 10 (S. cerevisiae) |
| TBRG4 | 220789_s_at | transforming growth factor beta regulator 4 |
| Pfs2 | 221521_s_at | DNA replication complex GINS protein PSF2 |
| LEF1 | 221558_s_at | lymphoid enhancer-binding factor 1 |
| ZNF45 | 222028_at | zinc finger protein 45 |
| MCM4 | 222036_s_at | MCM4 minichromosome maintenance deficient |
| | | 4 (S. cerevisiae) |
| MCM4 | 222037_at | MCM4 minichromosome maintenance deficient |
| | | 4 (S. cerevisiae) |
| CASP8AP2 | 22201_s_at | CASP8 associated protein 2 |
| MGC4692 | 222622_at | Hypothetical protein MGC4692 |
| RAMP | 222680_s_at | RA-regulated nuclear matrix-associated protein |
| FIGNL1 | 222843_at | fidgetin-like 1 |
| SLC25A19 | 223222_at | solute carrier family 25 (mitochondrial |
| | | deoxynucleotide carrier) member 19 |
| UBE2T | 223229_at | ubiquitin-conjugating enzyme E2T (putative) |
| TCF19 | 223274_at | transcription factor 19 (SC1) |
| PDXP | 223290_at | pyridoxal (pyridoxine, vitamin B6) phosphatase |
| POLR1B | 223403_s_at | polymerase (RNA) I polypeptide B, 128 kDa |
| ANKRD32 | 223542_at | ankyrin repeat domain 32 |
| IL17RB | 224361_s_at | interleukin 17 receptor B/// interleukin 17 |
| | | receptor B |
| CDCA7 | 224428_s_at | cell division cycle associated 7 /// cell division |
| | | cycle associated 7 |
| MGC13096 | 224467_s_at | hypothetical protein MGC13096 /// hypothetical |
| | | protein MGC13096 |
| CDCA5 | 224752_at | cell division cycle associated 5 |
| TMEM18 | 225489_at | transmembrane protein 18 |
| MGC20419 | 225641_at | hypothetical protein BC012173 |
| UHRF1 | 225655_at | ubiquitin-like, containing PHD and RING finger |
| | | domains, 1 |
| — | 225716_at | Full-length cDNA clone CS0DK008Y109 of |
| | | HeLa cells Cot 25-normalized of Homo sapiens |
| | | (human) |
| MGC23280 | 226121_at | hypothetical protein MGC23280 |
| C13orf8 | 226194_at | chromosome 13 open reading frame 8 |
| — | 226832_at | Hypothetical LOC389188 |
| EGR1 | 227404_s_at | Early growth response 1 |
| ZMYND19 | 227477_at | zinc finger, MYND domain containing 19 |
| BARD1 | 227545_at | BRCA1 associated RING domain 1 |
| KIAA1393 | 227653_at | KIAA1393 |
| GPR27 | 227769_at | G protein-coupled receptor 27 |
| RP13-15M17.2 | 228671_at | Novel protein |
| IL17D | 228977_at | Interleukin 17D |
| JPH1 | 229139_at | junctophilin 1 |
| ZNF367 | 229551_x_at | zinc finger protein 367 |
| MGC35521 | 235431_s_at | pellino 3 alpha |
| — | 239312_at | Transcribed locus |
| CSPG5 | 39966_at | chondroitin sulfate proteoglycan 5 (neuroglycan |
| | | C) |

TABLE 9
5-Flourouracil cell lines

| | | Resistant or Sensitive |
|---|---|---|
| 5-FU | Tissue of Origin | (Res or Sen) |
| MCF7 | Breast | Sen |
| COLO 205 | Colon | Sen |
| HCT-116 | Colon | Sen |
| NCI-H460 | Non-Small Cell Lung | Sen |
| LOX IMVI | Melanoma | Sen |
| SK-MEL-5 | Melanoma | Sen |
| A498 | Renal | Sen |
| UO-31 | Renal | Sen |
| NCI/ADR-RES | Ovarian | Res |
| MDA-MB-435 | Melanoma | Res |
| SW-620 | Colon | Res |
| EKVX | Non-Small Cell Lung | Res |
| M14 | Melanoma | Res |
| SN12C | Renal | Res |
| OVCAR-8 | Ovarian | Res |

TABLE 10
Adriamycin cell lines

| | | Resistant or Sensitive |
|---|---|---|
| Adriamycin | Tissue of Origin | (Res or Sen) |
| SF-539 | CNS | Sen |
| SNB-75 | CNS | Sen |
| MDA-MB-435 | Melanoma | Sen |
| NCI-H23 | Non-Small Cell Lung | Sen |
| M14 | Melanoma | Sen |
| MALME-3M | Melanoma | Sen |
| SK-MEL-2 | Melanoma | Sen |
| SK-MEL-28 | Melanoma | Sen |
| SK-MEL-5 | Melanoma | Sen |
| UACC-62 | Melanoma | Sen |
| NCI/ADR-RES | Ovarian | Res |
| HCT-15 | Colon | Res |
| HT29 | Colon | Res |
| EKVX | Non-Small Cell Lung | Res |
| NCI-H322M | Non-Small Cell Lung | Res |
| IGROV1 | Ovarian | Res |
| OVCAR-3 | Ovarian | Res |
| OVCAR-4 | Ovarian | Res |
| OVCAR-5 | Ovarian | Res |
| OVCAR-8 | Ovarian | Res |
| SK-OV-3 | Ovarian | Res |
| CAKI-1 | Renal | Res |

TABLE 11
Cytotoxan cell lines

| | | Resistant or Sensitive |
|---|---|---|
| Cytotoxan | Tissue of Origin | (Res or Sen) |
| K-562 | Leukemia | Sen |
| MOLT-4 | Leukemia | Sen |
| HL-60(TB) | Leukemia | Sen |
| MCF7 | Breast | Sen |
| HCC-2998 | Colon | Sen |
| HCT-116 | Colon | Sen |
| NCI-H460 | Non-Small Cell Lung | Sen |
| TK-10 | Renal | Sen |
| SNB-19 | CNS | Res |
| HS 578T | Breast | Res |
| MDA-MB-231/A | Breast | Res |
| MDA-MB-435 | Melanoma | Res |
| NCI-H226 | Non-Small Cell Lung | Res |
| M14 | Melanoma | Res |
| MALME-3M | Melanoma | Res |
| SK-MEL-2 | Melanoma | Res |

TABLE 12
Taxotere (docetaxel) cell lines

| | | Resistant or Sensitive |
|---|---|---|
| Taxotere | Tissue of Origin | (Res or Sen) |
| EKVX | Non-Small Cell Lung | Res |
| IGROV1 | Ovarian | Res |
| OVCAR-4 | Ovarian | Res |
| 786-0 | Renal | Res |
| CAKI-1 | Renal | Res |
| SN12C | Renal | Res |
| TK-10 | Renal | Res |
| HL-60(TB) | Leukemia | Sen |
| SF-539 | CNS | Sen |
| HT29 | Colon | Sen |
| HOP-62 | Non-Small Cell Lung | Sen |
| SK-MEL-2 | Melanoma | Sen |
| SK-MEL-5 | Melanoma | Sen |
| NCI-H522 | Non-Small Cell Lung | Sen |

TABLE 13
Etoposide cell lines

| | | Resistant or Sensitive (Res |
|---|---|---|
| Etoposide | Tissue of Origin | or Sen) |
| SF-539 | CNS | Sen |
| BT-549 | Breast | Sen |
| MDA-MB-231 | Breast | Sen |
| HCC-2998 | Colon | Sen |
| HOP-62 | Non-Small Cell Lung | Sen |
| NCI-H226 | Non-Small Cell Lung | Sen |
| M14 | Melanoma | Sen |
| PC-3 | Prostate | Sen |
| 786-0 | Renal | Sen |
| MCF7 | Breast | Res |
| NCI/ADR-RES | Ovarian | Res |
| HCT-15 | Colon | Res |
| SW-620 | Colon | Res |
| NCI-H322M | Non-Small Cell Lung | Res |
| UACC-257 | Melanoma | Res |
| OVCAR-4 | Ovarian | Res |
| OVCAR-5 | Ovarian | Res |

TABLE 14
Taxol cell lines

| | | Resistant or Sensitive |
|---|---|---|
| Taxol | Tissue of Origin | (Res or Sen) |
| SF-295 | CNS | Sen |
| SF-539 | CNS | Sen |
| HS 578T | Breast | Sen |
| MDA-MB-435 | Melanoma | Sen |
| COLO 205 | Colon | Sen |
| HCC-2998 | Colon | Sen |
| HT29 | Colon | Sen |
| OVCAR-3 | Ovarian | Sen |
| NCI-H522 | Non-Small Cell Lung | Sen |
| CCRF-CEM | Leukemia | Res |
| SW-620 | Colon | Res |
| A549/ATCC | Non-Small Cell Lung | Res |
| EKVX | Non-Small Cell Lung | Res |
| MALME-3M | Melanoma | Res |
| SK-MEL-28 | Melanoma | Res |
| OVCAR-8 | Ovarian | Res |
| 786-0 | Renal | Res |

TABLE 15
Topotecan cell lines

| | | Resistant or Sensitive |
|---|---|---|
| Topotecan | Tissue of Origin | (Res or Sen) |
| SF-539 | CNS | Sen |
| SNB-75 | CNS | Sen |
| U251 | CNS | Sen |
| HS 578T | Breast | Sen |
| HOP-62 | Non-Small Cell Lung | Sen |
| NCI-H226 | Non-Small Cell Lung | Sen |
| NCI-H23 | Non-Small Cell Lung | Sen |
| LOXIMVI | Melanoma | Sen |
| OVCAR-8 | Ovarian | Sen |
| A498 | Renal | Sen |
| ACHN | Renal | Sen |
| CAKI-1 | Renal | Sen |
| UO-31 | Renal | Sen |
| K-562 | Leukemia | Res |
| RPMI-8226 | Leukemia | Res |
| MDA-MB-435 | Melanoma | Res |
| MDA-MB-231 | Breast | Res |
| HCC-2998 | Colon | Res |
| HCT-116 | Colon | Res |
| HCT-15 | Colon | Res |
| NCI-H322M | Non-Small Cell Lung | Res |
| SK-MEL-28 | Melanoma | Res |
| COLO 205 | Colon | Res |

TABLE 16
Validation of predictor sets in cell line and patient data sets

| | | Genomic-based |
|---|---|---|
| | Actual Overall | Prediction of Response |
| Tumor Data set/Response | Response | (i.e. PPV for Response) |
| Breast Tumor Data | | |
| MDACC | 13/51 (25.4%) | 11/13 (85.7%) |
| Adjuvant | 33/45 (66.6%) | 28/31 (90.3%) |
| Neoadjuvant Docetaxel | 13/24 (54.1%) | 11/13 (85.7%) |
| Ovarian | | |
| Topotecan | 20/48 (41.6%) | 17/22 (77.3%) |
| Paclitaxel | 20/35 (57.1%) | 20/28 (71.5%) |
| Docetaxel | 7/14 (50%) | 6/7 (85.7%) |
| Adriamycin (Evans et al.) | 24/122 (19.6%) | 19/33 (57.5%) |

TABLE 17
Accuracy of predictions in cell lines and patients

| | Drugs | | | | | | |
|---|---|---|---|---|---|---|---|
| Validations | Topotecan | Adriamycin | Etoposide | 5-Flourouracil | Paclitaxed | Cytoxan | Doceaxel |
| In Vitro Data | | | | | | | |
| Accuracy | 18/20 (90%) | 18/25 (86%) | 21/24 | 21/24 | 26/28 (92.8%) | 25/29 | P < 0.001** |
| | | | (87%) | (87%) | | (86.2%) | |
| PPV | 12/14 (86%) | 13/13 (100%) | 6/8 | 14/14 | 21/21 (100%) | 13/15 | |
| | | | (75%) | (100%) | | (86.6%) | |
| NPV | 6/6 (100%) | 5/8 (62.5%) | 15/16 | 7/10 | 5/7 (71.5%) | 12/14 | |
| | | | (94%) | (70%) | | (86%) | |
| In Vivo (Patient) | | | | | | | |
| Data | | | | | | | Breast |
| Accuracy | 40/48 (83.2%) | 99/122 (81%) | | | 28/35 (80%) | | 22/24 |
| | | | | | | | (91.6%) |
| PPV | 17/22 (77.34%) | 19/33 (57.6%) | | | 20/28 (71.4%) | | 11/13 |
| | | | | | | | (85.7%) |
| NPV | 23/26 (88.5%) | 80/89 (89.8%) | | | 7/7 (100%) | | 11/11 |
| | | | | | | | (100%) |
| | | | | | | | Ovarian |
| | | | | | | | 12/14 |
| | | | | | | | (85.7%) |
| | | | | | | | 6/7 |
| | | | | | | | (85.7%) |
| | | | | | | | 6/7 |
| | | | | | | | (85.7%) |

PPV—positive predictive value,
NPV—negative predictive value.
**Determining accuracy for the docetaxel predictor in the IJC cellline data set was not possible since docetaxel was not one of the drugs studied. Instead, the docetaxel predictor was validated in two independent cell line experiments, correlating predicted probability of response to docetaxel in vitro with actual IC50 of docetaxel by cell line (FIG. 1C).

TABLE 18
Comparison of different predictors

| | | | | Predictor of |
|---|---|---|---|---|
| | | | Genomic predictor of | response to |
| | Docetaxel | Docetaxel | response to TFAC | TFAC |
| Validations/ | predictor | predictor (Chang | chemotherapy (Potti | chemotherapy |
| Predictors | (Potti et al.) | et al.) | et al.) | (Pusztai et al.) |
| Breast | | | | |
| neoadjuvant data | | | | |
| (Chang et al.) | | | | |
| Accuracy | 22/24 | 87.5% | | |
| | (91.6%) | | | |
| PPV | 13/13 | 92% | | |
| | (85.7%) | | | |
| NPV of | 11/11 | 83% | | |
| ROC | (100%) | | | |
| | 0.97 | 0.96 | | |
| MDACC data | | | | |
| (Pusztai et al.) | | | | |
| Accuracy | | | 42/51 (82.3%) | 74% |
| PPV | | | 11/18 (61.1%) | 44% |
| NPV | | | 31/33 (94%) | 93% |

PPV—positive predictive value,
NPV—negative predictive value.
**For both the Chang and Pusztai data, the actual numbers of predicted responders was not available, just the predictive accuracies. Also, the predictive accuracy reported for the Chang data is not in an independent validation, instead it is for leave-one cross out validation.

What is claimed is:

1. A method for predicting responsiveness of a cancer to a chemotherapeutic agent comprising:
a) comparing a first gene expression profile of the cancer to a chemotherapy responsivity predictor set of gene expression profiles, the first gene expression profile and the chemotherapy responsivity predictor set each comprising at least five genes from one of Tables 1-8, wherein Tables 1-8 comprise the chemotherapy responsivity predictor set for 5-fluorouracil, adriamycin, cytotoxan, docetaxol, etoposide, taxol, topotecan and PI3 kinase inhibitors, respectively; and
b) using the comparison of step (a) to predict the responsiveness of the cancer to the chemotherapeutic agent.

2. The method of claim 1, wherein the chemotherapeutic agent is an inhibitor of the PI3kinase pathway and the first gene expression profile and the chemotherapy responsivity predictor set each comprise at least five genes from Table 4.

3. The method of claim 1, wherein the chemotherapeutic agent is an inhibitor of the Src pathway and the first gene expression profile and the chemotherapy responsivity predictor set each comprise at least five genes from Table 7.

4. The method of claim 1, wherein the first gene expression profile is obtained by analyzing a nucleic acid sample from the cancer.

5. The method of claim 1, wherein the first gene expression profile is obtained by analyzing a sample from a tumor or ascites.

6. The method of claim 1, wherein the first gene expression profile is determined using a nucleic acid microarray.

7. The method of claim 1, wherein the first gene expression profile and the chemotherapy responsivity predictor set each comprises at least 10 genes.

8. The method of claim 1, wherein the first gene expression profile and the chemotherapy responsivity predictor set each comprises at least 20 genes.

9. The method of claim 1, wherein the cancer is from an individual and wherein step (b) identifies the individual as a complete responder or as an incomplete responder to the chemotherapeutic agent.

10. The method of claim 1, wherein the first gene expression profile is compared to at least two chemotherapy responsivity predictor sets each comprising at least five genes from the corresponding Tables 1-8.

11. The method of claim 1, wherein the cancer is selected from the group consisting of lung, breast, ovarian, prostrate, renal, colon, leukemia, skin, and brain cancer.

12. The method of claim 1, wherein the chemotherapy responsivity predictor set is defined by extracting a single dominant value using singular value decomposition (SVD) and determining the value of the chemotherapy responsivity predictor set in the cancer.

13. The method of claim 1, wherein step (b) comprises applying one or more statistical models to the comparison of step (a), each model producing a statistical probability of the sensitivity of the cancer to the chemotherapeutic agent.

14. The method of claim 13, wherein the statistical model is a binary regression model.

15. The method of claim 13, wherein the statistical model is a tree model, the tree model including one or more nodes, each node representing a metagene, each node including a statistical probability of sensitivity of the cancer to the chemotherapeutic agent.

16. The method of claim 1, wherein the method predicts responsiveness to the chemotherapeutic agent with at least 80% accuracy.

17. The method of claim 1, wherein the chemotherapy responsivity predictor set is developed using at least one resistant cell line and at least one sensitive cell line of one of Tables 9-15, Tables 9-15 listing cell lines sensitive or resistant to 5-fluorouracil, adriamycin, cytotoxan, docetaxol, etoposide, taxol, and topotecan, respectively.

18. A method of developing a treatment plan for an individual with cancer comprising using the predicted responsivity of a cancer to a chemotherapeutic agent obtained by the method of claim 1 to develop a treatment plan.

19. The method of claim 18, wherein the treatment plan includes administering an effective amount of a chemotherapeutic agent to the individual with the cancer if the cancer is predicted to respond to the chemotherapeutic agent.

20. The method of claim 18, further comprising comparing the first gene expression profile to an alternative chemotherapy responsivity predictor set of gene expression profiles predictive of responsivity to alternative chemotherapeutic agents; predicting responsiveness of the cancer to the alternative chemotherapeutic agents and administering an alternative chemotherapeutic agent to the individual with the cancer.

21. The method of claim 20, wherein the alternative chemotherapeutic agent is selected from the group comprising docetaxel, paclitaxel, abraxane, topotecan, adriamycin, etoposide, fluorouracil (5-FU), cyclophosphamide, denopterin, edatrexate, methotrexate, nolatrexed, pemetrexed, piritrexim, pteropterin, raltitrexed, trimetrexate, cladribine, ctofarabine, fludarabine, 6-mercaptopurine, nelarabine, thiamiprine, thioguanine, tiazofurin, ancitabine, azacibdine, 6-azauridine, capecitabine, carmofur, cytarabine, decitabine, doxifluridine, enocitabine, floxuridine, fluorouracil, gemcitabine, tegafur, troxacitabine, pentostatin, hydroxyurea, cytosine arabinoside.

22. The method of claim 18, wherein the plan includes administering the chemotherapeutic agent before, after or concurrently with the administration of one or more alternative chemotherapeutic agents.

23. The method of claim 18, wherein the alternative chemotherapeutic agent targets a signal transduction pathway.

24. The method of claim 23, wherein the first gene expression profile of the cancer comprises at least one gene expression profile indicative of deregulation of the signal transduction pathway.

25. The method of claim 23, wherein the alternative chemotherapeutic agent is selected from inhibitors of a signal transduction pathway selected from the group consisting of Src, E2F3, Myc, PI3kinase and β-catenin.

26. The method of claim 18, wherein the cancer is predicted to be responsive to more than one chemotherapeutic agent.

27. The method of claim 26, wherein the treatment plan administering an effective amount of at least two chemotherapeutic agents to the individual with the cancer.

28. The method of claim 27, wherein the plan includes administering at least two chemotherapeutic agents before, after or concurrently with each other.

29. The method of claim 18, wherein the treatment plan has an estimated efficacy of at least 50%.

30. A kit comprising a gene chip for predicting responsivity of a cancer to a chemotherapeutic agent comprising nucleic acids capable of detecting at least five genes selected from Tables 1-8 and instructions for predicting responsivity of a cancer to the chemotherapeutic agent.

31. A computer readable medium comprising gene expression profiles and corresponding responsivity information for chemotherapeutic agents comprising at least five genes from any of Tables 1-8.
